(19) United States
(12) Patent Application Publication
(10) Pub. No.: US 2019/0031676 A1
(43) Pub. Date: Jan. 31, 2019

(54) ANTIBACTERIAL ANNULATED PYRROLIDIN-2-ONE DERIVATIVES

(71) Applicant: IDORSIA PHARMACEUTICALS LTD, Allschwil (CH)

(72) Inventors: Philippe PANCHAUD, Allschwil (CH); Christine SCHMITT, Allschwil (CH); Jean-Philippe SURIVET, Allschwil (CH)

(73) Assignee: IDORSIA PHARMACEUTICALS LTD, Allschwil (CH)

(21) Appl. No.: 15/755,893

(22) PCT Filed: Aug. 26, 2016
(86) PCT No.: PCT/EP2016/070203

§ 371 Date: Feb. 27, 2018

(30) Foreign Application Priority Data
Aug. 28, 2015 (EP) PCT/EP2015/069757

(51) Int. Cl. C07D 495/04; C07D 209/46; A61P 31/04

(52) U.S. Cl. CPC C07D 495/04; A61P 31/04; C07D 209/46

(57) ABSTRACT

The invention relates to antibacterial compounds of formula I

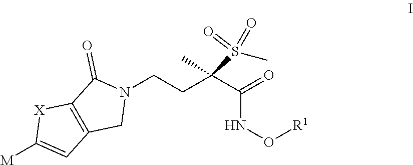

wherein
X represents sulphur or CH═CH;
R¹ represents H, PO₃H₂, SO₃H, phosphonooxymethyl or the group —CO—R² wherein R² is as defined in the claims
M is one of the groups M^{A} and M^{B} represented below

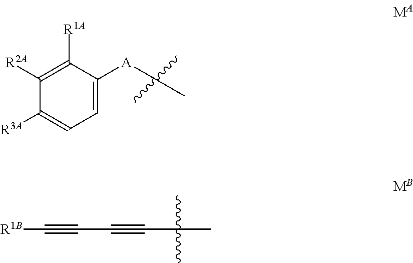

wherein A represents a bond or C≡C and R^{1A}, R^{2A}, R^{3A} and R^{1B} are as defined in the claims;
   and to salts thereof.

The present invention concerns antibacterial annulated pyrrolidin-2-one derivatives, pharmaceutical compositions containing them and uses of these compounds in the manufacture of medicaments for the treatment of bacterial infections. These compounds are useful antimicrobial agents effective against a variety of human and veterinary pathogens, especially Gram-negative aerobic and anaerobic bacteria. The compounds of the present invention can optionally be employed in combination, either sequentially or simultaneously, with one or more therapeutic agents effective against bacterial infections.

The intensive use of antibiotics has exerted a selective evolutionary pressure on microorganisms to produce genetically based resistance mechanisms. Modern medicine and socio-economic behaviour exacerbate the problem of resistance development by creating slow growth situations for pathogenic microbes, e.g. in artificial joints, and by supporting long-term host reservoirs, e.g. in immune-compromised patients.

In hospital settings, an increasing number of strains of Staphylococcus aureus, Streptococcus pneumoniae, Enterococcus spp., Enterobacteriaceae such as Klebsiella pneumoniae, Acinetobacter baumannii and Pseudomonas aeruginosa, major sources of infections, are becoming multi-drug resistant and therefore difficult if not impossible to treat. This is particularly the case for Gram-negative organisms where the situation is getting worrisome since no novel agents have been approved for decades and the development pipeline looks empty.

Therefore, there is an important medical need for new antibacterial compounds addressing Gram-negative resistant bacteria, in particular third generation cephalosporins- and carbapenem-resistant Klebsiella pneumoniae and multi-drug-resistant Pseudomonas aeruginosa and Acinetobacter baumannii. One way to tackle the problem of cross resistance to established classes of antibiotics is to inhibit a new target. In this respect, LpxC, which is an essential enzyme in the biosynthesis of lipopolysaccharides (a major constituent of the outer membrane of Gram-negative bacteria), has received some attention and several patent applications relating to LpxC inhibitors have been published recently.

For example, WO 2011/045703, WO 2011/073845, WO 2012/120397, WO 2012/137094, WO 2012/137099, WO 2013/170165 and WO 2015/066413 describe antibacterial compounds having a N-hydroxy-2-methyl-2-(methylsulfonyl)butanamide side chain bound to a monocyclic aromatic or heteroaromatic ring system.

Furthermore WO 2013/170165 describes notably antibacterial compounds of formula (A0)

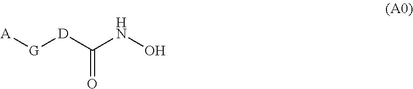

wherein A is a substituted alkyl group, wherein at least one substituent is hydroxy, or A is a substituted cycloalkyl group, wherein at least one substituent is hydroxy or hydroxyalkyl; G is a group comprising at least one carbon-carbon double or triple bond and/or a phenyl ring; and D represents a group selected from

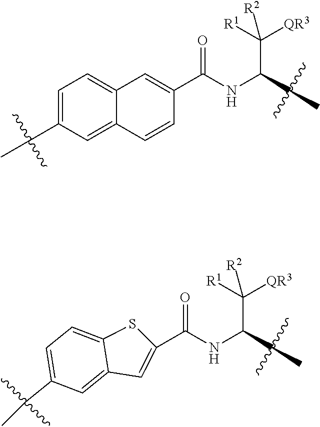

wherein Q is O or NR, wherein R is H or an unsubstituted (C₁-C₃)alkyl; R¹ and R² independently are selected from the group consisting of H and substituted or unsubstituted (C₁-C₃)alkyl, or R¹ and R², together with the carbon atom to which they are attached, form an unsubstituted (C₃-C₄)cycloalkyl group or an unsubstituted 4-6 membered heterocyclic group; and R³ is selected from the group consisting of hydrogen, substituted or unsubstituted (C₁-C₃)alkyl, substituted or unsubstituted cycloalkyl, substituted or unsubstituted cycloalkylalkyl, substituted or unsubstituted aryl, substituted or unsubstituted arylalkyl, substituted or unsubstituted heterocyclyl, substituted or unsubstituted heterocyclylalkyl, substituted or unsubstituted heteroaryl, and substituted or unsubstituted heteroarylalkyl.

In WO 2015/036964, we have reported antibacterial 2H-indazole derivatives of general formula (A1)

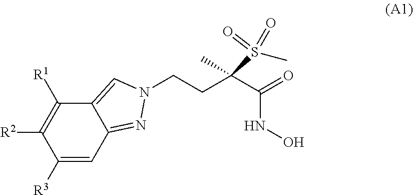

wherein

R¹ is H or halogen; R² is (C₃-C₄)alkynyloxy or the group M; R³ is H or halogen; M is one of the groups M^{A} and M^{B} represented below

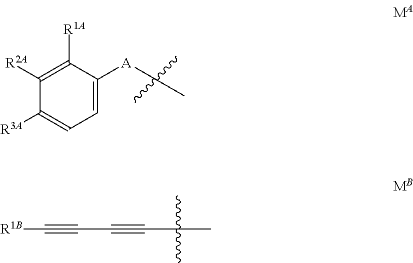

wherein A is a bond, CH₂CH₂, CH═CH or C≡C; R^{1A} represents H or halogen; R^{2A} represents H, alkoxy or halogen; R^{3A} represents H, alkoxy, hydroxyalkoxy, thioalkoxy, trifluoromethoxy, amino, dialkylamino, hydroxyalkyl, 1-hydroxymethyl-cycloprop-1-yl, trans-2-hydroxymethyl-cycloprop-1-yl, 1,2-dihydroxyethyl, 3-hydroxyoxetan-3-yl, 3-(hydroxyalkyl)oxetan-3-yl, 3-aminooxetan-3-yl, 3-(dialkylamino)oxetan-3-yl, 3-hydroxythietan-3-yl, morpholin-4-ylalkoxy, morpholin-4-ylalkyl, oxazol-2-yl or [1,2,3]triazol-2-yl; and R^{1B} represents 3-hydroxyoxetan-3-yl, 3-hydroxythietan-3-yl, hydroxyalkyl, aminoalkyl, trans-2-hydroxymethyl-cycloprop-1-yl or 4-hydroxytetrahydro-2H-pyran-4-yl.

In WO 2015/091741, we have reported antibacterial 1H-indazole derivatives of general formula (A2)

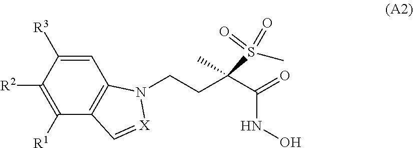

wherein

X represents N or CH;

R¹ represents H or halogen;

R² represents (C₃-C₄)alkynyloxy or the group M;

R³ represents H or halogen;

M is one of the groups M^{A} and M^{B} represented below

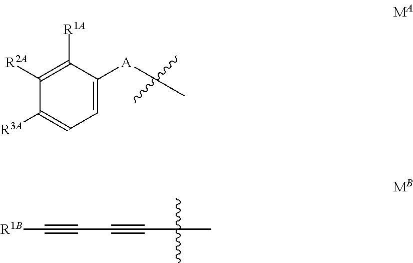

wherein A represents a bond, CH₂CH₂, CH═CH or C≡C;

R^{1A} represents H or halogen;

R^{2A} represents H, (C₁-C₃)alkoxy or halogen;

R^{3A} represents H, (C₁-C₃)alkoxy, hydroxy(C₁-C₄)alkoxy, (C₁-C₃)thioalkoxy, trifluoromethoxy, amino, hydroxy(C₁-C₄)alkyl, 2-hydroxyacetamido, 1-hydroxymethyl-cycloprop-1-yl, trans-2-hydroxymethyl-cycloprop-1-yl, 1,2-dihydroxyethyl, 3-hydroxyoxetan-3-yl, 3-(hydroxy(C₁-C₃)alkyl)oxetan-3-yl, 3-aminooxetan-3-yl, 3-hydroxythietan-3-yl, morpholin-4-yl(C₂-C₃)alkoxy, morpholin-4-yl-(C₁-C₂)alkyl, oxazol-2-yl or [1,2,3]triazol-2-yl; and

R^{1B} represents 3-hydroxyoxetan-3-yl, 3-hydroxythietan-3-yl, hydroxy(C₁-C₃)alkyl, amino(C₁-C₃)alkyl, 1-hydroxymethyl-cycloprop-1-yl or trans-2-hydroxymethyl-cycloprop-1-yl.

In WO 2015/132228, we have reported antibacterial 1,2-dihydro-3H-pyrrolo[1,2-c]imidazol-3-one derivatives of general formula (A3)

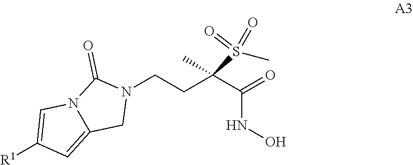

wherein R¹ is the group M; M is one of the groups M^{A} and M^{B} represented below

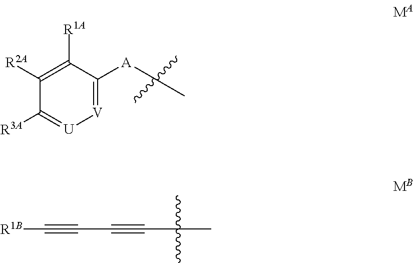

wherein A is a bond, CH═CH or C≡C; U is N or CH; V is N or CH; R^{1A} is H or halogen; R^{2A} is H, (C₁-C₃)alkoxy or halogen; R^{3A} is H, (C₁-C₃)alkoxy, hydroxy(C₂-C₄)alkoxy, (C₁-C₃)alkoxy(C₁-C₃)alkoxy, (C₁-C₃)thioalkoxy, trifluoromethoxy, amino, hydroxy(C₁-C₄)alkyl, (C₁-C₃)alkoxy(C₁-C₄)alkyl, 3-hydroxy-3-methylbut-1-yn-1-yl, 2-hydroxyacetamido, (carbamoyloxy)methyl, 1-hydroxymethyl-cycloprop-1-yl, 1-aminomethyl-cycloprop-1-yl, 1-(carbamoyloxy)methyl-cycloprop-1-yl, 1-(morpholin-4-yl)methylcycloprop-1-yl, trans-2-hydroxymethyl-cycloprop-1-yl, 1,2-dihydroxyethyl, 3-hydroxyoxetan-3-yl, 3-(hydroxy(C₁-C₃)alkyl)oxetan-3-yl, 3-aminooxetan-3-yl, 3-hydroxythietan-3-yl, morpholin-4-yl(C₂-C₃)alkoxy, [4-N—(C₁-C₃)alkylpiperazin-1-yl](C₁-C₃)alkyl, morpholin-4-yl-(C₁-C₂)alkyl, [1,2,3]triazol-2-yl or 3-[hydroxy(C₂-C₃)alkyl]-2-oxo-imidazolidin-1-yl; and R^{1B} is 3-hydroxyoxetan-3-yl, 3-hydroxythietan-3-yl, 3-(hydroxy(C₁-C₃)alkyl)oxetan-3-yl, hydroxy(C₁-C₃)alkyl, 1,2-dihydroxyethyl, amino(C₁-C₃)alkyl, 1-hydroxymethyl-cycloprop-1-yl, trans-2-hydroxymethyl-cycloprop-1-yl, trans-(cis-3,4-dihydroxy)-cyclopent-1-yl or 3-hydroxymethylbicyclo[1,1,1]pentan-1-yl.

In WO 2015/173329, we have reported antibacterial quinazoline-4(3H)-one derivatives of general formula (A4)

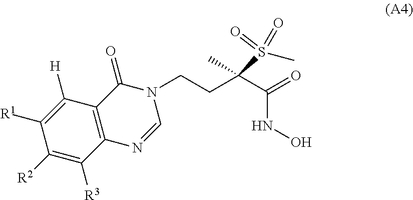

wherein R¹ is H or halogen; R² is the group M; R³ is H or halogen; M is one of the groups M^{A} and M^{B} represented below

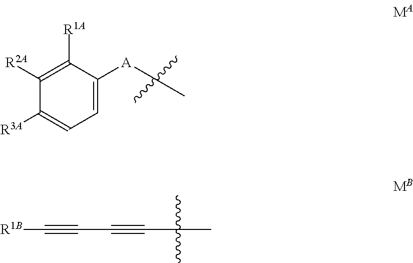

wherein A represents a bond or C≡C; R^{1A} is H or halogen; R^{2A} is H, (C₁-C₃)alkoxy or halogen; R^{3A} is H, (C₁-C₃)alkoxy, hydroxy(C₂-C₄)alkoxy, hydroxy(C₁-C₄)alkyl, 1,2-dihydroxyethyl, di(C₁-C₃)alkylamino, 1-hydroxymethyl-cycloprop-1-yl, 1-((dimethylglycyl)oxy)methyl-cycloprop-1-yl, 3-hydroxyoxetan-3-yl, morpholin-4-yl-(C₁-C₂)alkyl or morpholin-4-yl(C₂-C₃)alkoxy; and R^{1B} is hydroxy(C₁-C₃)alkyl, amino(C₁-C₃)alkyl, 1,2-dihydroxyprop-3-yl, 1-amino-cycloprop-1-yl, 1-hydroxymethyl-cycloprop-1-yl, trans-2-hydroxymethyl-cycloprop-1-yl, trans-2-aminomethyl-cycloprop-1-yl, trans-2-hydroxymethyl-1-methyl-cycloprop-1-yl, trans-2-hydroxymethyl-2-methyl-cycloprop-1-yl, 1-(1,2-dihydroxyethyl)-cycloprop-1-yl, trans-2-(1,2-dihydroxyethyl)-cycloprop-1-yl, 3-hydroxyoxetan-3-yl, 3-(hydroxy(C₁-C₃)alkyl)oxetan-3-yl, 3-hydroxythietan-3-yl, trans-(cis-3,4-dihydroxy)-cyclopent-1-yl, 3-(2-aminoacetamido)cyclopentyl or 3-hydroxymethylbicyclo[1,1,1]pentan-1-yl.

In WO 2016/079688, we have reported antibacterial benzothiazole derivatives of general formula (A5)

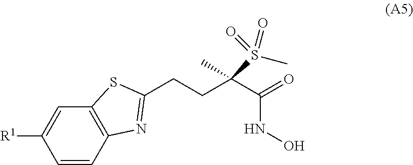

wherein

R¹ is the group M, whereby M is one of the groups M^{A} and M^{B} represented below

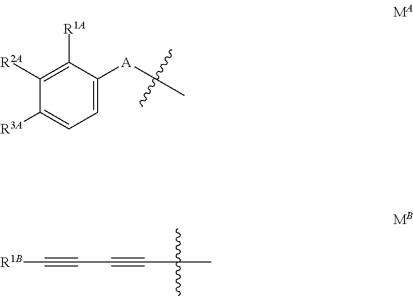

wherein A represents a bond or C≡C;

R^{1A} is H or halogen;

R^{2A} is H or halogen; and

R^{3A} is H, (C₁-C₃)alkoxy, hydroxy(C₂-C₄)alkoxy, hydroxy(C₁-C₄)alkyl, dihydroxy(C₂-C₄)alkyl, 2-hydroxyacetamido, 1-hydroxymethyl-cycloprop-1-yl, trans-2-hydroxymethyl-cycloprop-1-yl, 3-hydroxyoxetan-3-yl, 3-(hydroxy(C₁-C₃)alkyl)oxetan-3-yl, 3-aminooxetan-3-yl or 1-aminocycloprop-1-yl; and wherein R^{1B} is hydroxy(C₁-C₄)alkyl, dihydroxy(C₂-C₄)alkyl, amino(C₁-C₄)alkyl, di(C₁-C₄)alkylamino(C₁-C₃)alkyl, 1-amino-cycloprop-1-yl, 1-hydroxymethyl-cycloprop-1-yl, trans-2-hydroxymethyl-cycloprop-1-yl, trans-2-aminomethyl-cycloprop-1-yl, trans-2-hydroxymethyl-1-methyl-cycloprop-1l-yl, trans-2-hydroxymethyl-2-methyl-cycloprop-1-yl, cis-1-fluoro-2-(hydroxymethyl)cycloprop-1-yl, cis-2-fluoro-2-(hydroxymethyl)cycloprop-1-yl, 2-(1,2-dihydroxyethyl)-cycloprop-1-yl, 1-(hydroxymethyl)-cyclobutan-1-yl, cis-3-(hydroxymethyl)-1-hydroxy-cyclobutan-1-yl, 3-hydroxyoxetan-3-yl, 3-hydroxyoxetan-3-yl-(C₁-C₃)alkyl, 3-aminooxetan-3-yl, 3-hydroxymethyl-oxetan-3-yl, trans-(cis-3,4-dihydroxy)-cyclopent-1-yl, 3-hydroxymethylbicyclo[1,1,1]pentan-1-yl, 4-hydroxytetrahydro-2H-pyran-4-yl, (3R,6S)-3-aminotetrahydro-2H-pyran-6-yl, piperidin-4-yl, 1-(2-hydroxyacetyl)piperidin-4-yl, 3-hydroxythietan-3-yl, 1-(2-hydroxyacetyl)azetidin-3-yl or 1-glycylazetidin-3-yl;

and salts thereof.

Besides, in Montgomery et al., J. Med. Chem. (2012), 55(4), 1662-1670, yet further LpxC inhibitors are disclosed, among others the compound of formula (A6)

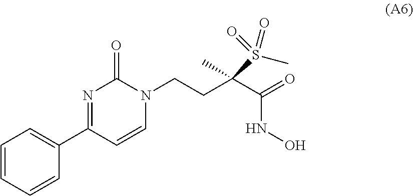 [SMILES: C[C@@](CCn1ccc(-c2ccccc2)nc1=O)(C(=O)NO)S(C)(=O)=O]

The instant invention provides new antibacterial annulated pyrrolidin-2-one derivatives, namely the compounds of formula I described herein.

Various embodiments of the invention are presented hereafter:

1) The present invention relates to compounds of formula I

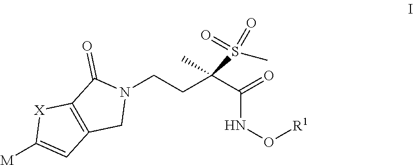

wherein

X represents sulphur or CH═CH;

R¹ represents H, PO₃H₂, SO₃H, phosphonooxymethyl or the group L represented below

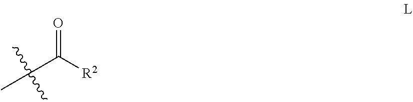

wherein

R² represents (C₁-C₄)alkylamino(C₁-C₄)alkyl, di(C₁-C₄)alkylamino(C₁-C₄)alkyl, phosphonooxy(C₁-C₄)alkyl, phosphonooxymethoxy, 2-(phosphonooxy-(C₁-C₄)alkyl)-phenyl, [2-(phosphonooxy-(C₁-C₄)alkyl)-phenyl]-(C₁-C₄)alkyl, or (2-(phosphonooxy)-phenyl)-(C₁-C₄)alkyl (especially 2-(2-(phosphonooxy)-phenyl)-ethyl);

M is one of the groups M^{A} and M^{B} represented below

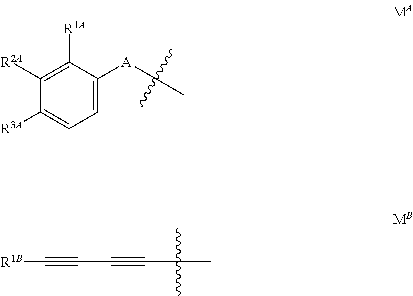

wherein A represents a bond or C≡C;

R^{1A} represents H or halogen (if halogen, then especially fluorine);

R^{2A} represents H or halogen;

R^{3A} represents H, (C₁-C₃)alkoxy (especially methoxy), hydroxy(C₂-C₄)alkoxy, hydroxy(C₁-C₄)alkyl (especially 1-hydroxy-2-methylpropan-2-yl), 1,2-dihydroxyethyl, (3-fluoroazetidin-1-yl)methyl, 3-fluoro-1-(oxetan-3-yl)azetidin-3-yl, 3-fluoro-1-methyl-azetidin-3-yl, (4-hydroxy-3-fluoropiperidin-1-yl)methyl, (4-hydroxy-3,3-difluoropiperidin-1-yl)methyl, (3-hydroxyazetidin-1-yl)methyl, 3-(w-hydroxy(C₂-C₄)alkyl)-azetidin-1-yl, 1-(oxetan-3-yl)azetidin-3-yl, 1-(oxetan-3-ylmethyl)azetidin-3-yl or (4-hydroxypiperidin-1-yl)methyl;

R^{1B} represents amino(C₁-C₃)alkyl, 1-amino-cycloprop-1-yl, trans-2-(2-dimethylaminoacetoxymethyl)-cycloprop-1-yl, 1-(2-dimethylaminoacetoxymethyl)-cycloprop-1-yl, 1-(3-hydroxyazetidine)-1-carbonyloxymethyl, 1-hydroxymethyl-cycloprop-1-yl, trans-2-hydroxymethyl-cycloprop-1-yl, 1-fluoro-2-hydroxymethyl-cycloprop-1-yl, 2-fluoro-2-hydroxymethyl-cycloprop-1-yl, 1-{[(2-(phosphonooxy-(C₁-C₄)alkyl)-phenyl)-(C₁-C₄)alkyl]carbonyloxymethyl}cycloprop-1-yl, 1-{[2-(phosphonooxy-(C₁-C₄)alkyl)-phenyl]carbonyloxymethyl}cycloprop-1-yl, 1-{[(2-phosphonooxy-phenyl)-(C₁-C₄)alkyl]carbonyloxymethyl}cycloprop-1-yl, trans-2-{[(2-(phosphonooxy-(C₁-C₄)alkyl)-phenyl)-(C₁-C₄)alkyl]carbonyloxymethyl}cycloprop-1-yl, trans-2-{[2-(phosphonooxy-(C₁-C₄)alkyl)-phenyl]carbonyloxymethyl}cycloprop-1-yl, trans-2-{[(2-phosphonooxy-phenyl)-(C₁-C₄)alkyl]carbonyloxymethyl}cycloprop-1-yl, 2-fluoro-2-{[(2-(phosphonooxy-(C₁-C₄)alkyl)-phenyl)-(C₁-C₄)alkyl]carbonyloxymethyl}cycloprop-1-yl, 2-fluoro-2-{[2-(phosphonooxy-(C₁-C₄)alkyl)-phenyl]carbonyloxymethyl}cycloprop-1-yl, 2-fluoro-2-{[(2-phosphonooxy-phenyl)-(C₁-C₄)alkyl]carbonyloxymethyl}cycloprop-1-yl, 2-fluoro-2-(phosphonooxymethyl)-cycloprop-1-yl, 1-methyl-2-hydroxymethyl-cycloprop-1-yl, 2-hydroxymethyl-2-methylcycloprop-1-yl, 1-(2-hydroxyacetyl)-azetidin-3-yl, trans-(cis-3,4-dihydroxy)-cyclopent-1-yl, 2-(1,2-dihydroxyethyl)cycloprop-1-yl, 1-(dimethylamino)cycloprop-1-yl, 2-(morpholinomethyl)cycloprop-1-yl, 2-((3-fluoroazetidin-1-yl)methyl)cyclopropyl, N—(C₁-C₄)alkyl-azetidin-3-yl (especially N-methylazetidin-3-yl), N—(C₃-C₆)cycloalkyl-azetidin-3-yl, 3-fluoro-1-methyl-azetidin-3-yl, 1-(methylamino)cycloprop-1-yl, N-(ω-hydroxy(C₂-C₄)alkyl)-azetidin-3-yl (especially N-(2-hydroxyethyl)azetidin-3-yl), 2-(hydroxymethyl)-1-methylazetidin-3-yl, N-(2-hydroxy-2-methylpropyl)azetidin-3-yl, N-(ω-halo(C₂-C₄)alkyl)-azetidin-3-yl, 1-(3-hydroxypropyloxycarbonyl)-azetidin-3-yl, 2-(fluoromethyl)-1-methylazetidin-3-yl, N-(3-hydroxycyclobutyl)azetidin-3-yl, N-(oxetan-3-ylmethyl)azetidin-3-yl, N-(3-hydroxyoxetan-3-ylmethyl)azetidin-3-yl, N-(tetrahydrofuran-3-yl)azetidin-3-yl, N-(tetrahydro-2H-pyran-4-yl)azetidin-3-yl, trans-2-hydroxymethyl-1-methylazetidin-4-yl, (5-hydroxymethyl)-1-methylpyrrolidin-3-yl, 4-fluoro-1-methylpiperidin-4-yl, or 1-(oxetan-3-yl)-azetidin-3-yl;

and to salts (in particular pharmaceutically acceptable salts) of such compounds of formula I.

It is understood that groups —O—R¹ in the fragment —CO—NH—O—R¹ wherein R¹ is not H represent prodrugs of the —CO—NH—OH group. It is further understood that the R_{1B} subgroups 2-dimethylaminoacetoxy, phosphonooxy, [(2-(phosphonooxy-(C₁-C₄)alkyl)-phenyl)-(C₁-C₄)alkyl]carbonyloxy, [2-(phosphonooxy-(C₁-C₄)alkyl)-phenyl]carbonyloxy, and [(2-phosphonooxy-phenyl)-(C₁-C₄)alkyl]carbonyloxy represent prodrugs of the corresponding hydroxy group.

In particular,
the prodrug group [2-(phosphonooxy-(C₁-C₄)alkyl)-phenyl]carbonyloxy notably refers to:

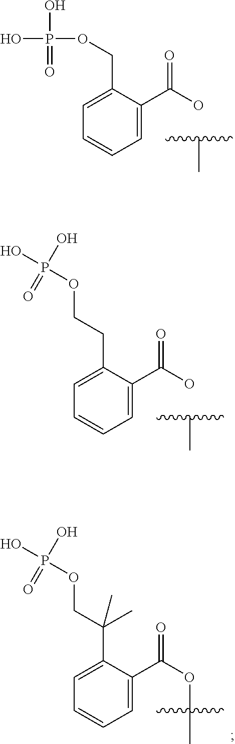

the prodrug group [(2-(phosphonooxy-(C₁-C₄)alkyl)-phenyl)-(C₁-C₄)alkyl]carbonyloxy notably refers to:

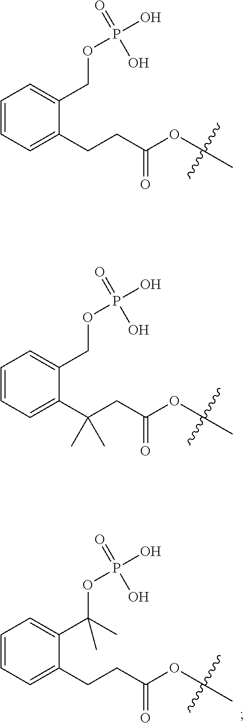 [SMILES: *OC(=O)CCc1ccccc1COP(=O)(O)O ; *OC(=O)CC(C)(C)c1ccccc1COP(=O)(O)O ; *OC(=O)CCc1ccccc1C(C)(C)OP(=O)(O)O]

the prodrug group [(2-phosphonooxy-phenyl)-(C₁-C₄)alkyl]carbonyloxy notably refers to:

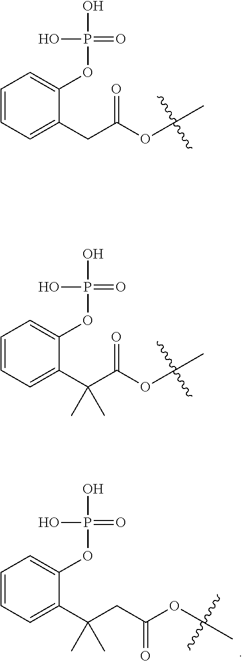 [SMILES: *OC(=O)Cc1ccccc1OP(=O)(O)O ; *OC(=O)C(C)(C)c1ccccc1OP(=O)(O)O ; *OC(=O)CC(C)(C)c1ccccc1OP(=O)(O)O]

The following paragraphs provide definitions of the various chemical moieties for the compounds according to the invention and are intended to apply uniformly throughout the specification and claims, unless an otherwise expressly set out definition provides a broader or narrower definition:
The term “alkyl” or “alkyl group”, used alone or in combination, refers to a straight or branched saturated hydrocarbon group containing from one to four carbon atoms. The term “(Cₓ-C_{y})alkyl” (x and y each being an integer) refers to a straight or branched saturated hydrocarbon group containing x to y carbon atoms. For example, a (C₁-C₄)alkyl group contains from one to four carbon atoms.
The term “cycloalkyl”, used alone or in combination, refers to a saturated cyclic hydrocarbon group containing from three to six carbon atoms. The term “(Cₓ-C_{y})cycloalkyl” (x and y each being an integer) refers to a cycloalkyl group as defined containing x to y carbon atoms. A (C₃-C₆)cycloalkyl group thus includes cyclopropyl, cyclobutyl, cyclopentyl and cyclohexyl.
The term “hydroxyalkyl”, used alone or in combination, refers to an alkyl group as defined before wherein one hydrogen atom has been replaced by a hydroxy group. The term “hydroxy(Cₓ-C_{y})alkyl” (x and y each being an integer) refers to a hydroxyalkyl group as defined which contains x to y carbon atoms. For example, a hydroxy(C₁-C₄)alkyl group is a hydroxyalkyl group as defined before which contains from one to four carbon atoms. The term “w-hydroxy(C₂-C₄)alkyl”, used alone or in combination, refers to a straight saturated hydrocarbon group containing from two to four carbon atoms, i.e. ethyl, n-propyl and n-butyl, wherein one hydrogen atom of the terminal carbon atom has been replaced by a hydroxy group. A w-hydroxy(C₂-C₄)alkyl group thus includes 2-hydroxy-ethyl, 3-hydroxy-propyl and 4-hydroxy-butyl.
The term “haloalkyl”, used alone or in combination, refers to an alkyl group as defined before containing one to four carbon atoms in which one or more (and possibly all) hydrogen atoms have been replaced by halogen atoms. The term “(Cₓ-C_{y})haloalkyl” (x and y each being an integer) refers to a haloalkyl group as defined before containing x to y carbon atoms. In a sub-embodiment, an “ω-(C₂-C₄)haloalkyl” group, used alone or in combination, refers to an alkyl group of two or four carbon atoms in which one, two or three terminal hydrogen atoms have been replaced by halogen atoms. Representative examples of ω-(C₂-C₄)haloalkyl groups include especially the ω-(C₂)fluoroalkyl groups 2-fluoroethyl, 2,2-difluoroethyl and 2,2,2-trifluoroethyl. Preferred ω-(C₂)fluoroalkyl groups as used in the definitions of substituent R^{1B} are 2-fluoroethyl and 2,2,2-trifluoroethyl, especially 2-fluoroethyl.
The term “aminoalkyl”, used alone or in combination, refers to an alkyl group as defined before wherein one hydrogen atom has been replaced by an amino group. The term “amino(Cₓ-C_{y})alkyl” (x and y each being an integer) refers to an aminoalkyl group as defined which contains x to y carbon atoms. For example, an amino(C₁-C₃)alkyl group is an aminoalkyl group as defined before which contains from one to three carbon atoms.
The term “alkylaminoalkyl”, used alone or in combination, refers to an aminoalkyl group as defined before wherein one hydrogen atom of the amino group has been replaced by an alkyl group wherein the alkyl group is as defined before. The term “(C_{x′}-C_{y′})alkylamino(Cₓ-C_{y})alkyl” (x′, x, y′ and y each being an integer) refers to an alkylaminoalkyl group as defined which contains x′ to y′ and x to y carbon atoms. For example, a (C₁-C₄)alkylamino(C₁-C₄)alkyl group is an alkylaminoalkyl group as defined before wherein the two alkyl groups independent of each other contain from one to four carbon atoms.
The term “dialkylaminoalkyl”, used alone or in combination, refers to an aminoalkyl group as defined before wherein the two hydrogen atoms of the amino group have been independently replaced by an alkyl group wherein the alkyl group is as defined before. The term “di(C_{x′}-C_{y′})alkylamino(Cₓ-C_{y})alkyl” (x′, x, y′ and y each being an integer) refers to a dialkylaminoalkyl group as defined which contains x′ to y′ and x to y carbon atoms wherein the two (C_{x′}-C_{y′})alkyl groups may be the same or different. For example, a di(C₁-C₄)alkylamino(C₁-C₄)alkyl group is a dialkylaminoalkyl group as defined before wherein the three alkyl groups independent of each other contain from one to four carbon atoms.
The term “alkoxy”, used alone or in combination, refers to a straight or branched chain alkoxy group containing from one to four carbon atoms. The term “(Cₓ-C_{y})alkoxy” (x and y each being an integer) refers to an alkoxy group as defined before containing x to y carbon atoms. For example, a (C₁-C₃)alkoxy group contains from one to three carbon atoms.
The term “hydroxyalkoxy”, used alone or in combination, refers to a straight or branched chain alkoxy group containing from two to four carbon atoms wherein one of the carbon atoms bears a hydroxy group. The term “hydroxy(Cₓ-C_{y})alkoxy” (x and y each being an integer) refers to a hydroxyalkoxy group as defined before containing x to y carbon atoms. For example, a hydroxy(C₂-C₄)alkoxy group contains from two to four carbon atoms.
The term “halogen” refers to fluorine, chlorine, bromine or iodine, and preferably to fluorine or chlorine, and most preferably to fluorine.
The term “quinolone-resistant”, when used in this text, refers to a bacterial strain against which ciprofloxacin has a Minimal Inhibitory Concentration of 16 mg/L or higher (said Minimal Inhibitory Concentration being measured with the standard method described in “Methods for Dilution Antimicrobial Susceptibility Tests for Bacteria that Grow Aerobically”, Approved standard, 7^{th} ed., Clinical and Laboratory Standards Institute (CLSI) Document M7-A7, Wayne, Pa., USA (2006)).
The term “carbapenem-resistant”, when used in this text, refers to a bacterial strain against which imipenem has a Minimal Inhibitory Concentration of 16 mg/L or higher (said Minimal Inhibitory Concentration being measured with the standard method described in “Methods for Dilution Antimicrobial Susceptibility Tests for Bacteria that Grow Aerobically”, Approved standard, 7th ed., Clinical and Laboratory Standards Institute (CLSI) Document M7-A7, Wayne, Pa., USA (2006)).
The term “multi-drug resistant”, when used in this text, refers to a bacterial strain against which at least three antibiotic compounds selected from three distinct antibiotic categories have Minimal Inhibitory Concentrations (MICs) over their respective clinical breakpoints, whereby said three distinct antibiotic categories are chosen among penicillins, combinations of penicillins with beta-lactamase inhibitors, cephalosporins, carbapenems, monobactams, fluoro-quinolones, aminoglycosides, phosphonic acids, tetracyclins and polymixins. Clinical breakpoints are defined according to the latest available list published by Clinical and Laboratory Standards Institute (Wayne, Pa., USA). Accordingly, clinical breakpoints are the levels of MIC at which, at a given time, a bacterium is deemed either susceptible or resistant to treatment by the corresponding antibiotic or antibiotic combination.

Any reference hereinbefore or hereinafter to a compound of formula I of the present invention is to be understood as referring also to salts, especially pharmaceutically acceptable salts, of such a compound of formula I, as appropriate and expedient.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example ‘Handbook of Pharmaceutical Salts. Properties, Selection and Use.’, P. Heinrich Stahl, Camille G. Wermuth (Eds.), Wiley-VCH (2008) and ‘Pharmaceutical Salts and Co-crystals’, Johan Wouters and Luc Quéré (Eds.), RSC Publishing (2012).

In the text, it is understood that compounds of formula I wherein X represents sulphur have the formula indicated below:

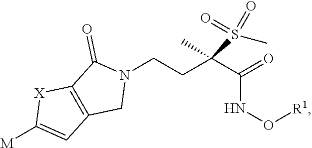

and compounds of formula I wherein X represents CH═CH have the formula indicated below:

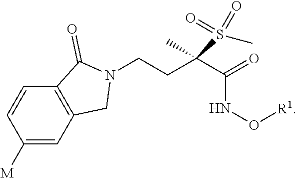

In this text, a bond interrupted by a wavy line shows a point of attachment of the radical drawn to the rest of the molecule. For example, the radical drawn below

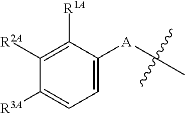

wherein A represents a bond, R^{1A} and R^{2A} both represent H and R^{3A} represents 1,2-dihydroxyethyl is the 4-(1,2-dihydroxyethyl)-phenyl group.

Besides, the term “room temperature” as used herein refers to a temperature of 25° C.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., and preferably to an interval extending from Y minus 5° C. to Y plus 5° C.

2) The invention notably relates to compounds of formula I according to embodiment 1) that are also compounds of formula Ip

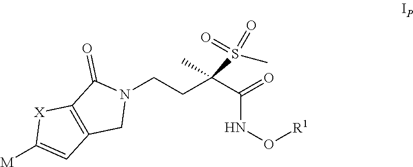

wherein

X represents sulphur or CH═CH;

R¹ represents H, PO₃H₂, SO₃H, phosphonooxymethyl or the group L represented below

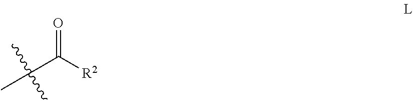

wherein

R² represents (C₁-C₄)alkylamino(C₁-C₄)alkyl, di(C₁-C₄)alkylamino(C₁-C₄)alkyl, phosphonooxy(C₁-C₄)alkyl, phosphonooxymethoxy, 2-(phosphonooxy-(C₁-C₄)alkyl)-phenyl, [2-(phosphonooxy-(C₁-C₄)alkyl)-phenyl]-(C₁-C₄)alkyl, or (2-(phosphonooxy)-phenyl)-(C₁-C₄)alkyl (especially 2-(2-(phosphonooxy)-phenyl)-ethyl);

M is one of the groups M^{A} and M^{B} represented below

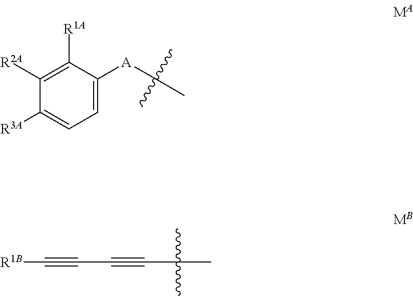

wherein A represents a bond or C≡C;

R^{1A} represents H or halogen (if halogen, then especially fluorine);

R^{2A} represents H or halogen;

R^{3A} represents (C₁-C₃)alkoxy (especially methoxy), hydroxy(C₂-C₄)alkoxy, hydroxy(C₁-C₄)alkyl (especially 1-hydroxy-2-methylpropan-2-yl), 1,2-dihydroxyethyl, (3-fluoroazetidin-1-yl)methyl, 3-fluoro-1-(oxetan-3-yl)azetidin-3-yl, 3-fluoro-1-methyl-azetidin-3-yl, (4-hydroxy-3-fluoropiperidin-1-yl)methyl, (4-hydroxy-3,3-difluoropiperidin-1-yl)methyl, (3-hydroxyazetidin-1-yl)methyl, 3-(w-hydroxy(C₂-C₄)alkyl)-azetidin-1-yl, or (4-hydroxypiperidin-1-yl)methyl;

R^{1B} represents amino(C₁-C₃)alkyl, 1-amino-cycloprop-1-yl, trans-2-(2-dimethylaminoacetoxymethyl)-cycloprop-1-yl, 1-(2-dimethylaminoacetoxymethyl)-cycloprop-1-yl, 1-(3-hydroxyazetidine)-1-carbonyloxymethyl, 1-hydroxymethyl-cycloprop-1-yl, trans-2-hydroxymethyl-cycloprop-1-yl, 1-fluoro-2-hydroxymethyl-cycloprop-1-yl, 2-fluoro-2-hydroxymethyl-cycloprop-1-yl, 1-{[(2-(phosphonooxy-(C₁-C₄)alkyl)-phenyl)-(C₁-C₄)alkyl]carbonyloxymethyl}cycloprop-1-yl, 1-{[2-(phosphonooxy-(C₁-C₄)alkyl)-phenyl]carbonyloxymethyl}cycloprop-1-yl, 1-{[(2-phosphonooxy-phenyl)-(C₁-C₄)alkyl]carbonyloxymethyl}cycloprop-1-yl, trans-2-{[(2-(phosphonooxy-(C₁-C₄)alkyl)-phenyl)-(C₁-C₄)alkyl]carbonyloxymethyl}cycloprop-1-yl, trans-2-{[2-(phosphonooxy-(C₁-C₄)alkyl)-phenyl]carbonyloxymethyl}cycloprop-1-yl, trans-2-{[(2-phosphonooxy-phenyl)-(C₁-C₄)alkyl]carbonyloxymethyl}cycloprop-1-yl, 2-fluoro-2-{[(2-(phosphonooxy-(C₁-C₄)alkyl)-phenyl)-(C₁-C₄)alkyl]carbonyloxymethyl}cycloprop-1-yl, 2-fluoro-2-{[2-(phosphonooxy-(C₁-C₄)alkyl)-phenyl]carbonyloxymethyl}cycloprop-1-yl, 2-fluoro-2-{[(2-phosphonooxy-phenyl)-(C₁-C₄)alkyl]carbonyloxymethyl}cycloprop-1-yl, 2-fluoro-2-(phosphonooxymethyl)-cycloprop-1-yl, 1-methyl-2-hydroxymethyl-cycloprop-1-yl, 2-hydroxymethyl-2-methylcycloprop-1-yl, 1-(2-hydroxyacetyl)-azetidin-3-yl, trans-(cis-3,4-dihydroxy)-cyclopent-1-yl, 2-(1,2-dihydroxyethyl)cycloprop-1-yl, 1-(dimethylamino)cycloprop-1-yl, 2-(morpholinomethyl)cycloprop-1-yl, N—(C₁-C₄)alkyl-azetidin-3-yl (especially N-methylazetidin-3-yl), N—(C₃-C₆)cycloalkyl-azetidin-3-yl, 3-fluoro-1-methyl-azetidin-3-yl, 1-(methylamino)cycloprop-1-yl, N-(ω-hydroxy(C₂-C₄)alkyl)-azetidin-3-yl (especially N-(2-hydroxyethyl)azetidin-3-yl), 4-fluoro-1-methylpiperidin-4-yl, or 1-(oxetan-3-yl)-azetidin-3-yl;

and to salts (in particular pharmaceutically acceptable salts) of such compounds of formula I_{P}.

3) The invention in particular relates to compounds of formula I according to embodiment 1) that are also compounds of formula I_{CE}

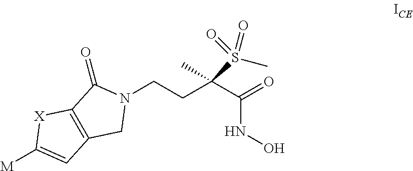

wherein

X represents sulphur or CH═CH;

M is one of the groups M^{A} and M^{B} represented below

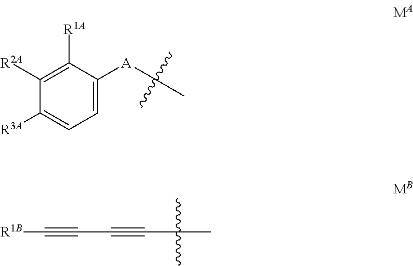

wherein A represents a bond or C≡C;

R^{1A} represents H or halogen (if halogen, then especially fluorine);

R^{2A} represents H;

R^{3A} represents H, (C₁-C₃)alkoxy (especially methoxy), hydroxy(C₁-C₄)alkyl (especially 1-hydroxy-2-methylpropan-2-yl), (3-fluoroazetidin-1-yl)methyl, 1-(oxetan-3-yl)azetidin-3-yl or 1-(oxetan-3-ylmethyl)azetidin-3-yl;

R^{1B} represents 1-amino-cycloprop-1-yl, trans-2-(2-dimethylaminoacetoxymethyl)-cycloprop-1-yl, 1-(3-hydroxyazetidine)-1-carbonyloxymethyl, 1-hydroxymethyl-cycloprop-1-yl, trans-2-hydroxymethyl-cycloprop-1-yl, 2-fluoro-2-hydroxymethyl-cycloprop-1-yl, 2-fluoro-2-(phosphonooxymethyl)-cycloprop-1-yl, trans-(cis-3,4-dihydroxy)-cyclopent-1-yl, 2-(1,2-dihydroxyethyl)cycloprop-1-yl, 2-((3-fluoroazetidin-1-yl)methyl)cyclopropyl, N—(C₁-C₄)alkyl-azetidin-3-yl (especially N-methylazetidin-3-yl), 1-(methylamino)cycloprop-1-yl, N-(ω-hydroxy(C₂-C₄)alkyl)-azetidin-3-yl (especially N-(2-hydroxyethyl)azetidin-3-yl), 2-(hydroxymethyl)-1-methylazetidin-3-yl, N-(2-hydroxy-2-methylpropyl)azetidin-3-yl, N-(ω-halo(C₂-C₄)alkyl)-azetidin-3-yl, 1-(3-hydroxypropyloxycarbonyl)-azetidin-3-yl, 2-(fluoromethyl)-1-methylazetidin-3-yl, N-(3-hydroxycyclobutyl)azetidin-3-yl, N-(oxetan-3-ylmethyl)azetidin-3-yl, N-(3-hydroxyoxetan-3-ylmethyl)azetidin-3-yl, N-(tetrahydrofuran-3-yl)azetidin-3-yl, N-(tetrahydro-2H-pyran-4-yl)azetidin-3-yl, trans-2-hydroxymethyl-1-methylazetidin-4-yl, (5-hydroxymethyl)-1-methylpyrrolidin-3-yl or 1-(oxetan-3-yl)-azetidin-3-yl;

and to salts (in particular pharmaceutically acceptable salts) of such compounds of formula ICE.

4) Another embodiment of the invention relates to compounds of formula I according to one of embodiments 1) to 3), wherein X represents sulphur.

5) Another embodiment of the invention relates to compounds of formula I according to one of embodiments 1) to 3), wherein X represents CH═CH.

6) Another embodiment of the invention relates to compounds of formula I according to any one of embodiments 1), 2), 4) and 5), wherein R¹ represents H.

7) Another embodiment of the invention relates to compounds of formula I according to any one of embodiments 1) to 6), wherein M represents the group M^{A}.

8) Another embodiment of the invention relates to compounds of formula I according to embodiment 7), wherein A represents a bond.

9) Another embodiment of the invention relates to compounds of formula I according to embodiment 7), wherein A represents C≡C.

10) Another embodiment of the invention relates to compounds of formula I according to any one of embodiments 7) to 9), wherein R^{1A} represents H.

11) Another embodiment of the invention relates to compounds of formula I according to any one of embodiments 7) to 9), wherein R^{1A} represents halogen.

12) Another embodiment of the invention relates to compounds of formula I according to embodiment 11), wherein R^{1A} represents fluorine.

13) Another embodiment of the invention relates to compounds of formula I according to any one of embodiments 7) to 12), wherein R^{2A} represents H.

14) Another embodiment of the invention relates to compounds of formula I according to any one of embodiments 7) to 13), wherein R^{3A} represents (C₁-C₃)alkoxy (especially methoxy), 1-(oxetan-3-yl)azetidin-3-yl, 1-(oxetan-3-ylmethyl)azetidin-3-yl or (3-fluoroazetidin-1-yl)methyl (and in particular wherein R^{3A} represents (C₁-C₃)alkoxy (especially methoxy) or (3-fluoroazetidin-1-yl)methyl).

15) Another embodiment of the invention relates to compounds of formula I according to any one of embodiments 7) to 13), wherein R^{3A} represents hydroxy(C₁-C₄)alkyl (especially 1-hydroxy-2-methylpropan-2-yl).

16) Another embodiment of the invention relates to compounds of formula I according to any one of embodiments 1) to 6), wherein M represents the group M^{B}.

17) Another embodiment of the invention relates to compounds of formula I according to embodiment 16), wherein R^{1B} represents 1-amino-cycloprop-1-yl, trans-2-(2-dimethylaminoacetoxymethyl)-cycloprop-1-yl, cis-2-fluoro-2-hydroxymethyl-cycloprop-1-yl, cis-2-fluoro-2-(phosphonooxymethyl)-cycloprop-1-yl, 1-hydroxymethyl-cycloprop-1-yl, trans-2-hydroxymethyl-cycloprop-1-yl, or N—(C₁-C₄)alkyl-azetidin-3-yl (especially N-methylazetidin-3-yl).

18) Another embodiment of the invention relates to compounds of formula I according to embodiment 16), wherein R^{1B} represents trans-(cis-3,4-dihydroxy)-cyclopent-1-yl, 1-(3-hydroxyazetidine)-1-carbonyloxymethyl, N-(ω-hydroxy(C₂-C₄)alkyl)-azetidin-3-yl (especially N-(2-hydroxyethyl)azetidin-3-yl), or 1-(methylamino)cycloprop-1-yl.

19) Yet another embodiment of the invention relates to compounds of formula I according to embodiment 16), wherein R^{1B} represents 2-(hydroxymethyl)-1-methylazetidin-3-yl, N-(2-hydroxy-2-methylpropyl)azetidin-3-yl, N-(ω-halo(C₂-C₄)alkyl)-azetidin-3-yl, 1-(3-hydroxypropyloxycarbonyl)-azetidin-3-yl, N-(3-hydroxycyclobutyl)azetidin-3-yl or N-(tetrahydro-2H-pyran-4-yl)azetidin-3-yl.

20) Preferably, the compounds of formula I according to embodiment 19) will be such that R^{1B} represents N-(ω-halo(C₂-C₄)alkyl)-azetidin-3-yl, N-(3-hydroxycyclobutyl)azetidin-3-yl or N-(tetrahydro-2H-pyran-4-yl)azetidin-3-yl (and in particular such that R^{1B} represents N-(2-fluoroethyl)-azetidin-3-yl, N-(3-hydroxycyclobutyl)azetidin-3-yl or N-(tetrahydro-2H-pyran-4-yl)azetidin-3-yl).

21) Another embodiment of the invention relates to compounds of formula I according to embodiment 1) or 2), wherein:

X represents sulphur or CH═CH;

R¹ represents H;

M represents M^{A} or M^{B},

wherein A represents a bond or C≡C,

R^{1A} represents H or halogen (if halogen, then especially fluorine),

R^{2A} represents H, and

R^{3A} represents (C₁-C₃)alkoxy (especially methoxy), (3-fluoroazetidin-1-yl)methyl or hydroxy(C₁-C₄)alkyl (especially 1-hydroxy-2-methylpropan-2-yl);

and wherein R^{1B} represents 1-amino-cycloprop-1-yl, trans-(cis-3,4-dihydroxy)-cyclopent-1-yl, trans-2-(2-dimethylaminoacetoxymethyl)-cycloprop-1-yl, cis-2-fluoro-2-hydroxymethyl-cycloprop-1-yl, cis-2-fluoro-2-(phosphonooxymethyl)-cycloprop-1-yl, 1-(3-hydroxyazetidine)-1-carbonyloxymethyl, N-(ω-hydroxy(C₂-C₄)alkyl)-azetidin-3-yl (especially N-(2-hydroxyethyl)azetidin-3-yl), 1-hydroxymethyl-cycloprop-1-yl, trans-2-hydroxymethyl-cycloprop-1-yl, 1-(methylamino)cycloprop-1-yl, N—(C₁-C₄)alkyl-azetidin-3-yl (especially N-methylazetidin-3-yl), or 1-(oxetan-3-yl)-azetidin-3-yl.

22) A further embodiment of the invention relates to compounds of formula I according to embodiment 1) or 2), wherein:

X represents CH═CH;

R¹ represents H;

M represents the group M^{B},

wherein R^{1B} represents 1-amino-cycloprop-1-yl or 1-hydroxymethyl-cycloprop-1-yl.

23) Another embodiment of the invention relates to compounds of formula I according to embodiment 1) or 2), wherein:

X represents sulphur;

R¹ represents H; and

M represents the group M^{A} or M^{B},

wherein A represents a bond or C≡C,

R^{1A} represents H or fluorine,

R^{2A} represents H,

R^{3A} represents (C₁-C₃)alkoxy (especially methoxy) or (3-fluoroazetidin-1-yl)methyl, and R^{1B} represents 1-amino-cycloprop-1-yl, trans-2-(2-dimethylaminoacetoxymethyl)-cycloprop-1-yl, trans-2-hydroxymethyl-cycloprop-1-yl, 2-fluoro-2-hydroxymethyl-cycloprop-1-yl, 2-fluoro-2-(phosphonooxymethyl)-cycloprop-1-yl, 2-(1,2-dihydroxyethyl)cycloprop-1-yl, N—(C₁-C₄)alkyl-azetidin-3-yl (especially N-methylazetidin-3-yl), N-(ω-halo(C₂-C₄)alkyl)-azetidin-3-yl, 2-(fluoromethyl)-1-methylazetidin-3-yl, N-(3-hydroxycyclobutyl)azetidin-3-yl, N-(tetrahydrofuran-3-yl)azetidin-3-yl or N-(tetrahydro-2H-pyran-4-yl)azetidin-3-yl.

24) One sub-embodiment of the invention relates to compounds of formula I according to embodiment 23), wherein:

X represents sulphur;

R¹ represents H; and

M represents the group M^{A},

wherein A represents a bond or C≡C,

R^{1A} represents H or fluorine,

R^{2A} represents H, and

R^{3A} represents (C₁-C₃)alkoxy (especially methoxy) or (3-fluoroazetidin-1-yl)methyl.

25) Another sub-embodiment of the invention relates to compounds of formula I according to embodiment 23), wherein:

X represents sulphur;

R¹ represents H; and

M represents the group M^{B},

wherein R^{1B} represents 1-amino-cycloprop-1-yl, trans-2-(2-dimethylaminoacetoxymethyl)-cycloprop-1-yl, trans-2-hydroxymethyl-cycloprop-1-yl, 2-fluoro-2-hydroxymethyl-cycloprop-1-yl, 2-fluoro-2-(phosphonooxymethyl)-cycloprop-1-yl, 2-(1,2-dihydroxyethyl)cycloprop-1-yl, N—(C₁-C₄)alkyl-azetidin-3-yl (especially N-methylazetidin-3-yl), N-(ω-halo(C₂-C₄)alkyl)-azetidin-3-yl, 2-(fluoromethyl)-1-methylazetidin-3-yl, N-(3-hydroxycyclobutyl)azetidin-3-yl, N-(tetrahydrofuran-3-yl)azetidin-3-yl or N-(tetrahydro-2H-pyran-4-yl)azetidin-3-yl.

26) Another embodiment of the invention relates to compounds of formula I as defined in one of embodiments 1) to 25) which are isotopically labelled, especially ²H (deuterium) labelled compounds of formula I, which compounds are identical to the compounds of formula I as defined in one of embodiments 1) to 25) except that one or more atoms has or have each been replaced by an atom having the same atomic number but an atomic mass different from the atomic mass usually found in nature. Isotopically labelled, especially ²H (deuterium) labelled compounds of formula I and salts (in particular pharmaceutically acceptable salts) thereof are thus within the scope of the present invention. Substitution of hydrogen with the heavier isotope ²H (deuterium) may lead to greater metabolic stability, resulting e.g. in an increased in-vivo half-life, reduced dosage requirements, or an improved safety profile. In one variant of the invention, the compounds of formula I are not isotopically labelled, or they are labelled only with one or more deuterium atoms. Isotopically labelled compounds of formula I may be prepared in analogy to the methods described hereinafter, but using the appropriate isotopic variation of suitable reagents or starting materials.

27) Another embodiment of the invention relates to a compound of formula I according to embodiment 1) or 2) selected from the group consisting of:
(2R)—N-hydroxy-4-(2-((4-(1-hydroxy-2-methylpropan-2-yl)phenyl)ethynyl)-6-oxo-4,6-dihydro-5H-thieno[2,3-c]pyrrol-5-yl)-2-methyl-2-(methylsulfonyl)butanamide; and
(2R)—N-hydroxy-4-(2-((1-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)butanamide;

and to salts (in particular the pharmaceutically acceptable salts) of such compounds.

28) Another embodiment of the invention relates to a compound of formula I according to embodiment 1) or 2) selected from the group consisting of:
(2R)—N-hydroxy-4-(2-(((1R,2R)-2-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)butanamide;
(2R)-4-(2-(2-fluoro-4-methoxyphenyl)-6-oxo-4,6-dihydro-5H-thieno[2,3-c]pyrrol-5-yl)-N-hydroxy-2-methyl-2-(methylsulfonyl)butanamide;
(2R)-4-(2-((1-aminocyclopropyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide;
(2R)-4-(2-(((1R,2R)-2-fluoro-2-(hydroxymethyl)cyclopropyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide;
(2R)—N-hydroxy-2-methyl-4-(2-((1-(methylamino)cyclopropyl)buta-1,3-diyn-1-yl)-6-oxo-4,6-dihydro-5H-thieno[2,3-c]pyrrol-5-yl)-2-(methylsulfonyl)butanamide;
(3R)-5-(5-(4-(hydroxyamino)-3-methyl-3-(methylsulfonyl)-4-oxobutyl)-6-oxo-5,6-dihydro-4H-thieno[2,3-c]pyrrol-2-yl)penta-2,4-diyn-1-yl 3-hydroxyazetidine-1-carboxylate;
(2R)—N-hydroxy-2-methyl-4-(5-((1-(methylamino)cyclopropyl)buta-1,3-diyn-1-yl)-1-oxoisoindolin-2-yl)-2-(methylsulfonyl)butanamide;
(2R)—N-hydroxy-2-methyl-4-(5-((1-methylazetidin-3-yl)buta-1,3-diyn-1-yl)-1-oxoisoindolin-2-yl)-2-(methylsulfonyl)butanamide;
(3R)-5-(2-(4-(hydroxyamino)-3-methyl-3-(methylsulfonyl)-4-oxobutyl)-1-oxoisoindolin-5-yl)penta-2,4-diyn-1-yl 3-hydroxyazetidine-1-carboxylate;
(2R)-4-(5-(2-fluoro-4-methoxyphenyl)-1-oxoisoindolin-2-yl)-N-hydroxy-2-methyl-2-(methylsulfonyl)butanamide;
(2R)—N-hydroxy-2-methyl-4-(2-((1-methylazetidin-3-yl)buta-1,3-diyn-1-yl)-6-oxo-4,6-dihydro-5H-thieno[2,3-c]pyrrol-5-yl)-2-(methylsulfonyl)butanamide;
(2R)—N-hydroxy-4-(5-((1-(hydroxymethyl)cyclopropyl)buta-1,3-diyn-1-yl)-1-oxoisoindolin-2-yl)-2-methyl-2-(methylsulfonyl)butanamide;
(2R)-4-(5-((1-aminocyclopropyl)buta-1,3-diyn-1-yl)-1-oxoisoindolin-2-yl)-N-hydroxy-2-methyl-2-(methylsulfonyl)butanamide;
(2R)-4-(2-((4-((3-fluoroazetidin-1-yl)methyl)phenyl)ethynyl)-6-oxo-4,6-dihydro-5H-thieno[2,3-c]pyrrol-5-yl)-N-hydroxy-2-methyl-2-(methylsulfonyl)butanamide;
(2R)—N-hydroxy-2-methyl-2-(methylsulfonyl)-4-(2-((1-(oxetan-3-yl)azetidin-3-yl)buta-1,3-diyn-1-yl)-6-oxo-4,6-dihydro-5H-thieno[2,3-c]pyrrol-5-yl)butanamide;
((1R,2R)-1-fluoro-2-((5-((3R)-4-(hydroxyamino)-3-methyl-3-(methylsulfonyl)-4-oxobutyl)-6-oxo-5,6-dihydro-4H-thieno[2,3-c]pyrrol-2-yl)buta-1,3-diyn-1-yl)cyclopropyl)methyl dihydrogen phosphate;
(2R)—N-hydroxy-4-(2-((1-(2-hydroxyethyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)-4-(5-(((1R,2R)-2-fluoro-2-(hydroxymethyl)cyclopropyl)buta-1,3-diyn-1-yl)-1-oxoisoindolin-2-yl)-N-hydroxy-2-methyl-2-(methylsulfonyl)butanamide;
(2R)—N-hydroxy-4-(2-(((1S,2S)-2-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)butanamide;
((1S,2S)-2-((5-((3R)-4-(hydroxyamino)-3-methyl-3-(methylsulfonyl)-4-oxobutyl)-6-oxo-5,6-dihydro-4H-thieno[2,3-c]pyrrol-2-yl)buta-1,3-diyn-1-yl)cyclopropyl)methyl dimethylglycinate; and
(2R)-4-(2-(((1S,3R,4S)-3,4-dihydroxycyclopentyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide; and to salts (in particular the pharmaceutically acceptable salts) of such compounds.

29) Yet another embodiment of the invention relates to a compound of formula I according to embodiment 1) selected from the group consisting of:
(2R)-4-(2-(((1S,2S)-2-((2R)-1,2-dihydroxyethyl)cyclopropyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide;
(2R)-4-(2-((1-(2-fluoroethyl)azetidin-3-yl)buta-1,3-diyn-1-yl)-6-oxo-4,6-dihydro-5H-thieno[2,3-c]pyrrol-5-yl)-N-hydroxy-2-methyl-2-(methylsulfonyl)butanamide;
(R)—N-hydroxy-4-(2-((1-isopropylazetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-4-(2-((1-(2-hydroxy-2-methylpropyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-2-methyl-2-(methylsulfonyl)-4-(2-((4-(1-(oxetan-3-yl)azetidin-3-yl)phenyl)ethynyl)-6-oxo-4,6-dihydro-5H-thieno[2,3-c]pyrrol-5-yl)butanamide;
(2R)—N-hydroxy-2-methyl-2-(methylsulfonyl)-4-(6-oxo-2-((1-(tetrahydro-2H-pyran-4-yl)azetidin-3-yl)buta-1,3-diyn-1-yl)-4,6-dihydro-5H-thieno[2,3-c]pyrrol-5-yl)butanamide;
(2R)—N-hydroxy-2-methyl-2-(methylsulfonyl)-4-(2-((1-(oxetan-3-ylmethyl)azetidin-3-yl)buta-1,3-diyn-1-yl)-6-oxo-4,6-dihydro-5H-thieno[2,3-c]pyrrol-5-yl)butanamide;
(2R)—N-hydroxy-2-methyl-2-(methylsulfonyl)-4-(2-((4-(1-(oxetan-3-yl)azetidin-3-yl)phenyl)ethynyl)-6-oxo-4,6-dihydro-5H-thieno[2,3-c]pyrrol-5-yl)butanamide;
(2R)-4-(2-(((1S,2S)-2-((3-fluoroazetidin-1-yl)methyl)cyclopropyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide;
(2R)—N-hydroxy-4-(2-((1-((1s,3s)-3-hydroxycyclobutyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-4-(2-((1-((1r,3r)-3-hydroxycyclobutyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-4-(5-((4-(2-hydroxyethyl)phenyl)ethynyl)-1-oxoisoindolin-2-yl)-2-methyl-2-(methylsulfonyl)butanamide;
3-hydroxypropyl (2R)-3-((5-(4-(hydroxyamino)-3-methyl-3-(methylsulfonyl)-4-oxobutyl)-6-oxo-5,6-dihydro-4H-thieno[2,3-c]pyrrol-2-yl)buta-1,3-diyn-1-yl)azetidine-1-carboxylate;
(2R)—N-hydroxy-4-(2-(((2R,3R)-2-(hydroxymethyl)-1-methylazetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-4-(2-(((3R,5R)-5-(hydroxymethyl)-1-methylpyrrolidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-2-methyl-2-(methylsulfonyl)-4-(6-oxo-2-((1-((3R)-tetrahydrofuran-3-yl)azetidin-3-yl)buta-1,3-diyn-1-yl)-4,6-dihydro-5H-thieno[2,3-c]pyrrol-5-yl)butanamide;
(2R)—N-hydroxy-2-methyl-2-(methylsulfonyl)-4-(6-oxo-2-((1-((3S)-tetrahydrofuran-3-yl)azetidin-3-yl)buta-1,3-diyn-1-yl)-4,6-dihydro-5H-thieno[2,3-c]pyrrol-5-yl)butanamide;
(2R)—N-hydroxy-4-(2-((1-((3-hydroxyoxetan-3-yl)methyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-4-(2-(((2R,4R)-4-(hydroxymethyl)-1-methylazetidin-2-yl)buta-1,3-diyn-1-yl)-6-oxo-4,6-dihydro-5H-thieno[2,3-c]pyrrol-5-yl)-2-methyl-2-(methylsulfonyl)butanamide;
(2R)-4-(2-(((2S,3S)-2-(fluoromethyl)-1-methylazetidin-3-yl)buta-1,3-diyn-1-yl)-6-oxo-4,6-dihydro-5H-thieno[2,3-c]pyrrol-5-yl)-N-hydroxy-2-methyl-2-(methylsulfonyl)butanamide; and
(2R)—N-hydroxy-2-methyl-2-(methylsulfonyl)-4-(6-oxo-2-(phenylethynyl)-4,6-dihydro-5H-thieno[2,3-c]pyrrol-5-yl)butanamide;

and to salts (in particular the pharmaceutically acceptable salts) of such compounds.

30) The invention further relates to the compounds of formula I which are selected from the group consisting of the compounds listed in embodiment 27), the compounds listed in embodiment 28) and the compounds listed in embodiment 29) (and notably to the compounds of formula I which are selected from the group consisting of the compounds listed in embodiment 27) and the compounds listed in embodiment 28)). In particular, it also relates to the groups of compounds of formula I selected from the group consisting of the compounds listed in embodiment 27), the compounds listed in embodiment 28) and the compounds listed in embodiment 29), which groups of compounds furthermore correspond to one of embodiments 1) to 25), as well as to the salts (in particular the pharmaceutically acceptable salts) of such compounds (and notably to the compounds of formula I which are selected from the group consisting of the compounds listed in embodiment 27) and the compounds listed in embodiment 28), which groups of compounds furthermore correspond to embodiment 2) taken in combination with any one of embodiments 4) to 18) and 21)). The invention moreover relates to any individual compound of formula I selected from the group consisting of the compounds listed in embodiment 27), the compounds listed in embodiment 28) and the compounds listed in embodiment 29), and to the salts (in particular the pharmaceutically acceptable salts) of such individual compound.

The compounds of formula I according to this invention, i.e. according to one of embodiments 1) to 30) above, exhibit antibacterial activity in a biological environment (i.e. in the presence of a phosphatase, an esterase, a sulfatase or any suitable equivalent thereof capable of removing the group R¹ that is not hydrogen and capable of transforming if necessary the hydroxy prodrug groups when present in the group R^{1B} to the corresponding hydroxy group), especially against Gram-negative organisms and are therefore suitable to treat bacterial infections in mammals, especially humans. Said compounds may also be used for veterinary applications, such as treating infections in livestock and companion animals. They may further constitute substances for preserving inorganic and organic materials in particular all types of organic materials for example polymers, lubricants, paints, fibres, leather, paper and wood.

They may therefore be used for the treatment or prevention of infectious disorders caused by fermentative or non-fermentative Gram-negative bacteria, especially those caused by susceptible and multi-drug resistant Gram-negative bacteria. Examples of such Gram-negative bacteria include Acinetobacter spp. such as Acinetobacter baumannii or Acinetobacter haemolyticus, Actinobacillus actinomycetemcomitans, Achromobacter spp. such as Achromobacter xylosoxidans or Achromobacter faecalis, Aeromonas spp. such as Aeromonas hydrophila, Bacteroides spp. such as Bacteroides fragilis, Bacteroides theataioatamicron, Bacteroides distasonis, Bacteroides ovatus or Bacteroides vulgatus, Bartonella hensenae, Bordetella spp. such as Bordetella pertussis, Borrelia spp. such as Borrelia Burgdorferi, Brucella spp. such as Brucella melitensis, Burkholderia spp. such as Burkholderia cepacia, Burkholderia pseudomallei or Burkholderia mallei, Campylobacter spp. such as Campylobacter jejuni, Campylobacter fetus or Campylobacter coli, Cedecea, Chlamydia spp. such as Chlamydia pneumoniae, Chlamydia trachomatis, Citrobacter spp. such as Citrobacter diversus (koseri) or Citrobacter freundii, Coxiella burnetii, Edwardsiella spp. such as Edwarsiella tarda, Ehrlichia chafeensis, Eikenella corrodens, Enterobacter spp. such as Enterobacter cloacae, Enterobacter aerogenes, Enterobacter agglomerans, Escherichia coli, Francisella tularensis, Fusobacterium spp., Haemophilus spp. such as Haemophilus influenzae (beta-lactamase positive and negative) or Haemophilus ducreyi, Helicobacter pylori, Kingella kingae, Klebsiella spp. such as Klebsiella oxytoca, Klebsiella pneumoniae (including those encoding extended-spectrum beta-lactamases (hereinafter “ESBLs”), carbapenemases (KPCs), cefotaximase-Munich (CTX-M), metallo-beta-lactamases, and AmpC-type beta-lactamases that confer resistance to currently available cephalosporins, cephamycins, carbapenems, beta-lactams, and beta-lactam/beta-lactamase inhibitor combinations), Klebsiella rhinoscleromatis or Klebsiella ozaenae, Legionella pneumophila, Mannheimia haemolyticus, Moraxella catarrhalis (beta-lactamase positive and negative), Morganella morganii, Neisseria spp. such as Neisseria gonorrhoeae or Neisseria meningitidis, Pasteurella spp. such as Pasteurella multocida, Plesiomonas shigelloides, Porphyromonas spp. such as Porphyromonas asaccharolytica, Prevotella spp. such as Prevotella corporis, Prevotella intermedia or Prevotella endodontalis, Proteus spp. such as Proteus mirabilis, Proteus vulgaris, Proteus penneri or Proteus myxofaciens, Porphyromonas asaccharolytica, Plesiomonas shigelloides, Providencia spp. such as Providencia stuartii, Providencia rettgeri or Providencia alcalifaciens, Pseudomonas spp. such as Pseudomonas aeruginosa (including ceftazidime-, cefpirome- and cefepime-resistant P. aeruginosa, carbapenem-resistant P. aeruginosa or quinolone-resistant P. aeruginosa) or Pseudomonas fluorescens, Ricketsia prowazekii, Salmonella spp. such as Salmonella typhi or Salmonella paratyphi, Serratia marcescens, Shigella spp. such as Shigella flexneri, Shigella boydii, Shigella sonnei or Shigella dysenteriae, Streptobacillus moniliformis, Stenotrophomonas maltophilia, Treponema spp., Vibrio spp. such as Vibrio cholerae, Vibrio parahaemolyticus, Vibrio vulnificus, Vibrio alginolyticus, Yersinia spp. such as Yersinia enterocolitica, Yersinia pestis or Yersinia pseudotuberculosis.

The compounds of formula I according to this invention are thus useful for treating a variety of infections caused by fermentative or non-fermentative Gram-negative bacteria, especially infections such as: nosocomial pneumonia (related to infection by Legionella pneumophila, Haemophilus influenzae, or Chlamydia pneumoniae); urinary tract infections; systemic infections (bacteraemia and sepsis); skin and soft tissue infections (including burn patients); surgical infections; intraabdominal infections; lung infections (including those in patients with cystic fibrosis); Helicobacter pylori (and relief of associated gastric complications such as peptic ulcer disease, gastric carcinogenesis, etc.); endocarditis; diabetic foot infections; osteomyelitis; otitis media, sinusitus, bronchitis, tonsillitis, and mastoiditis related to infection by Haemophilus influenzae or Moraxella catarrhalis; pharynigitis, rheumatic fever, and glomerulonephritis related to infection by Actinobacillus haemolyticum; sexually transmitted diseases related to infection by Chlamydia trachormatis, Haemophilus ducreyi, Treponema pallidum, Ureaplasma urealyticum, or Neisseria gonorrheae; systemic febrile syndromes related to infection by Borrelia recurrentis; Lyme disease related to infection by Borrelia burgdorferi; conjunctivitis, keratitis, and dacrocystitis related to infection by Chlamydia trachomatis, Neisseria gonorrhoeae or H. influenzae; gastroenteritis related to infection by Campylobacter jejuni; persistent cough related to infection by Bordetella pertussis and gas gangrene related to infection by Bacteroides spp. Other bacterial infections and disorders related to such infections that may be treated or prevented in accord with the method of the present invention are referred to in J. P. Sanford et al., “The Sanford Guide to Antimicrobial Therapy”, 26th Edition, (Antimicrobial Therapy, Inc., 1996).

The preceding lists of infections and pathogens are to be interpreted merely as examples and in no way as limiting.

The compounds of formula I according to this invention, or the pharmaceutically acceptable salts thereof, may therefore be used for the preparation of a medicament, and are suitable, for the prevention or treatment of a bacterial infection, in particular for the prevention or treatment of a bacterial infection caused by Gram-negative bacteria, especially by multi-drug resistant Gram-negative bacteria.

The compounds of formula I according to this invention, or the pharmaceutically acceptable salts thereof, may thus especially be used for the preparation of a medicament, and are suitable, for the prevention or treatment of a bacterial infection caused by Gram-negative bacteria selected from the group consisting of Acinetobacter baumannii, Burkholderia spp. (e.g. Burkholderia cepacia), Citrobacter spp., Enterobacter aerogenes, Enterobacter cloacae, Escherichia coli, Klebsiella oxytoca, Klebsiella pneumoniae, Serratia marcescens, Stenotrophomonas maltophilia and Pseudomonas aeruginosa (notably for the prevention or treatment of a bacterial infection caused by Acinetobacter baumannii bacteria, Escherichia coli bacteria, Klebsiella pneumoniae bacteria or Pseudomonas aeruginosa bacteria, and in particular for the prevention or treatment of a bacterial infection mediated by quinolone-resistant Acinetobacter baumannii bacteria or quinolone-resistant Klebsiella pneumoniae bacteria).

The compounds of formula I according to this invention, or the pharmaceutically acceptable salts thereof, may more especially be used for the preparation of a medicament, and are suitable, for the prevention or treatment of a bacterial infection caused by Gram-negative bacteria selected from the group consisting of Citrobacter spp., Enterobacter aerogenes, Enterobacter cloacae, Escherichia coli, Klebsiella oxytoca, Klebsiella pneumoniae, Serratia marcescens, Stenotrophomonas maltophilia and Pseudomonas aeruginosa bacteria (notably of a bacterial infection caused by Gram-negative bacteria selected from the group consisting of Klebsiella pneumoniae and Pseudomonas aeruginosa bacteria, and in particular of a bacterial infection caused by Pseudomonas aeruginosa bacteria).

The compounds of formula I according to this invention, or the pharmaceutically acceptable salts thereof, may thus especially be used for the preparation of a medicament, and are suitable, for the prevention or treatment of a bacterial infection selected from urinary tract infections, systemic infections (such as bacteraemia and sepsis), skin and soft tissue infections (including burn patients), surgical infections; intraabdominal infections and lung infections (including those in patients with cystic fibrosis).

The compounds of formula I according to this invention, or the pharmaceutically acceptable salts thereof, may more especially be used for the preparation of a medicament, and are suitable, for the prevention or treatment of a bacterial infection selected from urinary tract infections, intraabdominal infections and lung infections (including those in patients with cystic fibrosis), and in particular for the prevention or treatment of a bacterial infection selected from urinary tract infections and intraabdominal infections.

Besides, the compounds of formula I according to this invention display antibacterial properties and have the ability to improve permeability of the outer membrane of Gram-negative bacteria to other antibacterial agents. Their use in combination with another antibacterial agent might offer some further advantages such as lowered side-effects of drugs due to lower doses used or shorter time of treatment, more rapid cure of infection shortening hospital stays, increasing spectrum of pathogens controlled, and decreasing incidence of development of resistance to antibiotics. The antibacterial agent for use in combination with a compound of formula I according to this invention will be selected from the group consisting of a penicillin antibiotic (such as ampicillin, piperacillin, penicillin G, amoxicillin, or ticarcillin), a cephalosporin antibiotic (such as ceftriaxone, cefatazidime, cefepime, cefotaxime) a carbapenem antibiotic (such as imipenem, or meropenem), a monobactam antibiotic (such as aztreonam), a fluoroquinolone antibiotic (such as ciprofloxacin, moxifloxacin or levofloxacin), a macrolide antibiotic (such as erythromycin or azithromycin), an aminoglycoside antibiotic (such as amikacin, gentamycin or tobramycin), a glycopeptide antibiotic (such as vancomycin or teicoplanin), a tetracycline antibiotic (such as tetracycline, oxytetracycline, doxycycline, minocycline or tigecycline), and linezolid, clindamycin, telavancin, daptomycin, novobiocin, rifampicin and polymyxin. Preferably, the antibacterial agent for use in combination with a compound of formula I according to this invention will be selected from the group consisting of vancomycin, tigecycline and rifampicin.

The compounds of formula I according to this invention, or the pharmaceutically acceptable salt thereof, may moreover be used for the preparation of a medicament, and are suitable, for the prevention or treatment (and especially the treatment) of infections caused by biothreat Gram negative bacterial pathogens as listed by the US Center for Disease Control (the list of such biothreat bacterial pathogens can be found at the web page http://www.selectagents.gov/SelectAgentsandToxinsList.html), and in particular by Gram negative pathogens selected from the group consisting of Yersinia pestis, Francisella tularensis (tularemia), Burkholderia pseudomallei and Burkholderia mallei.

One aspect of this invention therefore relates to the use of a compound of formula I according to one of embodiments 1) to 30), or of a pharmaceutically acceptable salt thereof, for the manufacture of a medicament for the prevention or treatment of a bacterial infection (in particular one of the previously mentioned infections caused by Gram-negative bacteria, especially by multi-drug resistant Gram-negative bacteria). Another aspect of this invention relates to a compound of formula I according to one of embodiments 1) to 30), or a pharmaceutically acceptable salt thereof, for the prevention or treatment of a bacterial infection (in particular for the prevention or treatment of one of the previously mentioned infections caused by Gram-negative bacteria, especially by multi-drug resistant Gram-negative bacteria). Yet another aspect of this invention relates to a compound of formula I according to one of embodiments 1) to 30), or a pharmaceutically acceptable salt thereof, as a medicament. Yet a further aspect of this invention relates to a pharmaceutical composition containing, as active ingredient, a compound of formula I according to one of embodiments 1) to 30), or a pharmaceutically acceptable salt thereof, and at least one therapeutically inert excipient.

As well as in humans, bacterial infections can also be treated using compounds of formula I (or pharmaceutically acceptable salts thereof) in other species like pigs, ruminants, horses, dogs, cats and poultry.

The present invention also relates to pharmacologically acceptable salts and to compositions and formulations of compounds of formula I.

A pharmaceutical composition according to the present invention contains at least one compound of formula I (or a pharmaceutically acceptable salt thereof) as the active ingredient and optionally carriers and/or diluents and/or adjuvants, and may also contain additional known antibiotics.

The compounds of formula I and their pharmaceutically acceptable salts can be used as medicaments, e.g. in the form of pharmaceutical compositions for enteral or parenteral administration.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula I or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Another aspect of the invention concerns a method for the prevention or the treatment of a Gram-negative bacterial infection in a patient, comprising the administration to said patient of a pharmaceutically active amount of a compound of formula I according to one of embodiments 1) to 30) or a pharmaceutically acceptable salt thereof. Accordingly, the invention provides a method for the prevention or the treatment of a bacterial infection caused by Gram-negative bacteria (notably for the prevention or treatment of a bacterial infection caused by Acinetobacter baumannii bacteria, Escherichia coli bacteria, Klebsiella pneumoniae bacteria or Pseudomonas aeruginosa bacteria, and in particular for the prevention or treatment of a bacterial infection caused by quinolone-resistant Acinetobacter baumannii bacteria or quinolone-resistant Klebsiella pneumoniae bacteria) in a patient, comprising the administration to said patient of a pharmaceutically active amount of a compound of formula I according to one of embodiments 1) to 30) or a pharmaceutically acceptable salt thereof.

Moreover, the compounds of formula I according to this invention may also be used for cleaning purposes, e.g. to remove pathogenic microbes and bacteria from surgical instruments, catheters and artificial implants or to make a room or an area aseptic. For such purposes, the compounds of formula I could be contained in a solution or in a spray formulation.

This invention, thus, relates to the compounds of formula I as defined in embodiment 1), or further limited under consideration of their respective dependencies by the characteristics of any one of embodiments 2) to 30), and to pharmaceutically acceptable salts thereof. It relates furthermore to the use of such compounds as medicaments, especially for the prevention or treatment of a bacterial infection, in particular for the prevention or treatment of a bacterial infection caused by Gram-negative bacteria (notably for the prevention or treatment of a bacterial infection caused by Acinetobacter baumannii bacteria, Escherichia coli bacteria, Klebsiella pneumoniae bacteria or Pseudomonas aeruginosa bacteria, and in particular for the prevention or treatment of a bacterial infection caused by quinolone-resistant Acinetobacter baumannii bacteria or quinolone-resistant Klebsiella pneumoniae bacteria). The following embodiments relating to the compounds of formula I according to embodiment 1) are thus possible and intended and herewith specifically disclosed in individualised form:

1, 2+1, 3+1, 4+1, 4+2+1, 4+3+1, 5+1, 5+2+1, 5+3+1, 6+1, 6+2+1, 6+4+1, 6+4+2+1, 6+4+3+1, 6+5+1, 6+5+2+1, 6+5+3+1, 7+1, 7+2+1, 7+3+1, 7+4+1, 7+4+2+1, 7+4+3+1, 7+5+1, 7+5+2+1, 7+5+3+1, 7+6+1, 7+6+2+1, 7+6+4+1, 7+6+4+2+1, 7+6+4+3+1, 7+6+5+1, 7+6+5+2+1, 7+6+5+3+1, 8+7+1, 8+7+2+1, 8+7+3+1, 8+7+4+1, 8+7+4+2+1, 8+7+4+3+1, 8+7+5+1, 8+7+5+2+1, 8+7+5+3+1, 8+7+6+1, 8+7+6+2+1, 8+7+6+4+1, 8+7+6+4+2+1, 8+7+6+4+3+1, 8+7+6+5+1, 8+7+6+5+2+1, 8+7+6+5+3+1, 9+7+1, 9+7+2+1, 9+7+3+1, 9+7+4+1, 9+7+4+2+1, 9+7+4+3+1, 9+7+5+1, 9+7+5+2+1, 9+7+5+3+1, 9+7+6+1, 9+7+6+2+1, 9+7+6+4+1, 9+7+6+4+2+1, 9+7+6+4+3+1, 9+7+6+5+1, 9+7+6+5+2+1, 9+7+6+5+3+1, 10+1, 10+2+1, 10+7+1, 10+7+2+1, 10+7+3+1, 10+7+4+1, 10+7+4+2+1, 10+7+4+3+1, 10+7+5+1, 10+7+5+2+1, 10+7+5+3+1, 10+7+6+1, 10+7+6+2+1, 10+7+6+4+1, 10+7+6+4+2+1, 10+7+6+4+3+1, 10+7+6+5+1, 10+7+6+5+2+1, 10+7+6+5+3+1, 10+8+7+1, 10+8+7+2+1, 10+8+7+3+1, 10+8+7+4+1, 10+8+7+4+2+1, 10+8+7+4+3+1, 10+8+7+5+1, 10+8+7+5+2+1, 10+8+7+5+3+1, 10+8+7+6+1, 10+8+7+6+2+1, 10+8+7+6+4+1, 10+8+7+6+4+2+1, 10+8+7+6+4+3+1, 10+8+7+6+5+1, 10+8+7+6+5+2+1, 10+8+7+6+5+3+1, 10+9+7+1, 10+9+7+2+1, 10+9+7+3+1, 10+9+7+4+1, 10+9+7+4+2+1, 10+9+7+4+3+1, 10+9+7+5+1, 10+9+7+5+2+1, 10+9+7+5+3+1, 10+9+7+6+1, 10+9+7+6+2+1, 10+9+7+6+4+1, 10+9+7+6+4+2+1, 10+9+7+6+4+3+1, 10+9+7+6+5+1, 10+9+7+6+5+2+1, 10+9+7+6+5+3+1, 11+7+1, 11+7+2+1, 11+7+3+1, 11+7+4+1, 11+7+4+2+1, 11+7+4+3+1, 11+7+5+1, 11+7+5+2+1, 11+7+5+3+1, 11+7+6+1, 11+7+6+2+1, 11+7+6+4+1, 11+7+6+4+2+1, 11+7+6+4+3+1, 11+7+6+5+1, 11+7+6+5+2+1, 11+7+6+5+3+1, 11+8+7+1, 11+8+7+2+1, 11+8+7+3+1, 11+8+7+4+1, 11+8+7+4+2+1, 11+8+7+4+3+1, 11+8+7+5+1, 11+8+7+5+2+1, 11+8+7+5+3+1, 11+8+7+6+1, 11+8+7+6+2+1, 11+8+7+6+4+1, 11+8+7+6+4+2+1, 11+8+7+6+4+3+1, 11+8+7+6+5+1, 11+8+7+6+5+2+1, 11+8+7+6+5+3+1, 11+9+7+1, 11+9+7+2+1, 11+9+7+3+1, 11+9+7+4+1, 11+9+7+4+2+1, 11+9+7+4+3+1, 11+9+7+5+1, 11+9+7+5+2+1, 11+9+7+5+3+1, 11+9+7+6+1, 11+9+7+6+2+1, 11+9+7+6+4+1, 11+9+7+6+4+2+1, 11+9+7+6+4+3+1, 11+9+7+6+5+1, 11+9+7+6+5+2+1, 11+9+7+6+5+3+1, 12+11+7+1, 12+11+7+2+1, 12+11+7+3+1, 12+11+7+4+1, 12+11+7+4+2+1, 12+11+7+4+3+1, 12+11+7+5+1, 12+11+7+5+2+1, 12+11+7+5+3+1, 12+11+7+6+1, 12+11+7+6+2+1, 12+11+7+6+4+1, 12+11+7+6+4+2+1, 12+11+7+6+4+3+1, 12+11+7+6+5+1, 12+11+7+6+5+2+1, 12+11+7+6+5+3+1, 12+11+8+7+1, 12+11+8+7+2+1, 12+11+8+7+3+1, 12+11+8+7+4+1, 12+11+8+7+4+2+1, 12+11+8+7+4+3+1, 12+11+8+7+5+1, 12+11+8+7+5+2+1, 12+11+8+7+5+3+1, 12+11+8+7+6+1, 12+11+8+7+6+2+1, 12+11+8+7+6+4+1, 12+11+8+7+6+4+2+1, 12+11+8+7+6+4+3+1, 12+11+8+7+6+5+1, 12+11+8+7+6+5+2+1, 12+11+8+7+6+5+3+1, 12+11+9+7+1, 12+11+9+7+2+1, 12+11+9+7+3+1, 12+11+9+7+4+1, 12+11+9+7+4+2+1, 12+11+9+7+4+3+1, 12+11+9+7+5+1, 12+11+9+7+5+2+1, 12+11+9+7+5+3+1, 12+11+9+7+6+1, 12+11+9+7+6+2+1, 12+11+9+7+6+4+1, 12+11+9+7+6+4+2+1, 12+11+9+7+6+4+3+1, 12+11+9+7+6+5+1, 12+11+9+7+6+5+2+1, 12+11+9+7+6+5+3+1, 13+7+1, 13+7+2+1, 13+7+3+1, 13+7+4+1, 13+7+4+2+1, 13+7+4+3+1, 13+7+5+1, 13+7+5+2+1, 13+7+5+3+1, 13+7+6+1, 13+7+6+2+1, 13+7+6+4+1, 13+7+6+4+2+1, 13+7+6+4+3+1, 13+7+6+5+1, 13+7+6+5+2+1, 13+7+6+5+3+1, 13+8+7+1, 13+8+7+2+1, 13+8+7+3+1, 13+8+7+4+1, 13+8+7+4+2+1, 13+8+7+4+3+1, 13+8+7+5+1, 13+8+7+5+2+1, 13+8+7+5+3+1, 13+8+7+6+1, 13+8+7+6+2+1, 13+8+7+6+4+1, 13+8+7+6+4+2+1, 13+8+7+6+4+3+1, 13+8+7+6+5+1, 13+8+7+6+5+2+1, 13+8+7+6+5+3+1, 13+9+7+1, 13+9+7+2+1, 13+9+7+3+1, 13+9+7+4+1, 13+9+7+4+2+1, 13+9+7+4+3+1, 13+9+7+5+1, 13+9+7+5+2+1, 13+9+7+5+3+1, 13+9+7+6+1, 13+9+7+6+2+1, 13+9+7+6+4+1, 13+9+7+6+4+2+1, 13+9+7+6+4+3+1, 13+9+7+6+5+1, 13+9+7+6+5+2+1, 13+9+7+6+5+3+1, 13+10+1, 13+10+2+1, 13+10+7+1, 13+10+7+2+1, 13+10+7+3+1, 13+10+7+4+1, 13+10+7+4+2+1, 13+10+7+4+3+1, 13+10+7+5+1, 13+10+7+5+2+1, 13+10+7+5+3+1, 13+10+7+6+1, 13+10+7+6+2+1, 13+10+7+6+4+1, 13+10+7+6+4+2+1, 13+10+7+6+4+3+1, 13+10+7+6+5+1, 13+10+7+6+5+2+1, 13+10+7+6+5+3+1, 13+10+8+7+1, 13+10+8+7+2+1, 13+10+8+7+3+1, 13+10+8+7+4+1, 13+10+8+7+4+2+1, 13+10+8+7+4+3+1, 13+10+8+7+5+1, 13+10+8+7+5+2+1, 13+10+8+7+5+3+1, 13+10+8+7+6+1, 13+10+8+7+6+2+1, 13+10+8+7+6+4+1, 13+10+8+7+6+4+2+1, 13+10+8+7+6+4+3+1, 13+10+8+7+6+5+1, 13+10+8+7+6+5+2+1, 13+10+8+7+6+5+3+1, 13+10+9+7+1, 13+10+9+7+2+1, 13+10+9+7+3+1, 13+10+9+7+4+1, 13+10+9+7+4+2+1, 13+10+9+7+4+3+1, 13+10+9+7+5+1, 13+10+9+7+5+2+1, 13+10+9+7+5+3+1, 13+10+9+7+6+1, 13+10+9+7+6+2+1, 13+10+9+7+6+4+1, 13+10+9+7+6+4+2+1, 13+10+9+7+6+4+3+1, 13+10+9+7+6+5+1, 13+10+9+7+6+5+2+1, 13+10+9+7+6+5+3+1, 13+11+7+1, 13+11+7+2+1, 13+11+7+3+1, 13+11+7+4+1, 13+11+7+4+2+1, 13+11+7+4+3+1, 13+11+7+5+1, 13+11+7+5+2+1, 13+11+7+5+3+1, 13+11+7+6+1, 13+11+7+6+2+1, 13+11+7+6+4+1, 13+11+7+6+4+2+1, 13+11+7+6+4+3+1, 13+11+7+6+5+1, 13+11+7+6+5+2+1, 13+11+7+6+5+3+1, 13+11+8+7+1, 13+11+8+7+2+1, 13+11+8+7+3+1, 13+11+8+7+4+1, 13+11+8+7+4+2+1, 13+11+8+7+4+3+1, 13+11+8+7+5+1, 13+11+8+7+5+2+1, 13+11+8+7+5+3+1, 13+11+8+7+6+1, 13+11+8+7+6+2+1, 13+11+8+7+6+4+1, 13+11+8+7+6+4+2+1, 13+11+8+7+6+4+3+1, 13+11+8+7+6+5+1, 13+11+8+7+6+5+2+1, 13+11+8+7+6+5+3+1, 13+11+9+7+1, 13+11+9+7+2+1, 13+11+9+7+3+1, 13+11+9+7+4+1, 13+11+9+7+4+2+1, 13+11+9+7+4+3+1, 13+11+9+7+5+1, 13+11+9+7+5+2+1, 13+11+9+7+5+3+1, 13+11+9+7+6+1, 13+11+9+7+6+2+1, 13+11+9+7+6+4+1, 13+11+9+7+6+4+2+1, 13+11+9+7+6+4+3+1, 13+11+9+7+6+5+1, 13+11+9+7+6+5+2+1, 13+11+9+7+6+5+3+1, 13+12+11+7+1, 13+12+11+7+2+1, 13+12+11+7+3+1, 13+12+11+7+4+1, 13+12+11+7+4+2+1, 13+12+11+7+4+3+1, 13+12+11+7+5+1, 13+12+11+7+5+2+1, 13+12+11+7+5+3+1, 13+12+11+7+6+1, 13+12+11+7+6+2+1, 13+12+11+7+6+4+1, 13+12+11+7+6+4+2+1, 13+12+11+7+6+4+3+1, 13+12+11+7+6+5+1, 13+12+11+7+6+5+2+1, 13+12+11+7+6+5+3+1, 13+12+11+8+7+1, 13+12+11+8+7+2+1, 13+12+11+8+7+3+1, 13+12+11+8+7+4+1, 13+12+11+8+7+4+2+1, 13+12+11+8+7+4+3+1, 13+12+11+8+7+5+1, 13+12+11+8+7+5+2+1, 13+12+11+8+7+5+3+1, 13+12+11+8+7+6+1, 13+12+11+8+7+6+2+1, 13+12+11+8+7+6+4+1, 13+12+11+8+7+6+4+2+1, 13+12+11+8+7+6+4+3+1, 13+12+11+8+7+6+5+1, 13+12+11+8+7+6+5+2+1, 13+12+11+8+7+6+5+3+1, 13+12+11+9+7+1, 13+12+11+9+7+2+1, 13+12+11+9+7+3+1, 13+12+11+9+7+4+1, 13+12+11+9+7+4+2+1, 13+12+11+9+7+4+3+1, 13+12+11+9+7+5+1, 13+12+11+9+7+5+2+1, 13+12+11+9+7+5+3+1, 13+12+11+9+7+6+1, 13+12+11+9+7+6+2+1, 13+12+11+9+7+6+4+1, 13+12+11+9+7+6+4+2+1, 13+12+11+9+7+6+4+3+1, 13+12+11+9+7+6+5+1, 13+12+11+9+7+6+5+2+1, 13+12+11+9+7+6+5+3+1, 14+7+1, 14+7+2+1, 14+7+3+1, 14+7+4+1, 14+7+4+2+1, 14+7+4+3+1, 14+7+5+1, 14+7+5+2+1, 14+7+5+3+1, 14+7+6+1, 14+7+6+2+1, 14+7+6+4+1, 14+7+6+4+2+1, 14+7+6+4+3+1, 14+7+6+5+1, 14+7+6+5+2+1, 14+7+6+5+3+1, 14+8+7+1, 14+8+7+2+1, 14+8+7+3+1, 14+8+7+4+1, 14+8+7+4+2+1, 14+8+7+4+3+1, 14+8+7+5+1, 14+8+7+5+2+1, 14+8+7+5+3+1, 14+8+7+6+1, 14+8+7+6+2+1, 14+8+7+6+4+1, 14+8+7+6+4+2+1, 14+8+7+6+4+3+1, 14+8+7+6+5+1, 14+8+7+6+5+2+1, 14+8+7+6+5+3+1, 14+9+7+1, 14+9+7+2+1, 14+9+7+3+1, 14+9+7+4+1, 14+9+7+4+2+1, 14+9+7+4+3+1, 14+9+7+5+1, 14+9+7+5+2+1, 14+9+7+5+3+1, 14+9+7+6+1, 14+9+7+6+2+1, 14+9+7+6+4+1, 14+9+7+6+4+2+1, 14+9+7+6+4+3+1, 14+9+7+6+5+1, 14+9+7+6+5+2+1, 14+9+7+6+5+3+1, 14+10+1, 14+10+2+1, 14+10+7+1, 14+10+7+2+1, 14+10+7+3+1, 14+10+7+4+1, 14+10+7+4+2+1, 14+10+7+4+3+1, 14+10+7+5+1, 14+10+7+5+2+1, 14+10+7+5+3+1, 14+10+7+6+1, 14+10+7+6+2+1, 14+10+7+6+4+1, 14+10+7+6+4+2+1, 14+10+7+6+4+3+1, 14+10+7+6+5+1, 14+10+7+6+5+2+1, 14+10+7+6+5+3+1, 14+10+8+7+1, 14+10+8+7+2+1, 14+10+8+7+3+1, 14+10+8+7+4+1, 14+10+8+7+4+2+1, 14+10+8+7+4+3+1, 14+10+8+7+5+1, 14+10+8+7+5+2+1, 14+10+8+7+5+3+1, 14+10+8+7+6+1, 14+10+8+7+6+2+1, 14+10+8+7+6+4+1, 14+10+8+7+6+4+2+1, 14+10+8+7+6+4+3+1, 14+10+8+7+6+5+1, 14+10+8+7+6+5+2+1, 14+10+8+7+6+5+3+1, 14+10+9+7+1, 14+10+9+7+2+1, 14+10+9+7+3+1, 14+10+9+7+4+1, 14+10+9+7+4+2+1, 14+10+9+7+4+3+1, 14+10+9+7+5+1, 14+10+9+7+5+2+1, 14+10+9+7+5+3+1, 14+10+9+7+6+1, 14+10+9+7+6+2+1, 14+10+9+7+6+4+1, 14+10+9+7+6+4+2+1, 14+10+9+7+6+4+3+1, 14+10+9+7+6+5+1, 14+10+9+7+6+5+2+1, 14+10+9+7+6+5+3+1, 14+11+7+1, 14+11+7+2+1, 14+11+7+3+1, 14+11+7+4+1, 14+11+7+4+2+1, 14+11+7+4+3+1, 14+11+7+5+1, 14+11+7+5+2+1, 14+11+7+5+3+1, 14+11+7+6+1, 14+11+7+6+2+1, 14+11+7+6+4+1, 14+11+7+6+4+2+1, 14+11+7+6+4+3+1, 14+11+7+6+5+1, 14+11+7+6+5+2+1, 14+11+7+6+5+3+1, 14+11+8+7+1, 14+11+8+7+2+1, 14+11+8+7+3+1, 14+11+8+7+4+1, 14+11+8+7+4+2+1, 14+11+8+7+4+3+1, 14+11+8+7+5+1, 14+11+8+7+5+2+1, 14+11+8+7+5+3+1, 14+11+8+7+6+1, 14+11+8+7+6+2+1, 14+11+8+7+6+4+1, 14+11+8+7+6+4+2+1, 14+11+8+7+6+4+3+1, 14+11+8+7+6+5+1, 14+11+8+7+6+5+2+1, 14+11+8+7+6+5+3+1, 14+11+9+7+1, 14+11+9+7+2+1, 14+11+9+7+3+1, 14+11+9+7+4+1, 14+11+9+7+4+2+1, 14+11+9+7+4+3+1, 14+11+9+7+5+1, 14+11+9+7+5+2+1, 14+11+9+7+5+3+1, 14+11+9+7+6+1, 14+11+9+7+6+2+1, 14+11+9+7+6+4+1, 14+11+9+7+6+4+2+1, 14+11+9+7+6+4+3+1, 14+11+9+7+6+5+1, 14+11+9+7+6+5+2+1, 14+11+9+7+6+5+3+1, 14+12+11+7+1, 14+12+11+7+2+1, 14+12+11+7+3+1, 14+12+11+7+4+1, 14+12+11+7+4+2+1, 14+12+11+7+4+3+1, 14+12+11+7+5+1, 14+12+11+7+5+2+1, 14+12+11+7+5+3+1, 14+12+11+7+6+1, 14+12+11+7+6+2+1, 14+12+11+7+6+4+1, 14+12+11+7+6+4+2+1, 14+12+11+7+6+4+3+1, 14+12+11+7+6+5+1, 14+12+11+7+6+5+2+1, 14+12+11+7+6+5+3+1, 14+12+11+8+7+1, 14+12+11+8+7+2+1, 14+12+11+8+7+3+1, 14+12+11+8+7+4+1, 14+12+11+8+7+4+2+1, 14+12+11+8+7+4+3+1, 14+12+11+8+7+5+1, 14+12+11+8+7+5+2+1, 14+12+11+8+7+5+3+1, 14+12+11+8+7+6+1, 14+12+11+8+7+6+2+1, 14+12+11+8+7+6+4+1, 14+12+11+8+7+6+4+2+1, 14+12+11+8+7+6+4+3+1, 14+12+11+8+7+6+5+1, 14+12+11+8+7+6+5+2+1, 14+12+11+8+7+6+5+3+1, 14+12+11+9+7+1, 14+12+11+9+7+2+1, 14+12+11+9+7+3+1, 14+12+11+9+7+4+1, 14+12+11+9+7+4+2+1, 14+12+11+9+7+4+3+1, 14+12+11+9+7+5+1, 14+12+11+9+7+5+2+1, 14+12+11+9+7+5+3+1, 14+12+11+9+7+6+1, 14+12+11+9+7+6+2+1, 14+12+11+9+7+6+4+1, 14+12+11+9+7+6+4+2+1, 14+12+11+9+7+6+4+3+1, 14+12+11+9+7+6+5+1, 14+12+11+9+7+6+5+2+1, 14+12+11+9+7+6+5+3+1, 14+13+7+1, 14+13+7+2+1, 14+13+7+3+1, 14+13+7+4+1, 14+13+7+4+2+1, 14+13+7+4+3+1, 14+13+7+5+1, 14+13+7+5+2+1, 14+13+7+5+3+1, 14+13+7+6+1, 14+13+7+6+2+1, 14+13+7+6+4+1, 14+13+7+6+4+2+1, 14+13+7+6+4+3+1, 14+13+7+6+5+1, 14+13+7+6+5+2+1, 14+13+7+6+5+3+1, 14+13+8+7+1, 14+13+8+7+2+1, 14+13+8+7+3+1, 14+13+8+7+4+1, 14+13+8+7+4+2+1, 14+13+8+7+4+3+1, 14+13+8+7+5+1, 14+13+8+7+5+2+1, 14+13+8+7+5+3+1, 14+13+8+7+6+1, 14+13+8+7+6+2+1, 14+13+8+7+6+4+1, 14+13+8+7+6+4+2+1, 14+13+8+7+6+4+3+1, 14+13+8+7+6+5+1, 14+13+8+7+6+5+2+1, 14+13+8+7+6+5+3+1, 14+13+9+7+1, 14+13+9+7+2+1, 14+13+9+7+3+1, 14+13+9+7+4+1, 14+13+9+7+4+2+1, 14+13+9+7+4+3+1, 14+13+9+7+5+1, 14+13+9+7+5+2+1, 14+13+9+7+5+3+1, 14+13+9+7+6+1, 14+13+9+7+6+2+1, 14+13+9+7+6+4+1, 14+13+9+7+6+4+2+1, 14+13+9+7+6+4+3+1, 14+13+9+7+6+5+1, 14+13+9+7+6+5+2+1, 14+13+9+7+6+5+3+1, 14+13+10+1, 14+13+10+2+1, 14+13+10+7+1, 14+13+10+7+2+1, 14+13+10+7+3+1, 14+13+10+7+4+1, 14+13+10+7+4+2+1, 14+13+10+7+4+3+1, 14+13+10+7+5+1, 14+13+10+7+5+2+1, 14+13+10+7+5+3+1, 14+13+10+7+6+1, 14+13+10+7+6+2+1, 14+13+10+7+6+4+1, 14+13+10+7+6+4+2+1, 14+13+10+7+6+4+3+1, 14+13+10+7+6+5+1, 14+13+10+7+6+5+2+1, 14+13+10+7+6+5+3+1, 14+13+10+8+7+1, 14+13+10+8+7+2+1, 14+13+10+8+7+3+1, 14+13+10+8+7+4+1, 14+13+10+8+7+4+2+1, 14+13+10+8+7+4+3+1, 14+13+10+8+7+5+1, 14+13+10+8+7+5+2+1, 14+13+10+8+7+5+3+1, 14+13+10+8+7+6+1, 14+13+10+8+7+6+2+1, 14+13+10+8+7+6+4+1, 14+13+10+8+7+6+4+2+1, 14+13+10+8+7+6+4+3+1, 14+13+10+8+7+6+5+1, 14+13+10+8+7+6+5+2+1, 14+13+10+8+7+6+5+3+1, 14+13+10+9+7+1, 14+13+10+9+7+2+1, 14+13+10+9+7+3+1, 14+13+10+9+7+4+1, 14+13+10+9+7+4+2+1, 14+13+10+9+7+4+3+1, 14+13+10+9+7+5+1, 14+13+10+9+7+5+2+1, 14+13+10+9+7+5+3+1, 14+13+10+9+7+6+1, 14+13+10+9+7+6+2+1, 14+13+10+9+7+6+4+1, 14+13+10+9+7+6+4+2+1, 14+13+10+9+7+6+4+3+1, 14+13+10+9+7+6+5+1, 14+13+10+9+7+6+5+2+1, 14+13+10+9+7+6+5+3+1, 14+13+11+7+1, 14+13+11+7+2+1, 14+13+11+7+3+1, 14+13+11+7+4+1, 14+13+11+7+4+2+1, 14+13+11+7+4+3+1, 14+13+11+7+5+1, 14+13+11+7+5+2+1, 14+13+11+7+5+3+1, 14+13+11+7+6+1, 14+13+11+7+6+2+1, 14+13+11+7+6+4+1, 14+13+11+7+6+4+2+1, 14+13+11+7+6+4+3+1, 14+13+11+7+6+5+1, 14+13+11+7+6+5+2+1, 14+13+11+7+6+5+3+1, 14+13+11+8+7+1, 14+13+11+8+7+2+1, 14+13+11+8+7+3+1, 14+13+11+8+7+4+1, 14+13+11+8+7+4+2+1, 14+13+11+8+7+4+3+1, 14+13+11+8+7+5+1, 14+13+11+8+7+5+2+1, 14+13+11+8+7+5+3+1, 14+13+11+8+7+6+1, 14+13+11+8+7+6+2+1, 14+13+11+8+7+6+4+1, 14+13+11+8+7+6+4+2+1, 14+13+11+8+7+6+4+3+1, 14+13+11+8+7+6+5+1, 14+13+11+8+7+6+5+2+1, 14+13+11+8+7+6+5+3+1, 14+13+11+9+7+1, 14+13+11+9+7+2+1, 14+13+11+9+7+3+1, 14+13+11+9+7+4+1, 14+13+11+9+7+4+2+1, 14+13+11+9+7+4+3+1, 14+13+11+9+7+5+1, 14+13+11+9+7+5+2+1, 14+13+11+9+7+5+3+1, 14+13+11+9+7+6+1, 14+13+11+9+7+6+2+1, 14+13+11+9+7+6+4+1, 14+13+11+9+7+6+4+2+1, 14+13+11+9+7+6+4+3+1, 14+13+11+9+7+6+5+1, 14+13+11+9+7+6+5+2+1, 14+13+11+9+7+6+5+3+1, 14+13+12+11+7+1, 14+13+12+11+7+2+1, 14+13+12+11+7+3+1, 14+13+12+11+7+4+1, 14+13+12+11+7+4+2+1, 14+13+12+11+7+4+3+1, 14+13+12+11+7+5+1, 14+13+12+11+7+5+2+1, 14+13+12+11+7+5+3+1, 14+13+12+11+7+6+1, 14+13+12+11+7+6+2+1, 14+13+12+11+7+6+4+1, 14+13+12+11+7+6+4+2+1, 14+13+12+11+7+6+4+3+1, 14+13+12+11+7+6+5+1, 14+13+12+11+7+6+5+2+1, 14+13+12+11+7+6+5+3+1, 14+13+12+11+8+7+1, 14+13+12+11+8+7+2+1, 14+13+12+11+8+7+3+1, 14+13+12+11+8+7+4+1, 14+13+12+11+8+7+4+2+1, 14+13+12+11+8+7+4+3+1, 14+13+12+11+8+7+5+1, 14+13+12+11+8+7+5+2+1, 14+13+12+11+8+7+5+3+1, 14+13+12+11+8+7+6+1, 14+13+12+11+8+7+6+2+1, 14+13+12+11+8+7+6+4+1, 14+13+12+11+8+7+6+4+2+1, 14+13+12+11+8+7+6+4+3+1, 14+13+12+11+8+7+6+5+1, 14+13+12+11+8+7+6+5+2+1, 14+13+12+11+8+7+6+5+3+1, 14+13+12+11+9+7+1, 14+13+12+11+9+7+2+1, 14+13+12+11+9+7+3+1, 14+13+12+11+9+7+4+1, 14+13+12+11+9+7+4+2+1, 14+13+12+11+9+7+4+3+1, 14+13+12+11+9+7+5+1, 14+13+12+11+9+7+5+2+1, 14+13+12+11+9+7+5+3+1, 14+13+12+11+9+7+6+1, 14+13+12+11+9+7+6+2+1, 14+13+12+11+9+7+6+4+1, 14+13+12+11+9+7+6+4+2+1, 14+13+12+11+9+7+6+4+3+1, 14+13+12+11+9+7+6+5+1, 14+13+12+11+9+7+6+5+2+1, 14+13+12+11+9+7+6+5+3+1, 15+7+1, 15+7+2+1, 15+7+3+1, 15+7+4+1, 15+7+4+2+1, 15+7+4+3+1, 15+7+5+1, 15+7+5+2+1, 15+7+5+3+1, 15+7+6+1, 15+7+6+2+1, 15+7+6+4+1, 15+7+6+4+2+1, 15+7+6+4+3+1, 15+7+6+5+1, 15+7+6+5+2+1, 15+7+6+5+3+1, 15+8+7+1, 15+8+7+2+1, 15+8+7+3+1, 15+8+7+4+1, 15+8+7+4+2+1, 15+8+7+4+3+1, 15+8+7+5+1, 15+8+7+5+2+1, 15+8+7+5+3+1, 15+8+7+6+1, 15+8+7+6+2+1, 15+8+7+6+4+1, 15+8+7+6+4+2+1, 15+8+7+6+4+3+1, 15+8+7+6+5+1, 15+8+7+6+5+2+1, 15+8+7+6+5+3+1, 15+9+7+1, 15+9+7+2+1, 15+9+7+3+1, 15+9+7+4+1, 15+9+7+4+2+1, 15+9+7+4+3+1, 15+9+7+5+1, 15+9+7+5+2+1, 15+9+7+5+3+1, 15+9+7+6+1, 15+9+7+6+2+1, 15+9+7+6+4+1, 15+9+7+6+4+2+1, 15+9+7+6+4+3+1, 15+9+7+6+5+1, 15+9+7+6+5+2+1, 15+9+7+6+5+3+1, 15+10+1, 15+10+2+1, 15+10+7+1, 15+10+7+2+1, 15+10+7+3+1, 15+10+7+4+1, 15+10+7+4+2+1, 15+10+7+4+3+1, 15+10+7+5+1, 15+10+7+5+2+1, 15+10+7+5+3+1, 15+10+7+6+1, 15+10+7+6+2+1, 15+10+7+6+4+1, 15+10+7+6+4+2+1, 15+10+7+6+4+3+1, 15+10+7+6+5+1, 15+10+7+6+5+2+1, 15+10+7+6+5+3+1, 15+10+8+7+1, 15+10+8+7+2+1, 15+10+8+7+3+1, 15+10+8+7+4+1, 15+10+8+7+4+2+1, 15+10+8+7+4+3+1, 15+10+8+7+5+1, 15+10+8+7+5+2+1, 15+10+8+7+5+3+1, 15+10+8+7+6+1, 15+10+8+7+6+2+1, 15+10+8+7+6+4+1, 15+10+8+7+6+4+2+1, 15+10+8+7+6+4+3+1, 15+10+8+7+6+5+1, 15+10+8+7+6+5+2+1, 15+10+8+7+6+5+3+1, 15+10+9+7+1, 15+10+9+7+2+1, 15+10+9+7+3+1, 15+10+9+7+4+1, 15+10+9+7+4+2+1, 15+10+9+7+4+3+1, 15+10+9+7+5+1, 15+10+9+7+5+2+1, 15+10+9+7+5+3+1, 15+10+9+7+6+1, 15+10+9+7+6+2+1, 15+10+9+7+6+4+1, 15+10+9+7+6+4+2+1, 15+10+9+7+6+4+3+1, 15+10+9+7+6+5+1, 15+10+9+7+6+5+2+1, 15+10+9+7+6+5+3+1, 15+11+7+1, 15+11+7+2+1, 15+11+7+3+1, 15+11+7+4+1, 15+11+7+4+2+1, 15+11+7+4+3+1, 15+11+7+5+1, 15+11+7+5+2+1, 15+11+7+5+3+1, 15+11+7+6+1, 15+11+7+6+2+1, 15+11+7+6+4+1, 15+11+7+6+4+2+1, 15+11+7+6+4+3+1, 15+11+7+6+5+1, 15+11+7+6+5+2+1, 15+11+7+6+5+3+1, 15+11+8+7+1, 15+11+8+7+2+1, 15+11+8+7+3+1, 15+11+8+7+4+1, 15+11+8+7+4+2+1, 15+11+8+7+4+3+1, 15+11+8+7+5+1, 15+11+8+7+5+2+1, 15+11+8+7+5+3+1, 15+11+8+7+6+1, 15+11+8+7+6+2+1, 15+11+8+7+6+4+1, 15+11+8+7+6+4+2+1, 15+11+8+7+6+4+3+1, 15+11+8+7+6+5+1, 15+11+8+7+6+5+2+1, 15+11+8+7+6+5+3+1, 15+11+9+7+1, 15+11+9+7+2+1, 15+11+9+7+3+1, 15+11+9+7+4+1, 15+11+9+7+4+2+1, 15+11+9+7+4+3+1, 15+11+9+7+5+1, 15+11+9+7+5+2+1, 15+11+9+7+5+3+1, 15+11+9+7+6+1, 15+11+9+7+6+2+1, 15+11+9+7+6+4+1, 15+11+9+7+6+4+2+1, 15+11+9+7+6+4+3+1, 15+11+9+7+6+5+1, 15+11+9+7+6+5+2+1, 15+11+9+7+6+5+3+1, 15+12+11+7+1, 15+12+11+7+2+1, 15+12+11+7+3+1, 15+12+11+7+4+1, 15+12+11+7+4+2+1, 15+12+11+7+4+3+1, 15+12+11+7+5+1, 15+12+11+7+5+2+1, 15+12+11+7+5+3+1, 15+12+11+7+6+1, 15+12+11+7+6+2+1, 15+12+11+7+6+4+1, 15+12+11+7+6+4+2+1, 15+12+11+7+6+4+3+1, 15+12+11+7+6+5+1, 15+12+11+7+6+5+2+1, 15+12+11+7+6+5+3+1, 15+12+11+8+7+1, 15+12+11+8+7+2+1, 15+12+11+8+7+3+1, 15+12+11+8+7+4+1, 15+12+11+8+7+4+2+1, 15+12+11+8+7+4+3+1, 15+12+11+8+7+5+1, 15+12+11+8+7+5+2+1, 15+12+11+8+7+5+3+1, 15+12+11+8+7+6+1, 15+12+11+8+7+6+2+1, 15+12+11+8+7+6+4+1, 15+12+11+8+7+6+4+2+1, 15+12+11+8+7+6+4+3+1, 15+12+11+8+7+6+5+1, 15+12+11+8+7+6+5+2+1, 15+12+11+8+7+6+5+3+1, 15+12+11+9+7+1, 15+12+11+9+7+2+1, 15+12+11+9+7+3+1, 15+12+11+9+7+4+1, 15+12+11+9+7+4+2+1, 15+12+11+9+7+4+3+1, 15+12+11+9+7+5+1, 15+12+11+9+7+5+2+1, 15+12+11+9+7+5+3+1, 15+12+11+9+7+6+1, 15+12+11+9+7+6+2+1, 15+12+11+9+7+6+4+1, 15+12+11+9+7+6+4+2+1, 15+12+11+9+7+6+4+3+1, 15+12+11+9+7+6+5+1, 15+12+11+9+7+6+5+2+1, 15+12+11+9+7+6+5+3+1, 15+13+7+1, 15+13+7+2+1, 15+13+7+3+1, 15+13+7+4+1, 15+13+7+4+2+1, 15+13+7+4+3+1, 15+13+7+5+1, 15+13+7+5+2+1, 15+13+7+5+3+1, 15+13+7+6+1, 15+13+7+6+2+1, 15+13+7+6+4+1, 15+13+7+6+4+2+1, 15+13+7+6+4+3+1, 15+13+7+6+5+1, 15+13+7+6+5+2+1, 15+13+7+6+5+3+1, 15+13+8+7+1, 15+13+8+7+2+1, 15+13+8+7+3+1, 15+13+8+7+4+1, 15+13+8+7+4+2+1, 15+13+8+7+4+3+1, 15+13+8+7+5+1, 15+13+8+7+5+2+1, 15+13+8+7+5+3+1, 15+13+8+7+6+1, 15+13+8+7+6+2+1, 15+13+8+7+6+4+1, 15+13+8+7+6+4+2+1, 15+13+8+7+6+4+3+1, 15+13+8+7+6+5+1, 15+13+8+7+6+5+2+1, 15+13+8+7+6+5+3+1, 15+13+9+7+1, 15+13+9+7+2+1, 15+13+9+7+3+1, 15+13+9+7+4+1, 15+13+9+7+4+2+1, 15+13+9+7+4+3+1, 15+13+9+7+5+1, 15+13+9+7+5+2+1, 15+13+9+7+5+3+1, 15+13+9+7+6+1, 15+13+9+7+6+2+1, 15+13+9+7+6+4+1, 15+13+9+7+6+4+2+1, 15+13+9+7+6+4+3+1, 15+13+9+7+6+5+1, 15+13+9+7+6+5+2+1, 15+13+9+7+6+5+3+1, 15+13+10+1, 15+13+10+2+1, 15+13+10+7+1, 15+13+10+7+2+1, 15+13+10+7+3+1, 15+13+10+7+4+1, 15+13+10+7+4+2+1, 15+13+10+7+4+3+1, 15+13+10+7+5+1, 15+13+10+7+5+2+1, 15+13+10+7+5+3+1, 15+13+10+7+6+1, 15+13+10+7+6+2+1, 15+13+10+7+6+4+1, 15+13+10+7+6+4+2+1, 15+13+10+7+6+4+3+1, 15+13+10+7+6+5+1, 15+13+10+7+6+5+2+1, 15+13+10+7+6+5+3+1, 15+13+10+8+7+1, 15+13+10+8+7+2+1, 15+13+10+8+7+3+1, 15+13+10+8+7+4+1, 15+13+10+8+7+4+2+1, 15+13+10+8+7+4+3+1, 15+13+10+8+7+5+1, 15+13+10+8+7+5+2+1, 15+13+10+8+7+5+3+1, 15+13+10+8+7+6+1, 15+13+10+8+7+6+2+1, 15+13+10+8+7+6+4+1, 15+13+10+8+7+6+4+2+1, 15+13+10+8+7+6+4+3+1, 15+13+10+8+7+6+5+1, 15+13+10+8+7+6+5+2+1, 15+13+10+8+7+6+5+3+1, 15+13+10+9+7+1, 15+13+10+9+7+2+1, 15+13+10+9+7+3+1, 15+13+10+9+7+4+1, 15+13+10+9+7+4+2+1, 15+13+10+9+7+4+3+1, 15+13+10+9+7+5+1, 15+13+10+9+7+5+2+1, 15+13+10+9+7+5+3+1, 15+13+10+9+7+6+1, 15+13+10+9+7+6+2+1, 15+13+10+9+7+6+4+1, 15+13+10+9+7+6+4+2+1, 15+13+10+9+7+6+4+3+1, 15+13+10+9+7+6+5+1, 15+13+10+9+7+6+5+2+1, 15+13+10+9+7+6+5+3+1, 15+13+11+7+1, 15+13+11+7+2+1, 15+13+11+7+3+1, 15+13+11+7+4+1, 15+13+11+7+4+2+1, 15+13+11+7+4+3+1, 15+13+11+7+5+1, 15+13+11+7+5+2+1, 15+13+11+7+5+3+1, 15+13+11+7+6+1, 15+13+11+7+6+2+1, 15+13+11+7+6+4+1, 15+13+11+7+6+4+2+1, 15+13+11+7+6+4+3+1, 15+13+11+7+6+5+1, 15+13+11+7+6+5+2+1, 15+13+11+7+6+5+3+1, 15+13+11+8+7+1, 15+13+11+8+7+2+1, 15+13+11+8+7+3+1, 15+13+11+8+7+4+1, 15+13+11+8+7+4+2+1, 15+13+11+8+7+4+3+1, 15+13+11+8+7+5+1, 15+13+11+8+7+5+2+1, 15+13+11+8+7+5+3+1, 15+13+11+8+7+6+1, 15+13+11+8+7+6+2+1, 15+13+11+8+7+6+4+1, 15+13+11+8+7+6+4+2+1, 15+13+11+8+7+6+4+3+1, 15+13+11+8+7+6+5+1, 15+13+11+8+7+6+5+2+1, 15+13+11+8+7+6+5+3+1, 15+13+11+9+7+1, 15+13+11+9+7+2+1, 15+13+11+9+7+3+1, 15+13+11+9+7+4+1, 15+13+11+9+7+4+2+1, 15+13+11+9+7+4+3+1, 15+13+11+9+7+5+1, 15+13+11+9+7+5+2+1, 15+13+11+9+7+5+3+1, 15+13+11+9+7+6+1, 15+13+11+9+7+6+2+1, 15+13+11+9+7+6+4+1, 15+13+11+9+7+6+4+2+1, 15+13+11+9+7+6+4+3+1, 15+13+11+9+7+6+5+1, 15+13+11+9+7+6+5+2+1, 15+13+11+9+7+6+5+3+1, 15+13+12+11+7+1, 15+13+12+11+7+2+1, 15+13+12+11+7+3+1, 15+13+12+11+7+4+1, 15+13+12+11+7+4+2+1, 15+13+12+11+7+4+3+1, 15+13+12+11+7+5+1, 15+13+12+11+7+5+2+1, 15+13+12+11+7+5+3+1, 15+13+12+11+7+6+1, 15+13+12+11+7+6+2+1, 15+13+12+11+7+6+4+1, 15+13+12+11+7+6+4+2+1, 15+13+12+11+7+6+4+3+1, 15+13+12+11+7+6+5+1, 15+13+12+11+7+6+5+2+1, 15+13+12+11+7+6+5+3+1, 15+13+12+11+8+7+1, 15+13+12+11+8+7+2+1, 15+13+12+11+8+7+3+1, 15+13+12+11+8+7+4+1, 15+13+12+11+8+7+4+2+1, 15+13+12+11+8+7+4+3+1, 15+13+12+11+8+7+5+1, 15+13+12+11+8+7+5+2+1, 15+13+12+11+8+7+5+3+1, 15+13+12+11+8+7+6+1, 15+13+12+11+8+7+6+2+1, 15+13+12+11+8+7+6+4+1, 15+13+12+11+8+7+6+4+2+1, 15+13+12+11+8+7+6+4+3+1, 15+13+12+11+8+7+6+5+1, 15+13+12+11+8+7+6+5+2+1, 15+13+12+11+8+7+6+5+3+1, 15+13+12+11+9+7+1, 15+13+12+11+9+7+2+1, 15+13+12+11+9+7+3+1, 15+13+12+11+9+7+4+1, 15+13+12+11+9+7+4+2+1, 15+13+12+11+9+7+4+3+1, 15+13+12+11+9+7+5+1, 15+13+12+11+9+7+5+2+1, 15+13+12+11+9+7+5+3+1, 15+13+12+11+9+7+6+1, 15+13+12+11+9+7+6+2+1, 15+13+12+11+9+7+6+4+1, 15+13+12+11+9+7+6+4+2+1, 15+13+12+11+9+7+6+4+3+1, 15+13+12+11+9+7+6+5+1, 15+13+12+11+9+7+6+5+2+1, 15+13+12+11+9+7+6+5+3+1, 16+1, 16+2+1, 16+3+1, 16+4+1, 16+4+2+1, 16+4+3+1, 16+5+1, 16+5+2+1, 16+5+3+1, 16+6+1, 16+6+2+1, 16+6+4+1, 16+6+4+2+1, 16+6+4+3+1, 16+6+5+1, 16+6+5+2+1, 16+6+5+3+1, 17+16+1, 17+16+2+1, 17+16+3+1, 17+16+4+1, 17+16+4+2+1, 17+16+4+3+1, 17+16+5+1, 17+16+5+2+1, 17+16+5+3+1, 17+16+6+1, 17+16+6+2+1, 17+16+6+4+1, 17+16+6+4+2+1, 17+16+6+4+3+1, 17+16+6+5+1, 17+16+6+5+2+1, 17+16+6+5+3+1, 18+16+1, 18+16+2+1, 18+16+3+1, 18+16+4+1, 18+16+4+2+1, 18+16+4+3+1, 18+16+5+1, 18+16+5+2+1, 18+16+5+3+1, 18+16+6+1, 18+16+6+2+1, 18+16+6+4+1, 18+16+6+4+2+1, 18+16+6+4+3+1, 18+16+6+5+1, 18+16+6+5+2+1, 18+16+6+5+3+1, 19+16+1, 19+16+2+1, 19+16+3+1, 19+16+4+1, 19+16+4+2+1, 19+16+4+3+1, 19+16+5+1, 19+16+5+2+1, 19+16+5+3+1, 19+16+6+1, 19+16+6+2+1, 19+16+6+4+1, 19+16+6+4+2+1, 19+16+6+4+3+1, 19+16+6+5+1, 19+16+6+5+2+1, 19+16+6+5+3+1, 20+19+16+1, 20+19+16+2+1, 20+19+16+3+1, 20+19+16+4+1, 20+19+16+4+2+1, 20+19+16+4+3+1, 20+19+16+5+1, 20+19+16+5+2+1, 20+19+16+5+3+1, 20+19+16+6+1, 20+19+16+6+2+1, 20+19+16+6+4+1, 20+19+16+6+4+2+1, 20+19+16+6+4+3+1, 20+19+16+6+5+1, 20+19+16+6+5+2+1, 20+19+16+6+5+3+1, 21+1, 21+2+1, 22+1, 22+2+1, 23+1, 23+2+1, 24+23+1, 24+23+2+1, 25+23+1, 25+23+2+1, 26, 27+1, 27+2+1, 28+1, 28+2+1, 29+1 and 30.

In the list above, the numbers refer to the embodiments according to their numbering provided hereinabove whereas “+” indicates the dependency from another embodiment. The different individualised embodiments are separated by commas. In other words, “4+3+1” for example refers to embodiment 4) depending on embodiment 3), depending on embodiment 1), i.e. embodiment “4+3+1” corresponds to embodiment 1) further limited by the features of embodiments 3) and 4).

It is understood that compounds of formula I wherein R¹ is not H and/or wherein the group R^{1B} comprises a 2-dimethylaminoacetoxy, phosphonooxy, [(2-(phosphonooxy-(C₁-C₄)alkyl)-phenyl)-(C₁-C₄)alkyl]carbonyloxy, [2-(phosphonooxy-(C₁-C₄)alkyl)-phenyl]carbonyloxy, or [(2-phosphonooxy-phenyl)-(C₁-C₄)alkyl]carbonyloxy group may require bioactivation by phosphatases and/or esterases and/or any biological system to exert their antibacterial activity upon administration to humans.

The compounds of formula I can be manufactured in accordance with the present invention using the procedures described hereafter.

Preparation of the Compounds of Formula I

Abbreviations:

The following abbreviations are used throughout the specification and the examples:
Ac acetyl
AcOH acetic acid
AIBN azobisisobutyronitrile
aq. aqueous
Boc tert-butoxycarbonyl
CC column chromatography over silica gel
Cipro ciprofloxacin
Cy cyclohexyl
DAD diode array detection
dba dibenzylideneacetone
DCC dicyclohexylcarbodiimide
DCE 1,2-dichloroethane
DCM dichloromethane
DEA diethylamine
DIPEA N,N-diisopropylethylamine
DMAP 4-dimethylamino-pyridine
DME 1,2-dimethoxyethane
DMF N,N-dimethylformamide
DMSO dimethylsulfoxide
DSC N,N′-disuccinimidyl carbonate
EA ethyl acetate
EDC N-(3-dimethylaminopropyl)-N′-ethylcarbodiimide hydrochloride
e.e. enantiomeric excess
ELSD evaporative light scattering detector
ESI electron spray ionisation
eq. equivalent(s)
Et ethyl
Et₂O diethyl ether
EtOH ethanol
h hour(s)
HATU O-(7-azabenzotriazol-1-yl)-N,N,N′,N′-tetramethyluronium hexafluorophosphate
Hept heptane
Hex hexane
HOBT hydroxybenzotriazole
HPLC high performance liquid chromatography
iPr isopropyl
iPrOH isopropanol
IT internal temperature
LC-MS liquid chromatography-mass spectroscopy
LDA lithium diisopropyl amide
LiHMDS lithium hexamethyldisilazide
MCPBA 3-chloro perbenzoic acid
Me methyl
MeCN acetonitrile
MeOH methanol
min minute(s)
MS mass spectroscopy
Ms methanesulfonyl (mesyl)
NBS N-bromosuccinimide
n-Bu n-butyl
NMP N-methyl-2-pyrrolidone
NMR Nuclear Magnetic Resonance
org. organic
Pd/C palladium on carbon
PEPPSI™-IPr [1,3-bis(2,6-diisopropylphenyl)imidazol-2-ylidene](3-chloropyridyl)palladium(II) dichloride
Ph phenyl
PPTS para-toluenesulfonic acid pyridinium salt
prep-HPLC preparative HPLC
Pyr pyridine
Q-phos 1,2,3,4,5-pentaphenyl-1′-(di-tert-butylphosphino)ferrocene
quant. quantitative
rt room temperature
sat. saturated
SK-CC01-A 2′-(dimethylamino)-2-biphenylyl-palladium(II) chloride dinorbomylphosphine complex
S-Phos 2-dicyclohexylphosphino-2′,6′-dimethoxybiphenyl
TBAF tetra-n-butylammonium fluoride
TBDPSCl tert-butyldiphenylsilyl chloride
TBME tert-butylmethyl ether
tBu tert-butyl
TEA triethylamine
TFA trifluoroacetic acid
THF tetrahydrofuran
THP tetrahydropyranyl
TLC thin layer chromatography
TMS trimethylsilyl
TMSE 2-(trimethylsilyl)ethyl
tR retention time
Ts para-toluenesulfonyl
UV ultra violet

General Reaction Techniques:

General Reaction Technique 1 (Hydroxamic Acid Protecting Group or Phosphonic Acid Protecting Group Removal);

The protecting groups of hydroxamic acid ester derivatives (CONHOPG) or the protecting groups of phosphonic acid ester derivatives (P(O)(OPG′)₂) are removed as follows:
When PG or PG′ is THP, (2-methylpropoxy)ethyl, methoxymethyl, tBu, COOtBu or COtBu: by acidic treatment with e.g. TFA or HCl in an org. solvent such as DCM, dioxane, Et₂O or MeOH between 0° C. and rt or by treatment with pyridinium para-toluenesulfonate in EtOH between rt and +80° C.;
When PG or PG′ is trityl: by treatment with diluted acid such as citric acid or HCl in an org. solvent such as MeOH or DCM;
When PG or PG′ is TMSE: by using fluoride anion sources such as BF₃.etherate complex in MeCN at 0° C., TBAF in THF between 0° C. and +40° C. or HF in MeCN or water between 0° C. and +40° C., or using acidic conditions such as AcOH in THF/MeOH or HCl in MeOH;
When PG or PG′ is allyl: by treatment with Pd(PPh₃)₄ in a solvent such as MeOH in presence of K₂CO₃ or a scavenger such as dimedone, morpholine or tributyltin hydride.

Further general methods to remove hydroxamic acid protecting groups have been described in T. W. Greene & P. G. M. Wuts, Protecting Groups in Organic Synthesis, 3^{rd} Ed (1999), 23-147 (Publisher: John Wiley and Sons, Inc., New York, N.Y.).

General Reaction Technique 2 (Amide Bond Formation):

The carboxylic acid is reacted with the hydroxylamine or amine derivative in the presence of an activating agent such as DCC, EDC, HOBT, n-propylphosphonic cyclic anhydride, HATU or DSC, in a dry aprotic solvent such as DCM, MeCN or DMF between −20° C. and 60° C. (see G. Benz in Comprehensive Organic Synthesis, B. M. Trost, I. Fleming, Eds; Pergamon Press: New York (1991), vol. 6, p. 381). Alternatively, the carboxylic acid can be activated by conversion into its corresponding acid chloride by reaction with oxalyl chloride or thionyl chloride neat or in a solvent like DCM between −20° and 60° C. Further activating agents can be found in R. C. Larock, Comprehensive Organic Transformations. A guide to Functional Group Preparations, 2^{nd} Edition (1999), section nitriles, carboxylic acids and derivatives, p. 1941-1949 (Wiley-VC; New York, Chichester, Weinheim, Brisbane, Singapore, Toronto).

General Reaction Technique 3 (Suzuki Cross-Coupling Reaction)

The aromatic halide (typically a bromide) is reacted with the required boronic acid derivative or its boronate ester equivalent (e.g. pinacol ester) in the presence of a palladium catalyst and a base such as K₂CO₃, Cs₂CO₃, K₃PO₄, tBuONa or tBuOK between 20 and 120° C. in a solvent such as toluene, THF, dioxane, DME or DMF, usually in the presence of water (20 to 50%). Examples of typical palladium catalysts are triarylphosphine palladium complexes such as Pd(PPh₃)₄. These catalysts can also be prepared in situ from a common palladium source such as Pd(OAc)₂ or Pd₂(dba)₃ and a ligand such as trialkylphosphines (e.g. PCy₃ or P(tBu)₃), dialkylphosphinobiphenyls (e.g. S-Phos) or ferrocenylphosphines (e.g. Q-phos). Alternatively, one can use a commercially available precatalyst based on palladacycle (e.g. SK-CC01-A) or N-heterocyclic carbene complexes (e.g. PEPPSI™-IPr). The reaction can also be performed by using the corresponding aromatic triflate. Further variations of the reaction are described in Miyaura and Suzuki, Chem. Rev. (1995), 95, 2457-2483, Bellina et al., Synthesis (2004), 2419-2440, Mauger and Mignani, Aldrichimica Acta (2006), 39, 17-24, Kantchev et al., Aldrichimica Acta (2006), 39, 97-111, Fu, Acc. Chem. Res. (2008), 41, 1555-1564, and references cited therein.

General Reaction Technique 4 (Haloaryl-Alkyne or Alkyne-Haloalkyne Cross-Coupling)

An alkyne derivative is coupled with a haloaryl or a haloalkyne derivative using a catalytic amount of a palladium salt, an org. base such as TEA and a catalytic amount of a copper derivative (usually copper iodide) in a solvent such as DMF at a temperature from 20 to 100° C. (see Sonogashira, K. in Metal-Catalyzed Reactions, Diederich, F., Stang, P. J., Eds.; Wiley-VCH: New York (1998)). Alternatively, the alkyne-haloalkyne cross coupling reaction can be performed using only a catalytic amount of copper derivative in presence of aqueous hydroxylamine and a base such as piperidine or pyrrolidine (see Chodkiewicz and Cadiot, C. R. Hebd. Seances Acad. Sci. (1955), 241, 1055-1057).

General Reaction Technique 5 (Transformation of an Ester into an Acid):

When the ester side chain is a linear alkyl, the hydrolysis is usually performed by treatment with an alkali hydroxide such as LiOH, KOH or NaOH in a water-dioxan or water-THF mixture between 0° C. and 80° C. When the ester side chain is tBu, the release of the corresponding acid can also be performed in neat TFA or diluted TFA or HCl in an org. solvent such as ether or THF. When the ester side chain is the allyl group, the reaction is performed in the presence of tetrakis(triphenylphosphine)palladium(0) in the presence of an allyl cation scavenger such as morpholine, dimedone or tributyltin hydride between 0° C. and 50° C. in a solvent such as THF. When the ester side chain is benzyl, the reaction is performed under hydrogen in the presence of a noble metal catalyst such as Pd/C in a solvent such as MeOH, THF or EA. Further strategies to introduce other acid protecting groups and general methods to remove them have been described in T. W. Greene & P. G. M. Wuts, Protecting Groups in Organic Synthesis, 3^{rd} Ed. (1999), 369-441 (Publisher: John Wiley and Sons, Inc., New York, N.Y.).

General Reaction Technique 6 (Reductive Amination):

The reaction between the amine and the aldehyde or ketone is performed in a solvent system allowing the removal of the formed water through physical or chemical means (e.g. distillation of the solvent-water azeotrope or presence of drying agents such as molecular sieves, MgSO₄ or Na₂SO₄). Such solvent is typically toluene, Hex, THF, DCM or DCE or a mixture of solvents such as DCE/MeOH. The reaction can be catalyzed by traces of acid (usually AcOH). The intermediate imine is reduced with a suitable reducing agent (e.g. NaBH₄, NaBHCN₃, or NaBH(OAc)₃ or through hydrogenation over a noble metal catalyst such as Pd/C. The reaction is carried out between −10° C. and 110° C., preferably between 0° C. and 60° C. The reaction can also be carried out in one pot. It can also be performed in protic solvents such as MeOH or water in presence of a picoline-borane complex (Tetrahedron (2004), 60, 7899-7906).

General Preparation Methods:

Preparation of the Compounds of Formula I:

The compounds of formula I can be manufactured by the methods given below, by the methods given in the examples or by analogous methods. Optimum reaction conditions may vary with the particular reactants or solvents used, but such conditions can be determined by a person skilled in the art by routine optimisation procedures.

The sections hereafter describe general methods for preparing compounds of formula I. If not indicated otherwise, the generic groups R¹, R², X, M^{A}, M^{B}, R^{1A}, R^{2A}, R^{3A} and R^{1B} are as defined for formula I. General synthetic methods used repeatedly throughout the text below are referenced to and described in the above section entitled “General reaction techniques”. In some instances certain generic groups might be incompatible with the assembly illustrated in the procedures and schemes below and so will require the use of protecting groups. The use of protecting groups is well known in the art (see for example T. W. Greene, P. G. M. Wuts, Protective Groups in Organic Synthesis, 3^{ed} Ed (1999), Wiley-Interscience).

Preparation of the Compounds of Formula I Wherein R¹=H:

The compounds of formula I wherein R¹=H can be obtained by deprotecting a compound of formula II

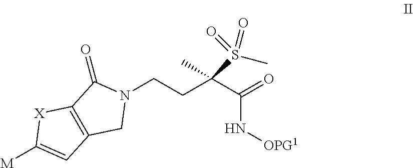

wherein X and M have the same meaning as in formula I and PG¹ represents THP, TMSE, trityl, (2-methylpropoxy)ethyl, methoxymethyl, allyl, tBu, COOtBu or COtBu using general reaction technique 1. The reaction can also be performed with racemic material and the (R) enantiomer can be obtained by chiral HPLC separation.

If desired, the compounds of formula I thus obtained may be converted into their salts, and notably into their pharmaceutically acceptable salts using standard methods.

Besides, whenever the compounds of formula I are obtained in the form of mixtures of enantiomers, the enantiomers can be separated using methods known to one skilled in the art, e.g. by formation and separation of diastereomeric salts or by HPLC over a chiral stationary phase such as a Regis Whelk-O1(R,R) (10 μm) column, a Daicel ChiralCel OD-H (5-10 μm) column, or a Daicel ChiralPak IA (10 μm) or AD-H (5 μm) column. Typical conditions of chiral HPLC are an isocratic mixture of eluent A (EtOH, in the presence or absence of an amine such as TEA or diethylamine) and eluent B (Hex), at a flow rate of 0.8 to 150 mL/min.

Preparation of the Compounds of Formula I Wherein R¹≠H:

The compounds of formula I wherein R¹≠H can be obtained by:

a) reacting a compound of formula I wherein R¹=H, and X and M are as defined in formula I with a compound of formula III

(PG^{A}O)₂P—N(iPr)₂ III

wherein PG^{A} represents tert-butyl. The reaction is performed in presence of a base such as tetrazole in a solvent such as acetonitrile at a temperature of about 0° C. An oxidation reaction is subsequently performed adding an oxidizing agent such as hydrogen peroxide in water or MCPBA (this sequence can also be performed with racemic compound of formula I wherein R=H and the (R)-enantiomer can then be obtained by chiral HPLC separation of the reaction product). Functional groups present on M that would be incompatible with the reaction conditions abovementioned can be protected before performing said reaction and deprotected after performing said reaction. Final cleavage of PG^{A} can be performed using general reaction technique 1 leading to compounds of formula I wherein R¹=PO₃H₂.

b) reacting a compound of formula I wherein R¹=H, and X and M are as defined in formula I with a compound of formula IV

HOC(O)R² IV

wherein R² has the same meaning as in formula I. The reaction can be performed using general reaction technique 2 leading to compounds of Formula I wherein R¹=C(O)R² (this reaction can also be performed with racemic compound of formula I wherein R=H and the (R)-enantiomer can then be obtained by chiral HPLC separation of the reaction product). Functional groups present on R² and M that would be incompatible with the reaction conditions abovementioned can be protected before performing said reaction and deprotected after performing said reaction.

c) reacting a compound of formula I wherein R¹=H, and X and M are as defined in formula I with a compound of formula V

Y^{a}—(CH₂)—O—P(O)(OPG^{A})₂ V

wherein Y^{a} represents iodine, bromine or chlorine and PG^{A} has the same meaning as in formula III. The reaction can be performed in presence of a mineral base such as NaH or K₂CO₃ or in presence of an organic base such as TEA or DIPEA in a solvent such as THF at a temperature ranging between about −50° C. and rt. Functional groups present on M that would be incompatible with the reaction conditions abovementioned can be protected before performing said reaction and deprotected after performing said reaction. This sequence can also be performed with racemic compound of formula I wherein R=H and the (R)-enantiomer can then be obtained by chiral HPLC separation of the reaction product. Final cleavage of PG^{A} can be performed using general reaction technique 1 leading to compounds of formula I wherein R¹=CH₂—O—PO₃H₂.

d) reacting a compound of formula I wherein R¹=H, and X and M are as defined in formula I with Pyr.SO₃ complex or Me₂NCHO.SO₃ complex in a solvent such as DMF or pyridine leading to compounds of formula I wherein R¹=SO₃H. Functional groups present on M that would be incompatible with the reaction conditions abovementioned can be protected before performing said reaction and deprotected after performing said reaction. This sequence can also be performed with racemic compound of formula I wherein R=H and the (R)-enantiomer can then be obtained by chiral HPLC separation of the reaction product.

If desired, the compounds of formula I thus obtained may be converted into their salts, and notably into their pharmaceutically acceptable salts using standard methods.

Besides, whenever the compounds of formula I are obtained in the form of mixtures of enantiomers, the enantiomers can be separated using methods known to one skilled in the art, e.g. by formation and separation of diastereomeric salts or by HPLC over a chiral stationary phase such as a Regis Whelk-O1(R,R) (10 μm) column, a Daicel ChiralCel OD-H (5-10 μm) column, or a Daicel ChiralPak IA (10 μm) or AD-H (5 μm) column. Typical conditions of chiral HPLC are an isocratic mixture of eluent A (EtOH, in the presence or absence of an amine such as TEA or diethylamine) and eluent B (Hex), at a flow rate of 0.8 to 150 mL/min.

Preparation of the Compounds of Formula II:

The compounds of formula II can be obtained by:

a) reacting a compound of formula VI

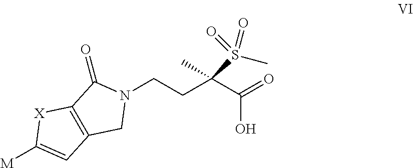

wherein X and M are as defined in formula I with a compound of formula VII

H₂N-OPG¹ VII
wherein PG¹ has the same meaning as in formula II using general reaction technique 2 (this reaction can also be performed with racemic compound of formula VI and the (R)-enantiomer can then be obtained by chiral HPLC separation of the reaction product), whereby functional groups present on M that would be incompatible with the coupling conditions mentioned in general reaction technique 2 can be protected before performing said reaction and deprotected after performing said reaction; or

b) reacting a boron derivative of formula VIII

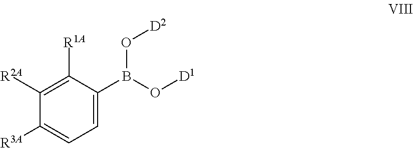

wherein R^{1A}, R^{2A} and R^{3A} have the same respective meanings as in formula I, D¹ and D² represent H, methyl or ethyl or D¹ and D² together represent CH₂C(Me)₂CH₂ or C(Me)₂C(Me)₂ with a compound of formula IX

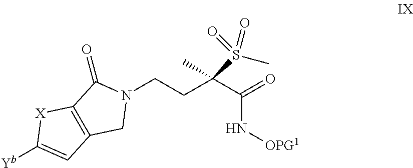

wherein X is as defined in formula I, Y^{b} represents a halogen such as bromine or iodine and PG¹ has the same meaning as in formula II, using general reaction technique 3 leading to compounds of formula II wherein M represents M^{A} and A represents a bond (this reaction can also be performed with racemic compound of formula IX and the (R)-enantiomer can then be obtained by chiral HPLC separation of the reaction product); or

c) reacting a compound of formula X

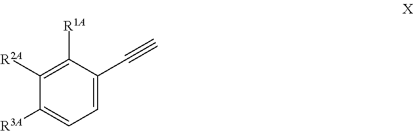

wherein R^{1A}, R^{2A} and R^{3A} have the same respective meanings as in formula I, with a compound of formula IX as defined in section b) above wherein Y^{b} represents iodine, using general reaction technique 4 leading to compounds of formula II wherein M represents M^{A} and A represents C≡C (this reaction can also be performed with racemic compound of formula IX and the (R)-enantiomer can then be obtained by chiral HPLC separation of the reaction product); or

d) reacting a compound of formula XI

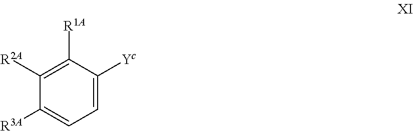

wherein R^{1A}, R^{2A} and R^{3A} have the same respective meanings as in formula I and Y^{c} represents iodine or bromine (and preferably iodine), with a compound of formula IXa

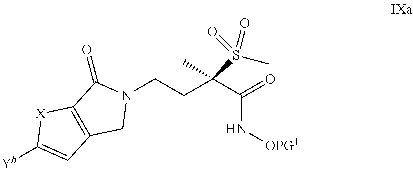

wherein Y^{b} represents ethynyl and PG¹ has the same meaning as in formula II, using general reaction technique 4 leading to compounds of formula II wherein M represents M^{A} and A represents C≡C (this reaction can also be performed with racemic compound of formula IXa and the (R)-enantiomer can then be obtained by chiral HPLC separation of the reaction product); or

e) reacting a compound of formula XII

R^{1B}≡Y^{d} XII
wherein R^{1B} has the same meaning as in formula I and Y^{d} represents iodine or bromine, with a compound of formula IXa as defined in section d) above, using general reaction technique 4 leading to compounds of formula II wherein M represents M^{B} (this reaction can also be performed with racemic compound of formula IXa and the (R)-enantiomer can then be obtained by chiral HPLC separation of the reaction product).

Preparation of the Synthesis Intermediates of Formulae VI, VII, VIII, IX, IXa, X, XI and XII:

Compounds of Formula VI:

The compounds of formula VI can be prepared as summarised in Scheme 1 hereafter.

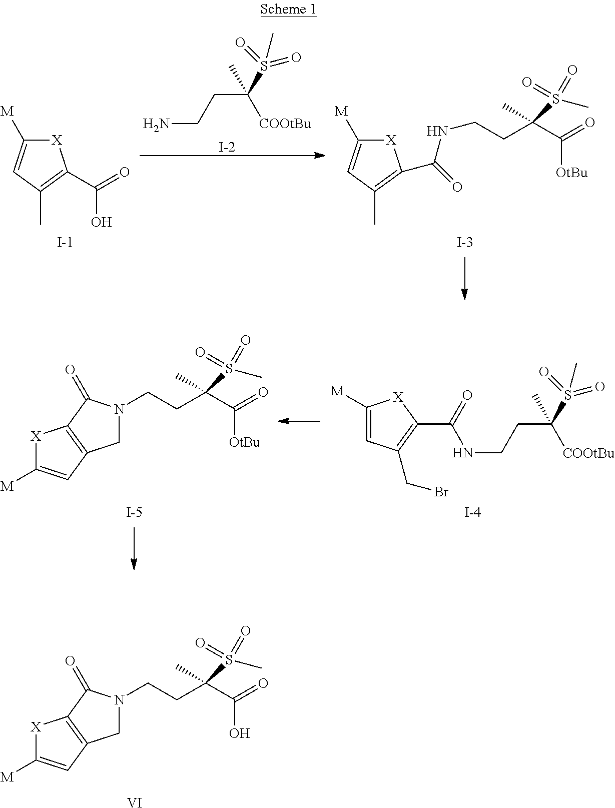

In Scheme 1, X and M have the same meaning as in formula I. The reactions can also be performed with racemic material and the (R)-enantiomer can be obtained by chiral HPLC separation at any step when suitable.

The derivatives of formula I-3 can be obtained (Scheme 1) by reaction of the thiophene carboxylic acids of formula I-1 with the amine of formula I-2 using general reaction technique 2. The derivatives of formula I-3 are treated with NBS in a solvent such as CCl₄ in the presence of a radical initiator such as AIBN; this reaction, usually performed at reflux, affords the bromo-methyl derivatives of formula I-4. The latter are subsequently transformed to the compounds of formula I-5 by treatment with a base such as LDA or LiHMDS in a solvent such as THF. The reaction can be carried out at a temperature ranging between −20° C. and rt and ideally at rt. The derivatives of formula I-5 can be transformed into the compounds of formula VI using general reaction technique 5.

Alternatively, the compounds of formula I-5 can also be prepared as summarised in Scheme 2 hereafter.

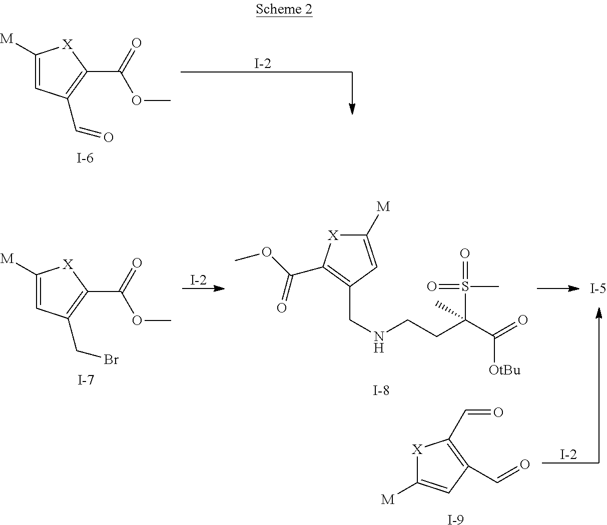

In Scheme 2, X and M have the same meaning as in formula I. The reactions can also be performed with racemic material and the (R)-enantiomer can be obtained by chiral HPLC separation at any step when suitable.

The derivatives of formula I-8 can be obtained (Scheme 2) from the derivatives of formula I-6 and the amine of formula I-2 using general reaction technique 6. Alternatively, the derivatives of formula I-8 can be obtained (Scheme 2) by treating the bromo methyl derivatives of formula I-7 with the amine of formula I-2 in tBuOH at a temperature ranging between 50° C. and 85° C., ideally at 85° C. in presence of a base such as K₂CO₃. The derivatives of formula I-8 can spontaneously give rise to the compounds of formula I-5. Alternatively, the derivatives of formula I-8 can be transformed to the derivatives of formula I-5 applying sequentially general reaction techniques 5 and 2.

The derivatives of formula I-5 can also be obtained (Scheme 2) by reaction of the dialdehydes derivatives of formula I-9 with the amine of formula I-2 in DMF at a temperature ranging between rt and about 60° C., and ideally at about 50° C. Alternatively, this condensation step can be performed in DCM in presence of AcOH at rt.

Compounds of Formula VII.

The compounds of formula VII are commercially available (PG¹=methoxymethyl, THP, TMSE, trityl, tBu, COOtBu or allyl) or can be prepared according to WO 2010/060785 (PG¹=(2-methylpropoxy)ethyl) or Marmer and Maerker, J. Org. Chem. (1972), 37, 3520-3523 (PG¹=COtBu).

Compounds of Formula VIII:

The compounds of formula VIII wherein D¹ and D² each represent H or (C₁-C₂)alkyl are commercially available or can be prepared according to Sleveland et al., Organic Process Research & Development (2012), 16, 1121-1130 starting from tri((C₁-C₂)alkyl)borate and the corresponding commercially available bromo derivatives (optionally followed by acidic hydrolysis). The compounds of formula VIII wherein D¹ and D² together represent CH₂C(Me)₂CH₂ or C(Me)₂C(Me)₂ are commercially available or can be prepared according to WO 2012/093809, starting from bis(pinacolato)diborane or 5,5-dimethyl-1,3,2-dioxaborinane (both commercially available) with the corresponding commercially available bromo derivatives of formula VIII.

Compounds of formulae IX and IXa:

The compounds of formulae IX and IXa can be prepared as summarised in Scheme 3 hereafter.

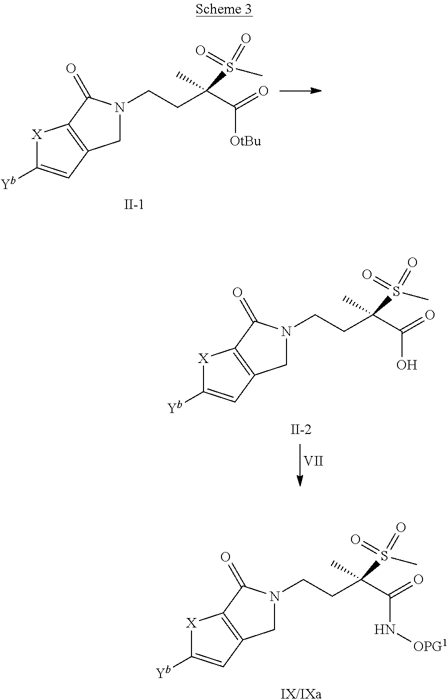

In Scheme 3, X is as defined in formula I, Y^{b} represents a halogen (such as iodine or bromine) or ethynyl and PG¹ has the same meaning as in formula II. The reactions can also be performed with racemic material and the (R)-enantiomer can be obtained by chiral HPLC separation at any step when suitable.

The compounds of formula II-1 wherein Y^{b}=Br (Scheme 3) can be transformed to the compounds of formula II-1 wherein Y^{b}=iodine by reaction with NaI in the presence of a copper (I) salt and a ligand such as trans-N,N′-dimethylcyclohexa-1,2-diamine in a solvent such as dioxane at a temperature ranging between rt and 100° C., or in a microwave oven at 150° C. The compounds of formula II-1 wherein Y^{b}=ethynyl can be can be obtained by reaction of compounds of formula II-1 wherein Y^{b}=iodine with trimethylsilylacetylene (III-1) using general reaction technique 4 followed by treatment with TBAF in THF. Alternatively the TMS group can be cleaved in MeOH using K₂CO₃ as a reagent.

The compounds of formula II-1 can be transformed to the compounds of formula II-2 using general reaction technique 5. The compounds of formula II-2 can be further reacted with the compounds of formula VII using general reaction technique 2, thus affording the compounds of formula IX/IXa. The compounds of formula IX wherein Y^{b} represents iodine can be obtained from the compounds of formula IX wherein Y^{b}=bromine by reaction with NaI in the presence of a copper (I) salt and a ligand such as trans-N,N′-dimethylcyclohexa-1,2-diamine in a solvent such as dioxane at a temperature ranging between rt and 100° C., or in a microwave oven at about 150° C. The compounds of formula IXa wherein Y^{b}=ethynyl can be can be obtained by reaction of compounds of formula IX wherein Y^{b}=iodine with trimethylsilylacetylene (III-1) using general reaction technique 4 followed by treatment with TBAF in THF. Alternatively the TMS group can be cleaved in MeOH using K₂CO₃ as a reagent.

Compounds of Formula X

The compounds of formula X are commercially available or can be prepared as summarised in Scheme 4 hereafter.

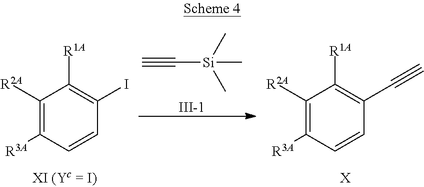

In Scheme 4, R^{1A}, R^{2A} and R^{3A} have the same respective meanings as in formula I.

The compounds of formula XI wherein Y^{c} represents iodine can be reacted (Scheme 4) with trimethylsilylacetylene (III-1) using general reaction technique 4 followed by treatment with TBAF in THF, affording the derivatives of formula X.

Compounds of Formula XI:

The compounds of formula XI wherein Y^{c} represents bromine are commercially available or can be prepared by standard methods known to one skilled in the art. The compounds of formula XI wherein Y^{c} represents iodine can be obtained from the corresponding bromine derivatives by reaction with NaI in the presence of a copper (I) salt and a ligand such as trans-N,N′-dimethylcyclohexa-1,2-diamine in a solvent such as dioxane at a temperature ranging between rt and 100° C., or in a microwave oven at about 150° C.

Compounds of Formula XII:

The compounds of formula XII wherein Y^{d} represents iodine can be prepared by iodination of the corresponding compounds wherein Y^{d} would be H with iodine in the presence of an inorganic base such as KOH. The compounds of formula XII wherein Y^{d} represents bromine can be prepared from the corresponding compounds wherein Y^{d} would be H by treatment with NBS in the presence of AgNO₃ in a solvent such as acetone or MeCN. The compounds of formula XII wherein Y^{d} would be H are commercially available or can be prepared by standard methods known to one skilled in the art.

Other Synthesis Intermediates and Starting Materials:

The compounds of formula I-1, I-6, I-7 and I-9 are commercially available or can be prepared by standard methods known to one skilled in the art.

The compound of formula I-2 can be prepared as described in the section entitled “EXAMPLES” hereafter (see Preparations A and B), or by standard methods known to one skilled in the art.

The compounds of formula II-1 wherein Y^{b} represents Br can be prepared as summarised in Scheme 5 hereafter.

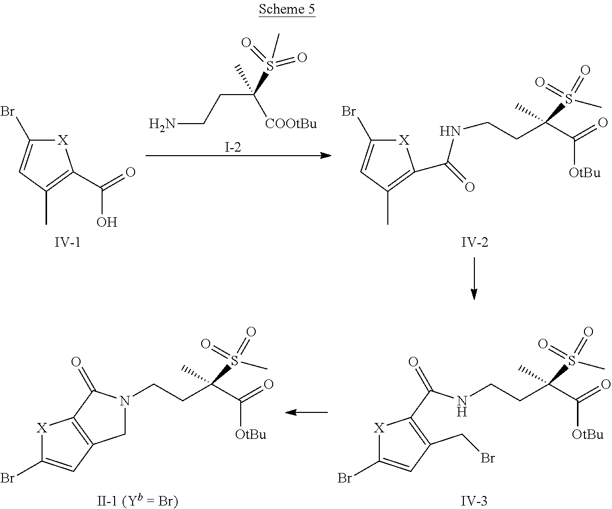

In Scheme 5, X is as defined in formula I.

The compounds of formula IV-I can be transformed (Scheme 5) to the amide derivatives of formula IV-2 by reaction with the amine of formula I-2 using general reaction technique 2. The resulting derivatives of formula IV-2 can be transformed to the derivatives of formula IV-3 by treatment with NBS in a solvent such as CCl₄ in the presence of a radical initiator such as AIBN; this reaction is usually performed at reflux. The resulting bromo derivatives of formula IV-3 is subsequently transformed to the compounds of formula II-1 wherein Y^{b} is Br by treatment with a base such as LDA or LiHMDS in a solvent such as THF.

The compounds of formula II-1 wherein Y^{b} is bromine can also be prepared as summarised in Scheme 6 hereafter.

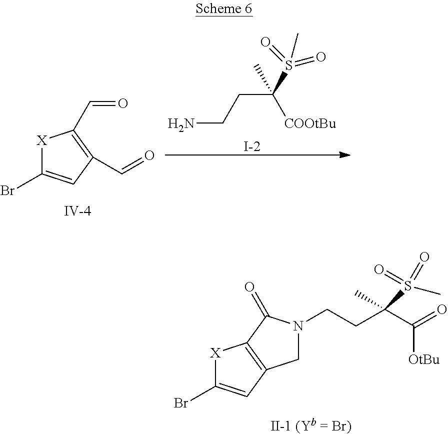

In Scheme 6, X is as defined in formula I.

The compounds of formula II-1 can be obtained (Scheme 6) by reaction of the compounds of formula IV-4 with the amine of formula I-2 in DCM in presence of acetic acid at a temperature ranging between rt and 40° C., and ideally at rt. The reaction can also be performed with racemic material and the (R)-enantiomer can be obtained by chiral HPLC separation at any step when suitable.

The compounds of formula III, IV and V are commercially available or can be prepared by standard methods known to one skilled in the art.

The compounds of formula IV-1 and IV-4 are commercially available or can be prepared by standard methods known to one skilled in the art.

Particular embodiments of the invention are described in the following Examples, which serve to illustrate the invention in more detail without limiting its scope in any way.

### EXAMPLES

All temperatures are stated in ° C. Unless otherwise indicated, the reactions take place at rt. The combined org. layers resulting from the liquid-liquid extraction during the work-up procedure of a reaction mixture are, unless otherwise indicated, washed with a minimal volume of brine, dried over MgSO₄, filtered and evaporated to dryness to provide a so-called evaporation residue.

Analytical TLC characterisations were performed with 0.2 mm plates: Merck, Silica gel 60 F₂₅₄. Elution is performed with EA, Hept, DCM, MeOH or mixtures thereof. Detection was done with UV or with a solution of KMnO₄ (3 g), K₂CO₃ (20 g), 5% NaOH (3 mL) and H₂O (300 mL) with subsequent heating.

CCs were performed using Brunschwig 60A silica gel (0.032-0.63 mm) or using an ISCO CombiFlash system and prepacked SiO₂ cartridges, elution being carried out with either Hept-EA or DCM-MeOH mixtures with an appropriate gradient. When the compounds contained an acid function, 1% of AcOH was added to the eluent(s). When the compounds contained a basic function, 25% aq. NH₄OH was added to the eluents.

The compounds were characterized by ¹H-NMR (300 MHz, Varian Oxford; 400 MHz, Bruker Avance 400 or 500 MHz, Bruker Avance 500 Cryoprobe). Chemical shifts 6 are given in ppm relative to the solvent used; multiplicities: s=singlet, d=doublet, t=triplet, q=quartet, p=pentet, hex=hexet, hep=heptet, m=multiplet, br=broad; coupling constants J are given in Hz. Alternatively or additionally compounds were characterized by LC-MS (Sciex API 2000 with Agilent 1100 Binary Pump with DAD and ELSD or an Agilent quadrupole MS 6140 with Agilent 1200 Binary Pump, DAD and ELSD).

The analytical LC-MS data have been obtained using the following respective conditions:
Column: Zorbax SB-Aq, 30.5 μm, 4.6×50 mm;
Injection volume: 1 μL;
Column oven temperature: 40° C.;
Detection: UV 210 nm, ELSD and MS;
MS ionization mode: ESI+;
Eluents: A: H₂O+0.04% TFA; and B: MeCN;
Flow rate: 40.5 mL/min;
Gradient: 5% B to 95% B (0.0 min-1.0 min), 95% B (1.0 min-1.45 min).

The number of decimals given for the corresponding [M+H⁺] peak(s) of each tested compound depends upon the accuracy of the LC-MS device actually used.

The prep-HPLC purifications were performed on a Gilson HPLC system, equipped with a Gilson 215 autosampler, Gilson 333/334 pumps, Dionex MSQ Plus detector system, and a Dionex UVD340U (or Dionex DAD-3000) UV detector, using the following respective conditions:
Method 1:
   Column: Waters XBridge C18, 10 μm, 30×75 mm;
   Flow rate: 75 mL/min;
   Eluents: A: H₂O+0.5% NH₄OH solution (25%); B: MeCN;
   Gradient: 90% A to 5% A (0.0 min-4.0 min), 5% A (4.0 min-6.0 min).
Method 2:
   Column: Waters XBridge C18, 10 μm, 30×75 mm;
   Flow rate: 75 mL/min;
   Eluents: A: H₂O+0.5% HCOOH; B: MeCN;
   Gradient: 90% A to 5% A (0.0 min-4.0 min), 5% A (4.0 min-6.0 min).
Method 3:
   Column: Waters Atlantis T3 OBD, 10 μm, 30×75 mm;
   Flow rate: 75 mL/min;
   Eluents: A: H₂O+0.1% HCOOH; B: MeCN+0.1% HCOOH;
   Gradient: 90% A to 5% A (0.0 min-4.0 min), 5% A (4.0 min-6.0 min).

Besides, semi-preparative and analytical chiral HPLCs were performed using the conditions hereafter.

Semi-Preparative Chiral HPLC Method A:

The semi-preparative chiral HPLC is performed on a Daicel ChiralPak AS-H column (250×20 mm, 20 μm) using the eluent mixture, flow rate and detection conditions indicated between brackets in the corresponding experimental protocol. The retention times are obtained by elution of analytical samples on a Daicel ChiralPak AS-H column (250×4.6 mm, 5 μm) using the same eluent mixture with the flow rate indicated between brackets in the corresponding experimental protocol.

Semi-Preparative Chiral HPLC Method B:

The semi-preparative chiral HPLC is performed on a Daicel ChiralCel OD-H column (20×250 mm; 5 μm) using the eluent mixture, flow rate and detection conditions indicated between brackets in the corresponding experimental protocol. The retention times are obtained by elution of analytical samples on a Daicel ChiralCel OD-H column (4.6×250 mm; 5 μm) using the same eluent mixture with the flow rate indicated between brackets in the corresponding experimental protocol.

Semi-Preparative Chiral HPLC Method C:

The semi-preparative chiral HPLC is performed on a Daicel ChiralPak AY-H column (20×250 mm, 5 μm) using the eluent mixture, flow rate and detection conditions indicated between brackets in the corresponding experimental protocol. The retention times are obtained by elution of analytical samples on a Daicel ChiralPak AY-H column (4.6×250 mm, 5 μm) using the same eluent mixture with the flow rate indicated between brackets in the corresponding experimental protocol.

Semi-Preparative Chiral HPLC Method D:

The semi-preparative chiral HPLC is performed on a Daicel ChiralPak IA column (30×250 mm, 5 μm) using the eluent mixture, flow rate and detection conditions indicated between brackets in the corresponding experimental protocol. The retention times are obtained by elution of analytical samples on a Daicel ChiralPak IA column (4.6×250 mm, 5 μm) using the same eluent mixture with the flow rate indicated between brackets in the corresponding experimental protocol.

Analytical Chiral HPLC Method E:

The retention times are obtained by elution of analytical samples on a Daicel ChiralPak AD-H column (4.6×250 mm, 5 μm) using the same eluent mixture with the flow rate indicated between brackets in the corresponding experimental protocol.

Semi-Preparative Chiral HPLC Method F:

The semi-preparative chiral HPLC is performed on a Daicel ChiralPak IC column (30×250 mm, 5 μm) using the eluent mixture, flow rate and detection conditions indicated between brackets in the corresponding experimental protocol. The retention times are obtained by elution of analytical samples on a Daicel ChiralPak IC column (4.6×250 mm, 5 μm) using the same eluent mixture with the flow rate indicated between brackets in the corresponding experimental protocol.

Procedures:

Procedure A:

CuI (0.048 mmol), PdCl₂(PPh₃)₂ (0.025 mmol), the iodo derivative (0.148 mmol) and the terminal alkyne (0.16 mmol) are introduced in a two-necked round flask. The atmosphere is flushed with nitrogen during 30 min, then degassed THF (1.2 mL) and degassed TEA (0.43 mmol) are added. The suspension is stirred under nitrogen atmosphere at 50° C. for 3 h. After concentration to dryness, the residue is then purified by CC (Hept-EA or DCM-MeOH) or by prep-HPLC using a suitable method.

Procedure B:

To the THP-protected hydroxamic acid derivative (0.07 mmol) in EtOH (2 mL) is added PPTS (0.012 g; 0.04 mmol). The mixture is stirred at 80° C. for 2 h, cooled to rt and directly purified by CC (DCM-MeOH) or by prep-HPLC using a suitable method.

Procedure C:

CuI (0.036 g; 0.189 mmol), PdCl₂(PPh₃)₂ (0.072 g; 0.102 mmol), (trimethylsilyl)ethynyl acetylene (0.703 mmol) and the halo derivative (0.581 mmol) are introduced in a two-necked round flask. The atmosphere is flushed with nitrogen during 30 min, then degassed THF (4 mL) and degassed TEA (0.2 mL; 1.44 mmol) are added. The suspension is stirred under nitrogen atmosphere at 50° C. for 2 h. After concentration to dryness, the residue is then purified by CC (Hept-EA).

Procedure D:

CuCl (0.003 g; 0.026 mmol) was added to a solution of n-BuNH₂ (30% in water, 0.336 mL). NH₂OH.HCl (0.025 g, 0.35 mmol) and a solution of alkyne (0.23 mmol) in n-BuNH₂ (30% in water, 1 mL) were successively added. The resulting suspension was immediately cooled with an ice-water bath. A solution of halo-alkyne (0.25 mmol) in Et₂O (0.5 mL) was added and the reaction mixture was allowed to proceed for 1.5 h. The evaporation residue is then purified by CC (DCM-MeOH) or by prep-HPLC using a suitable method to afford the bis-alkyne product.

Procedure E:

A mixture of the bromo derivative (1.63 mmol), the phenylboronic acid or boronate ester derivative (1.8 mmol), K₂CO₃ (0.34 g; 2.4 mmol) and Pd(PPh₃)₄ (0.19 g; 0.16 mmol) is flushed with nitrogen for 15 min. Dioxane (6 mL) and water (1.5 mL) are added and the mixture is refluxed for 1 h. After cooling, water (15 mL) and EA (20 mL) are added and the two layers are separated. The aq. layer is extracted with EA (2×20 mL) and the combined org. layers are washed with brine, dried over MgSO₄ and concentrated to dryness. The residue is then purified by CC (Hept-EA).

Procedure F:

To a solution of the THP-protected hydroxamic acid derivative (0.15 mmol) in MeOH (1.2 mL) and water (0.4 mL) is added 2M HCl (0.6 mL; 1.2 mmol). The reaction mixture is stirred until completion. The reaction mixture, after neutralization with sat. NaHCO₃ solution, is extracted with DCM-MeOH (9-1, 3×20 mL). The evaporation residue is then purified by CC (DCM-MeOH) or by prep-HPLC using a suitable method.

Procedure G:

CuI (0.2 mmol), PdCl₂(PPh₃)₂ (0.1 mmol), the terminal alkyne derivative (1 mmol) and the iodo derivative (1.2 mmol) are introduced in a two necked round flask. The atmosphere is flushed with nitrogen during 30 min, then degassed THF (5 mL) and degassed TEA (2.5 mmol) are added. The suspension is stirred under nitrogen atmosphere at 50° C. for 45 min. After concentration to dryness, the residue is then purified by CC (Hept-EA) or by prep-HPLC using a suitable method.

### PREPARATIONS

### Preparation A: (2RS)-tert-butyl 4-amino-2-methyl-2-(methylsulfonyl)butanoate

### A.i. (RS)-tert-butyl 2-(methylsulfonyl)propanoate

To a suspension of sodium methanesulfinate (100 g; 929 mmol) in tBuOH (350 mL) was added tert-butyl-2-bromopropionate (150 mL; 877 mmol). The reaction mixture was stirred at 90° C. for 24 h under nitrogen atmosphere, then cooled to rt and concentrated to dryness. The residue was partitioned between water (750 mL) and EA (600 mL). The aq. layer was extracted with EA (2×500 mL). The evaporation residue afforded the title compound as a white yellow solid (175 g, 96% yield).

¹H NMR (d₆-DMSO) δ: 4.24 (q, J=7.2 Hz, 1H); 3.11 (s, 3H); 1.45 (s, 9H); 1.40 (d, J=7.2 Hz, 3H).

### A.ii. (2RS)-tert-butyl 4-bromo-2-methyl-2-(methylsulfonyl)butanoate

To an ice-chilled suspension of intermediate A.i (130 g; 626 mmol) in DMF (750 mL) was added portionwise NaH (60% in mineral oil; 32.1 g; 802 mmol) for 1.5 h, keeping the temperature below 7° C. The mixture was stirred at 0° C. for 1.5 h, allowed to reach rt and stirred for 0.5 h. The mixture was cooled down to 12° C. with an ice bath and 1,2-dibromoethane (166 mL; 1.9 mol) was then added dropwise, keeping the temperature below 22° C. The reaction mixture was stirred for 2 h. The mixture was poured into cold water (1 L) and Et₂O (1 L) and the aq. layer was extracted with Et₂O (2×750 mL). The org. layer was washed with cold water (2×500 mL). The evaporation residue was purified by CC (Hept-EA) to afford the title compound as a pale yellowish oil (116.8 g; 59% yield).

¹H NMR (d₆-DMSO) δ: 3.71-3.63 (m, 1H); 3.45-3.37 (m, 1H); 3.12 (s, 3H); 2.72-2.62 (m, 1H); 2.43-2.33 (m, 1H); 1.49 (s, 3H); 1.46 (s, 9H).

### A.iii. (2RS)-tert-butyl 4-azido-2-methyl-2-(methylsulfonyl)butanoate

To a solution of intermediate A.ii (70.3 g; 223 mmol) in DMF (400 mL) was added sodium azide (54.6 g; 831 mmol). The reaction was stirred at 80° C. overnight. The mixture was cooled to rt and water (500 mL) and EA (500 mL) were added. The aq. layer was extracted with EA (2×500 mL) and the org. layer was washed with water (2×500 mL). The evaporation residue was triturated in Hept, filtered and washed with Hept to afford the title compound as a white solid (59.6 g; 96% yield).

¹H NMR (d₆-DMSO) δ: 3.66-3.60 (m, 1H); 3.35-3.29 (overlapped m, 1H); 3.11 (s, 3H); 2.49-2.43 (m, 1H); 2.04-1.96 (m, 1H); 1.46 (s, 9H); 1.44 (s, 3H).

MS (ESI, m/z): 278.95 [M+H⁺] for C₁₀H₁₉N₃O₄S; t_{R}=0.80 min.

### A. iv. (2RS)-tert-butyl 4-amino-2-methyl-2-(methylsulfonyl)butanoate

A solution of intermediate A.iii (45 g; 162 mmol) in a mixture of tBuOH/EA (1/1, 900 mL) was treated with 10% Pd/C (2.3 g). The suspension was stirred under hydrogen for 4 h. Then 10% Pd/C (0.5 g) was added to the suspension and the reaction was stirred under hydrogen for 2 days. The catalyst was filtered off and the filtrate concentrated to dryness to afford the crude material which crystallized on standing (grey solid; 40.6 g; 99% yield).

¹H NMR (d₆-DMSO) δ: 3.06 (s, 3H); 2.75-2.63 (m, 1H); 2.53-2.40 (overlapped m, 1H); 2.28-2.16 (m, 1H); 1.85-1.74 (m, 1H); 1.44 (s, 9H); 1.40 (s, 3H).

MS (ESI, m/z): 252.03 [M+H⁺] for C₁₀H₂₁NO₄S; t_{R}=0.45 min.

### Preparation B: (2R)-tert-butyl 4-amino-2-methyl-2-(methylsulfonyl)butanoate

### B.i. (2R)-tert-butyl 4-azido-2-methyl-2-(methylsulfonyl)butanoate

Intermediate A.iii (184 g) was separated by semi-preparative chiral HPLC Method A (Hept-iPrOH 4-1; flow rate: 570 mL/min; UV detection at 235 nM); the respective retention times were 8.3 and 10.7 min. The title (R)-enantiomer, identified as the second eluting compound, was obtained as a light orange oil (90.7 g).

¹H NMR (d₆-DMSO) δ: 3.66-3.60 (m, 1H); 3.35-3.29 (overlapped m, 1H); 3.11 (s, 3H); 2.50-2.43 (overlapped m, 1H); 2.04-1.97 (m, 1H); 1.46 (s, 9H); 1.44 (s, 3H).

B.ii. (2R)-tert-butyl 4-amino-2-methyl-2-(methylsulfonyl)butanoate:

Starting from intermediate B.i (45 g; 162 mmol) and proceeding in analogy to Preparation A, step A.iv, the title compound was obtained as grey solid (40.6 g; 99% yield).

¹H NMR (d₆-DMSO) δ: 3.06 (s, 3H); 2.75-2.63 (m, 11H); 2.53-2.40 (overlapped m, 1H); 2.28-2.16 (m, 1H); 1.85-1.74 (m, 1H); 1.44 (s, 9H); 1.40 (s, 3H).

MS (ESI, m/z): 252.03 [M+H⁺] for C₁₀H₂₁NO₄S; t_{R}=0.45 min.

### Preparation C: tert-butyl (2RS)-2-methyl-2-(methylsulfonyl)-4-(6-oxo-4,6-dihydro-5H-thieno[2,3-c]pyrrol-5-yl)butanoate

### C.i. Tert-butyl (2RS)-2-methyl-2-(methylsulfonyl)-4-(3-methylthiophene-2-carboxamido)butanoate

To a solution of the compound of Preparation A (6.91 g; 27.5 mmol) and 3-methyl-2-thiophenecarboxylic acid (3.97 g; 27.4 mmol) in DMF (150 mL), were added EDC (10.31 g; 53.2 mmol), HOBT (7.59 g; 55 mmol) and TEA (11 mL, 78.9 mmol). The reaction mixture was stirred overnight. After concentration to dryness, the residue was diluted with H₂O (50 mL) and EA (60 mL). The two layers were separated and the org. layer was washed with 15% aq. NaHSO₄ (40 mL) and sat. aq. NaHCO₃ (40 mL). The evaporation residue afforded a yellow oil which crystallized on standing (10.3 g, 100% yield).

¹H NMR (d6-DMSO) δ: 8.01 (t, J=5.6 Hz, 1H), 7.56 (d, J=5.0 Hz, 1H), 6.95 (d, J=5.0 Hz, 1H), 3.29 (m, 1H), 3.23 (m, 1H), 3.12 (s, 3H), 2.41 (s, 3H), 2.35 (m, 1H), 1.99 (m, 1H), 1.49 (s, 3H), 1.44 (s, 9H).

MS (ESI, m/z): 375.91 [M+H⁺] for C₁₆H₂₅NO₅S₂; t_{R}=0.81 min.

### C.ii. Tert-butyl (RS)-4-(3-(bromomethyl)thiophene-2-carboxamido)-2-methyl-2-(methylsulfonyl)butanoate

A mixture of intermediate C.i (3.18 g; 8.47 mmol), NBS (1.83 g; 10.2 mmol) and AIBN (0.140 g; 0.85 mmol) in CCl₄ (60 mL) was heated under reflux overnight. The solution was allowed to cool to rt, diluted with DCM (70 mL) and washed with water (60 mL). The evaporation residue afforded the title compound (3.33 g; 86% yield) as a yellowish gum.

¹H NMR (d6-DMSO) δ: 8.37 (t, J=5.6 Hz, 1H), 7.67 (d, J=5.1 Hz, 1H), 7.20 (d, J=5.1 Hz, 1H), 4.99 (s, 2H), 3.37-3.29 (overlapped m, 1H), 3.27-3.19 (m, 1H), 3.12 (s, 3H), 2.39-2.32 (m, 1H), 2.06-1.97 (m, 1H), 1.49 (s, 3H), 1.45 (s, 9H).

MS (ESI, m/z): 455.87 [M+H⁺] for C₁₆H₂₄NO₅BrS₂; t_{R}=0.86 min.

### C.iii. Tert-butyl (2RS)-2-methyl-2-(methylsulfonyl)-4-(6-oxo-4,6-dihydro-5H-thieno[2,3-c]pyrrol-5-yl)butanoate

To a solution of the intermediate C.ii (3.33 g; crude) in THF (65 mL), cooled to −20° C., was added LiHMDS (1M in THF, 8.6 mL; 8.6 mmol) dropwise over 5 min. The reaction mixture was stirred for 3 h. The reaction mixture was quenched adding 10% aq. NaHSO₄ (50 mL) and extracted with EA (3×40 mL). The evaporation residue was purified by CC (Hept-EA gradient) to afford the title compound (0.562 g; 18% yield) as a yellow solid.

¹H NMR (d6-DMSO) δ: 7.97 (d, J=4.8 Hz, 1H), 7.24 (d, J=4.8 Hz, 1H), 4.47-4.36 (m, 2H), 3.63 (m, 1H), 3.51 (m, 1H), 3.12 (s, 3H), 2.43 (m, 1H), 2.04 (m, 1H), 1.53 (s, 3H), 1.34 (s, 9H).

MS (ESI, m/z): 373.8 [M+H⁺] for C₁₆H₂₃NO₅S₂; t_{R}=0.76 min.

### Preparation D: (2RS)-4-(2-bromo-6-oxo-4,6-dihydro-5H-thieno[2,3-c]pyrrol-5-yl)-2-methyl-2-(methylsulfonyl)-N—(((RS)-tetrahydro-2H-pyran-2-yl)oxy)butanamide

### D.i. Methyl (3RS)-5-bromo-3-(((4-(tert-butoxy)-3-methyl-3-(methylsulfonyl)-4-oxobutyl)amino)methyl)thiophene-2-carboxylate

A mixture of methyl 5-bromo-3-(bromomethyl)thiophene-2-carboxylate (1.38 g; 4.42 mmol), the compound of Preparation A (1.05 g; 4.18 mmol) and K₂CO₃ (0.683 g; 4.94 mmol) in tBuOH (90 mL) was stirred at reflux overnight. The reaction mixture was concentrated in vacuo and the residue was purified by CC (DCM-MeOH containing 1% of aq. NH₄OH) to afford the pure title compound as a yellow gum (1.06 g; 50% yield).

¹H NMR (d₆-DMSO) δ: 7.38 (s, 1H); 3.93 (s, 2H); 3.79 (s, 3H); 3.06 (s, 3H); 2.60 (m, 1H); 2.42-2.27 (m, 2H); 1.82 (m, 1H); 1.39 (s, 9H); 1.38 (s, 3H).

MS (ESI, m/z): 485.76 [M+H⁺] for C₁₇H₂₆NO₆BrS₂; t_{R}=0.72 min.

### D.ii. (3RS)-5-bromo-3-(((4-(tert-butoxy)-3-methyl-3-(methylsulfonyl)-4-oxobutyl)amino)methyl)thiophene-2-carboxylic acid

LiOH.H₂O (0.290 g; 3.86 mmol) was added to a solution of intermediate D.i (1.06 g; 2.2 mmol) in THF-MeOH-water (2-2-1, 20 mL). The reaction was stirred for 2.5 h. Solvents were evaporated under vacuum to afford the crude product (1.35 g; >95% yield) as a yellow gum. The product was used without further purification.

MS (ESI, m/z): 471.9 [M+H⁺] for C₁₆H₂₄NO₆BrS₂; t_{R}=0.66 min.

### D.iii. (2RS)-tert-butyl 4-(2-bromo-6-oxo-4,6-dihydro-5H-thieno[2,3-c]pyrrol-5-yl)-2-methyl-2-(methylsulfonyl)butanoate

To a solution of intermediate D.ii (1.35 g, crude) in DMF (25 mL) were added EDC (0.863 g; 4.46 mmol), HOBT (0.615 g; 4.46 mmol) and TEA (0.885 mL; 6.35 mmol). The resulting mixture was stirred overnight. The reaction mixture was concentrated to dryness. The residue was taken up in H₂O (30 mL) and EA (40 mL). The layers were separated. The org. layer was washed with 15% aq. NaHSO₄ (25 mL), sat. aq. NaHCO₃ (25 mL) and brine (20 mL). The evaporation residue was purified by CC (Hept-EA gradient) to afford the title compound (0.145 g; 15% yield) as a yellow gum.

¹H NMR (d6-DMSO) δ: 7.48 (s, 1H), 4.46-4.37 (m, 2H), 3.59 (m, 1H), 3.50 (m, 1H), 3.10 (s, 3H), 2.56-2.46 (overlapped m, 1H), 2.02 (m, 1H), 1.51 (s, 3H), 1.35 (s, 9H).

MS (ESI, m/z): 453.75 [M+H⁺] for C₁₆H₂₂NO₅BrS₂; t_{R}=0.85 min.

### D.iv. (2RS)-4-(2-bromo-6-oxo-4,6-dihydro-5H-thieno[2,3-c]pyrrol-5-yl)-2-methyl-2-(methylsulfonyl)butanoic Acid

Variant A:

To a mixture of the intermediate D.iii (0.153 g; 0.34 mmol) in 4N HCl in dioxane (3.2 mL) was added H₂O (0.078 mL). The reaction mixture was stirred overnight. The reaction mixture was concentrated to dryness, then co-evaporated with Et₂O (10 mL) to give the title acid (0.155 g; quant.) as a yellow solid.

¹H NMR (d6-DMSO) δ: 7.47 (s, 1H); 4.45-4.36 (m, 2H); 3.68-3.61 (m, 1H); 3.54-3.48 (m, 1H); 3.11 (s, 3H); 2.58-2.46 (overlapped m, 1H); 2.07-1.98 (m, 1H); 1.53 (s, 3H).

MS (ESI, m/z): 397.76 [M+H⁺] for C₁₂H₁₄NO₅BrS₂; t_{R}=0.64 min.

Alternatively, the title compound was prepared as follows:

Variant B:

To a solution of the compound of Preparation C (0.557 g; 1.49 mmol) in AcOH (30 mL) were added successively benzyltrimethylammonium tribromide (3.01 g; 7.48 mmol) and ZnCl2 (0.748 g; 5.48 mmol). The resulting mixture was stirred overnight. The reaction mixture was quenched by addition of 15% aq. NaHSO₄ (25 mL) and extracted with EA (3×30 mL). The evaporation residue was further co-evaporated with cyclohexane (3×25 mL) to afford the title compound (2.73 g, contaminated with benzyltrimethylammonium salts).

MS (ESI, m/z): 397.7 [M+H⁺] for C₁₂H₁₄NO₅BrS₂; t_{R}=0.64 min (Zorbax).

### D.v. (2RS)-4-(2-bromo-6-oxo-4,6-dihydro-5H-thieno[2,3-c]pyrrol-5-yl)-2-methyl-2-(methylsulfonyl)-N-(((2RS)-tetrahydro-2H-pyran-2-yl)oxy)butanamide

To a solution of intermediate D.iv (0.155 g; crude) in DMF (1.4 mL) were added successively HOBT (0.099 g; 0.71 mmol), TEA (0.141 mL; 1.01 mmol), THP-ONH₂ (0.099 g; 0.83 mmol) and EDC (0.128 g; 0.66 mmol). The reaction mixture was diluted with DMF (0.7 mL) and the suspension was stirred overnight. The reaction mixture was concentrated in vacuo and partitioned between H₂O (10 mL) and EA (10 mL). The org. layer was washed with aq. 15% NaHSO₄ (10 mL) and aq. sat. NaHCO₃ (10 mL). The evaporation residue was purified by CC using a Hept-EA gradient to afford the title compound as a yellow gum (0.073 g, 44% yield).

¹H NMR (d6-DMSO) δ: 11.35 (s, 0.5H); 11.32 (s, 0.5H); 7.47 (s, 0.5H); 7.46 (s, 0.5H); 4.88-4.84 (m, 0.5H); 4.61-4.58 (m, 0.5H); 4.49-4.36 (m, 2H); 4.08-3.92 (m, 1H); 3.59-3.49 (m, 2H); 3.46-3.40 (m, 1H); 3.06 (s, 1.5H); 3.03 (s, 1.5H); 2.65-2.47 (overlapped m, 1H); 2.00-1.91 (n, 1H); 1.68-1.60 (m, 3H); 1.59-1.45 (overlapped m, 3H); 1.56 (s, 1.5H); 1.54 (s, 1.5H).

MS (ESI, m/z): 496.9 [M+H⁺] for C₁₇H₂₃N₂O₆BrS₂; t_{R}=0.74 min.

### Preparation E: 2-(4-ethynylphenyl)-2-methylpropan-1-ol

In a tube, 2-(4-bromophenyl)-2-methylpropan-1-ol (0.742 g; 3.2 mmol; commercial), bis(tri-tert-butylphosphine) palladium (0.141 g; 0.276 mmol) and CsF (0.981 g; 6.47 mmol) were degassed. Dioxane (18 mL) was degassed and added simultaneously with ethynyltri-n-butyltin (1.4 mL, 4.83 mmol) into the tube. The mixture was degassed three times and the reaction was stirred at 80° C. for 1.5 h. After evaporation to dryness, the mixture was purified by CC (Hept-EA gradient) to afford the title compound as a ocre solid. (0.539 g; 96% yield).

¹H NMR (d₆-DMSO): 7.42-7.33 (m, 4H); 4.69 (t, J=5.4 Hz, 1H); 4.09 (s, 1H); 3.40 (d, J=5.4 Hz, 2H); 1.20 (s, 6H).

### Preparation F: (2RS)-4-(2-ethynyl-6-oxo-4,6-dihydro-5H-thieno[2,3-c]pyrrol-5-yl)-2-methyl-2-(methylsulfonyl)-N-(((2RS)-tetrahydro-2H-pyran-2-yl)oxy)butanamide

### F.i. (2RS)-2-methyl-2-(methylsulfonyl)-4-(6-oxo-2-((trimethylsilyl)ethynyl)-4,6-dihydro-5H-thieno[2,3-c]pyrrol-5-yl)-N-(((2RS)-tetrahydro-2H-pyran-2-yl)oxy)butanamide

Starting from the compound of Preparation D (0.288 g; 0.58 mmol) and proceeding in analogy to Procedure C, the title compound (0.189 g, 64% yield) was prepared as a brown solid.

¹H NMR (d6-DMSO) δ: 11.35 (s, 0.5H); 11.33 (s, 0.5H); 7.48 (s, 0.5H); 7.47 (s, 0.5H); 4.86 (m, 0.5H); 4.65 (m, 0.5H); 4.51-4.36 (m, 2H); 4.07-3.94 (m, 1H); 3.63-3.37 (m, 3H); 3.06 (s, 1.5H); 3.03 (s, 1.5H); 2.67-2.46 (overlapped m, 1H); 1.98 (m, 1H); 1.69-1.45 (overlapped m, 6H); 1.55 (s, 1.5H); 1.54 (s, 1.5H); 0.25 (m, 9H).

MS (ESI, m/z): 513.0 [M+H⁺] for C₂₂H₃₂N₂O₆S₂Si; t_{R}=0.90 min.

### F.ii. (2RS)-4-(2-ethynyl-6-oxo-4,6-dihydro-5H-thieno[2,3-c]pyrrol-5-yl)-2-methyl-2-(methylsulfonyl)-N-(((2RS)-tetrahydro-2H-pyran-2-yl)oxy)butanamide

To a solution of intermediate F.i (0.189 g; 0.37 mmol) in MeOH (3.4 mL) was added K₂CO₃ (0.080 g; 0.58 mmol). The reaction mixture was stirred for 1 h. The reaction mixture was diluted with water (20 mL) and EA (40 mL). The phases were separated and the aq. layer was extracted with EA-MeOH (9-1, 5×20 mL). The combined org. layers were dried over MgSO₄, filtered and evaporated under reduced pressure to give the title compound (0.164 g, >95% yield) as a brown gum.

¹H NMR (d6-DMSO) δ: 11.35 (s, 0.5H); 11.32 (s, 0.5H); 7.50 (s, 0.5H); 7.50 (s, 0.5H); 4.88 (s, 1H); 4.86 (m, 0.5H); 4.61 (m, 0.5H); 4.49-4.36 (m, 2H); 4.02 (m, 1H); 3.62-3.50 (m, 2H); 3.43 (m, 1H); 3.06 (s, 1.5H); 3.03 (s, 1.5H); 2.66-2.45 (overlapped m, 1H); 1.99 (m, 1H); 1.69-1.44 (overlapped m, 6H); 1.56 (s, 1.5H); 1.55 (s, 1.5H).

MS (ESI, m/z): 440.93 [M+H⁺] for C₁₉H₂₄N₂O₆S₂; t_{R}=0.72 min.

### Preparation G: ((1-(bromoethynyl)cyclopropyl)methoxy)(tert-butyl)diphenylsilane

To a mixture of (dibromomethyl)triphenylphosphonium bromide (8.527 g; 16.6 mmol) and THF (40 mL) was added a solution of tBuOK (1M in THF; 16.6 mL; 16.6 mmol). The resulting dark brown solution was stirred for 3 min, then cooled to 0° C. A solution of 1-(((tert-butyldiphenylsilyl)oxy)methyl)cyclopropanecarbaldehyde (2.2 g; 6.62 mmol; prepared as described in WO 2010/135536) in THF (23 mL) was added dropwise. The reaction was stirred at 0° C. for 40 min. The reaction mixture was cooled to −78° C. and tBuOK (1M in THF; 29.1 mL; 29.1 mmol) was added rapidly and stirred at −78° C. for 30 min. The reaction mixture was quenched with brine (150 mL). The aq. layer was separated and extracted with Et₂O (3×150 mL). The evaporation residue was purified by CC (Hept-EA) to afford the title compound as a colourless oil (2.05 g; 75% yield).

¹H NMR (d6-DMSO) δ: 7.70-7.59 (m, 4H); 7.53-7.37 (m, 6H); 3.56 (s, 2H); 1.01 (s, 9H); 0.89-0.82 (m, 2H); 0.76-0.71 (m, 2H).

### Preparation H: ((1S,2S)-2-(bromoethynyl)cyclopropyl)methyl Acetate AND ((1R,2R)-2-(bromoethynyl)cyclopropyl)methyl Acetate

H.i. ((1S*,2S*)-2-(2,2-dibromovinyl)cyclopropyl)methyl acetate:

To a solution of CBr₄ (30.0 g; 88.9 mmol) in DCM (60 mL) cooled at −20° C., was added dropwise over 45 min a solution of PPh₃ (45.8 g, 175 mmol) in DCM (100 mL). The mixture was kept stirred at this temperature for 30 min and then cooled to −78° C. A solution of ((1S*,2S*)-2-formylcyclopropyl)methyl acetate (6.18 g, 43.5 mmol, prepared as described in WO 2012/154204) in DCM (80 mL) was added dropwise over 45 min, keeping the IT below −70° C. The mixture was stirred at this temperature for 30 min and allowed to warm to rt over 1 h. The solvent was removed in vacuo and the residue was purified by CC (EA-Hept) to afford the title acetate as a clear oil (4.84 g; 37% yield).

¹H NMR (CDCl₃) δ: 5.84 (d, J=9.0 Hz, 1H); 3.97 (m, 2H); 2.07 (s, 3H); 1.61 (m, 1H); 1.33 (m, 1H); 0.92-0.78 (m, 2H).

MS (ESI, m/z): 295.0 [M+H^{+]} for C₈H₁₀O₂Br₂; t_{R}=0.87 min.

### H.ii. ((1S,2S)-2-(bromoethynyl)cyclopropyl)methyl Acetate and ((1R,2R)-2-(bromoethynyl)cyclopropyl)methyl Acetate

To a solution of intermediate H.i (3.94 g; 13.2 mmol) in THF (75 mL) was added TBAF trihydrate (23.2 g; 72.8 mmol). The reaction mixture was heated at 60° C. for 4 h. The reaction mixture was cooled to rt and diluted with Et₂O (150 mL). The org. phase was washed with water (60 mL) and brine (60 mL), dried over MgSO₄ and concentrated to dryness. The residue was purified by CC (EA-Hept) to afford the title compound as a yellow oil (1.76 g, 61% yield). The racemic product was separated by semi-preparative chiral HPLC Method A (Hept-EtOH 9-1; flow rate: 20 mL/min, UV detection at 223 nm), the respective retention times (flow rate: 0.8 mL/min) were 5.9 and 8.7 min. The title enantiomers were obtained as clear oils (0.64 g each).

First-Eluting Enantiomer, (1S,2S)-Configurated:

¹H NMR (CDCl₃) δ: 3.97 (dd, J=6.5, 11.7 Hz, 1H); 3.84 (dd, J=7.5, 11.7 Hz, 1H); 2.06 (s, 3H); 1.50 (m, 1H); 1.25 (m, 1H); 0.97 (m, 1H); 0.76 (m, 1H).

[α]_{D}=+96° (c=1.03; MeOH).

Second-Eluting Enantiomer, (1R,2R)-Configurated:

¹H NMR (CDCl₃) δ: 3.97 (dd, J=6.5, 11.7 Hz, 1H); 3.84 (dd, J=7.5, 11.7 Hz, 1H); 2.06 (s, 3H); 1.50 (m, 1H); 1.25 (m, 1H); 0.97 (m, 1H); 0.76 (m, 1H).

[α]_{D}=−94° (c=1.01; MeOH).

The respective absolute configurations of these compounds have been determined though transformation of the second-eluting enantiomer into the corresponding (S) and (R) α-methoxy-α-trifluoromethylphenylacetyl esters and the subsequent analysis of their NMR spectra as described by Tsuda et al. in Chem. Pharm. Bull. (2003), 51, 448-451.

### Preparation I: (2R)-4-(2-bromo-6-oxo-4,6-dihydro-5H-thieno[2,3-c]pyrrol-5-yl)-2-methyl-2-(methylsulfonyl)-N-(((2RS)-tetrahydro-2H-pyran-2-yl)oxy)butanamide

Starting from the compound of Preparation B (0.988 g; 3.9 mmol) and 3-methyl-2-thiophenecarboxylic acid (0.566 g; 3.9 mmol), and proceeding sequentially as described in Preparation C and Preparation D, steps D.iv (Variant B) and D.v, the title compound (0.085 g) was obtained as a yellow gum.

¹H NMR (d6-DMSO) δ: 11.35 (s, 0.5H); 11.32 (s, 0.5H); 7.47 (s, 0.5H); 7.46 (s, 0.5H); 4.88-4.84 (m, 0.5H); 4.61-4.58 (m, 0.5H); 4.49-4.36 (m, 2H); 4.08-3.92 (m, 1H); 3.59-3.49 (m, 2H); 3.46-3.40 (m, 1H); 3.06 (s, 1.5H); 3.03 (s, 1.5H); 2.65-2.47 (overlapped m, 1H); 2.00-1.91 (m, 1H); 1.68-1.60 (m, 3H); 1.59-1.45 (overlapped m, 3H); 1.56 (s, 1.5H); 1.54 (s, 1.5H).

MS (ESI, m/z): 494.86 [M+H⁺] for C₁₇H₂₃N₂O₆BrS₂; t_{R}=0.74 min.

Alternatively, the title compound can be prepared as described hereafter:

### I.i. Tert-butyl (2R)-2-methyl-2-(methylsulfonyl)-4-(6-oxo-4,6-dihydro-5H-thieno[2,3-c]pyrrol-5-yl)butanoate

A solution of thiophenedicarboxaldehyde (1.06 g; 7.37 mmol) and compound of Preparation B (1.92 g; 7.63 mmol) in DCM (64 mL), cooled to 0° C., was added AcOH (1.8 mL; 31.5 mmol). The reaction proceeded for 4 h. The reaction mixture was concentrated to dryness and the residue was co-evaporated twice with cyclohexane (2×10 mL). The residue was purified by CC (Hept-EA-MeOH) to afford the title compound (2.71 g; 98% yield) as a reddish solid.

¹H NMR (d6-DMSO) δ: 7.96 (d, J=4.7 Hz, 1H); 7.23 (d, J=4.7 Hz, 1H); 4.47-4.36 (m, 2H); 3.62 (m, 1H); 3.50 (m, 1H); 3.11 (s, 3H); 2.53-2.47 (overlapped m, 1H); 2.03 (m, 1H); 1.52 (s, 3H); 1.33 (s, 9H).

MS (ESI, m/z): 373.9 [M+H⁺] for C₁₆H₂₃NO₅S₂; t_{R}=0.76 min.

### I.ii. Tert-butyl (2R)-4-(2-bromo-6-oxo-4,6-dihydro-5H-thieno[2,3-c]pyrrol-5-yl)-2-methyl-2-(methylsulfonyl)butanoate

To a solution of intermediate I.i (2.71 g; 7.26 mmol) in AcOH (14 mL) and water (7 mL) was added dropwise bromine (0.44 mL; 8.55 mmol). The resulting solution was stirred at the same temperature for 1 h. Water (20 mL) was added and the mixture was extracted with EA (2×20 mL). The combined extracts were washed with 10% aq. NaHSO₃ (20 mL) and sat. aq. NaHCO₃ (20 mL). The evaporation residue was purified by CC (Hept-EA-MeOH) to afford the title compound (1.65 g, 50% yield) as a yellow gum.

¹H NMR (d6-DMSO) δ: 7.48 (s, 1H); 4.42 (d, J=6.2 Hz, 2H); 3.59 (m, 1H); 3.50 (m, 1H); 3.10 (s, 3H); 2.52-2.48 (overlapped m, 1H); 2.03 (m, 1H); 1.51 (s, 3H); 1.35 (s, 9H).

MS (ESI, m/z): 453.7 [M+H⁺] for C₁₆H₂₂NO₅BrS₂; t_{R}=0.84 min.

### I.iii. (2R)-4-(2-bromo-6-oxo-4,6-dihydro-5H-thieno[2,3-c]pyrrol-5-yl)-2-methyl-2-(methylsulfonyl)-N-(((2RS)-tetrahydro-2H-pyran-2-yl)oxy)butanamide

Starting from intermediate I.ii (1.60 g; 3.53 mmol) and proceeding sequentially as described in Preparation D, steps D.iv (Variant A, >95% yield) and D.v (80% yield), the title compound (0.497 g) was obtained as a yellow gum.

¹H NMR (d6-DMSO) δ: (mixture of diastereomers) 11.35 (s, 0.5H); 11.32 (s, 0.5H); 7.47 (s, 0.5H); 7.46 (s, 0.5H); 4.88-4.84 (m, 0.5H); 4.61-4.58 (m, 0.5H); 4.49-4.36 (m, 2H); 4.08-3.92 (m, 1H); 3.59-3.49 (m, 2H); 3.46-3.40 (m, 1H); 3.06 (s, 1.5H); 3.03 (s, 1.5H); 2.65-2.47 (overlapped m, 1H); 2.00-1.91 (m, 1H); 1.68-1.60 (m, 3H); 1.59-1.45 (overlapped m, 3H); 1.56 (s, 1.5H); 1.54 (s, 1.5H).

MS (ESI, m/z): 494.86 [M+H⁺] for C₁₇H₂₃N₂O₆BrS₂; t_{R}=0.74 min.

### Preparation J: 1-(bromoethynyl)cyclopropan-1-amine Hydrochloride

### J.i. Tert-butyl (1-formylcyclopropyl)carbamate

To a solution of tert-butyl (1-(hydroxymethyl)cyclopropyl)carbamate (commercial, 15 g; 80.3 mmol) in DCM (235 mL), cooled to −20° C., was slowly added DIPEA (45 mL; 263 mmol). A solution of Pyr.SO₃ (38.75 g; 110 mmol) in DMSO (108 mL, 1.5 mol) was added dropwise over 45 min, keeping the IT below −5° C. The reaction mixture was stirred at this temperature for 3 h. The reaction mixture was partitioned between water (1 L) and DCM (200 mL). The two layers were separated and the aq. layer was extracted once more with DCM (300 mL). The evaporation residue was evaporated further with toluene (2×300 mL) and purified by CC (Hept-EA) to afford the title product (13.18 g, 89% yield) as a white solid.

¹H NMR (d6-DMSO) δ: 8.99 (s, 1H); 7.56 (s, 1H); 1.41-1.34 (overlapped m, 2H); 1.39 (s, 9H); 1.16-1.13 (m, 2H).

### J.ii. Tert-butyl (1-(2,2-dibromovinyl)cyclopropyl)carbamate

Starting from the intermediate J.i (13.1 g; 71.2 mmol) and proceeding as described in Preparation H, step H.i (90% yield), the title compound (21.8 g) was obtained, after purification by CC (Hept-EA), as a white solid.

¹H NMR (d6-DMSO) δ: 7.46 (s, 1H); 6.48 (s, 1H); 1.37 (s, 9H); 0.94-0.97 (m, 2H); 0.89-0.92 (m, 2H).

### J.iii. Tert-butyl (1-(bromoethynyl)cyclopropyl)carbamate

A solution of intermediate J.ii (13.64 g; 40 mmol) in THF (90 mL) cooled at −78° C., was treated with a fresh suspension of tBuOK (24.73 g; 220 mmol) in THF (220 mL) over 1 h. The reaction proceeded for 3 h at −78° C. After warming to −10° C. over 1 h, brine (450 mL) and Et₂O (360 mL) were added. The aq. layer was separated and extracted with Et20 (360 mL). The evaporation residue was purified by CC (Hept-EA) to afford the title compound (8.34 g; 80% yield) as a white solid.

¹H NMR (d6-DMSO) δ: 7.61 (s, 1H); 1.38 (s, 9H); 1.07-1.03 (m, 2H); 0.95-0.91 (m, 2H).

### J.iv. 1-(bromoethynyl)cyclopropan-1-amine Hydrochloride

A solution of intermediate J.iii (8.34 g; 32 mmol) in 4M HCl in dioxane (33 mL; 132 mmol) was stirred for 3 h. After evaporation to dryness, the residue was triturated with Et₂O (50 mL) to give after filtration and drying to a constant weight the title compound (6.24 g, 99% yield) as a white solid.

¹H NMR (d6-DMSO) δ: 8.94 (s, 2H); 1.34-1.27 (m, 2H); 1.27-1.20 (m, 2H).

MS (ESI, m/z): 201.01 [M+MeCN+H⁺] for C₇H₁₀N₂Br; t_{R}=0.24 min.

### Preparation K: (2R)-4-(2-ethynyl-6-oxo-4,6-dihydro-5H-thieno[2,3-c]pyrrol-5-yl)-2-methyl-2-(methylsulfonyl)-N-((2RS)-(tetrahydro-2H-pyran-2-yl)oxy)butanamide

Starting from the compound of Preparation I (0.497 g; 1 mmol) and proceeding sequentially in analogy to Procedure C (76% yield) and Preparation F, step F.ii (>95% yield), the title compound (0.334 g) was obtained as a brownish solid.

¹H NMR (d6-DMSO) δ: (mixture of diastereomers) 11.35 (s, 0.5H); 11.32 (s, 0.5H); 7.50 (s, 0.5H); 7.49 (s, 0.5H); 4.87 (m, 1H); 4.86 (m, 0.5H); 4.61 (m, 0.5H); 4.48-4.37 (m, 2H); 4.03 (m, 0.5H); 3.97 (m, 0.5H); 3.61-3.50 (m, 2H); 3.43 (m, 1H); 3.06 (s, 1.5H); 3.03 (s, 1.5H); 2.64-2.46 (overlapped m, 1H); 1.96 (m, 1H); 1.66-1.60 (m, 2H); 1.59-1.46 (overlapped m, 4H); 1.56 (s, 1.5H); 1.55 (s, 1.5H).

MS (ESI, m/z): 440.95 [M+H⁺] for C₁₉H₂₄N₂O₆S₂; t_{R}=0.72 min.

### Preparation L: ((1R,2R)-2-(bromoethynyl)-1-fluorocyclopropyl)methyl benzoate

### L.i. ((1R*,2R*)-2-(((tert-butyldiphenylsilyl)oxy)methyl)-1-fluorocyclopropyl)methanol

To a solution of ethyl (1R*,2R*)-2-(((tert-butyldiphenylsilyl)oxy)methyl)-1-fluorocyclopropane-1-carboxylate (0.5 g; 1.25 mmol; prepared as described in Sakagami et al., Bioorg. Med. Chem. (2008), 16(8), 4359-4366) in THF (9 mL), cooled to −78° C., was added dropwise LiBH₄ (2M in THF; 2.2 mL; 4.4 mmol). The reaction mixture was allowed to reach rt and stirred at rt for 24 h. MeOH (2 mL) was carefully added, the reaction mixture was stirred for 20 min, concentrated to dryness and partitioned between water (10 mL) and DCM (15 mL). The aq. layer was extracted with DCM (2×10 mL). The combined org. layers were dried over Na₂SO₄ and filtered. After concentration of the filtrate to dryness, the title compound was obtained as a colourless oil (0.429 g; 96% yield).

¹H NMR (CDCl₃) δ: 7.72-7.66 (m, 4H); 7.45-7.36 (m, 6H); 3.89 (ddd, J=1.6, 6.0, 11.0 Hz, 1H); 3.83-3.80 (m, 1H); 3.78-3.70 (m, 2H); 1.74 (t, J=6.4 Hz, 1H); 1.33-1.24 (m, 1H); 1.05 (s, 9H); 0.88-0.79 (m, 2H).

MS (ESI, m/z): 358.95 [M+H⁺] for C₂₁H₂₇O₂FSi; t_{R}=1.01 min.

### L.ii. ((1R*,2R*)-2-(((tert-butyldiphenylsilyl)oxy)methyl)-1-fluorocyclopropyl)methyl benzoate

To a solution of intermediate L.i (5.51 g, 15.4 mmol) in THF (93 mL) was added TEA (6 mL; 43.1 mmol). Benzoyl chloride (3.6 mL; 30.7 mmol) was added dropwise over 2 min at 0° C. The reaction mixture was stirred at 0° C. for 5 h before being poured onto water (75 mL). The aq. layer was extracted with EA (3×50 mL). The combined org. layers were dried over MgSO₄ and concentrated to dryness. The residue was purified by CC (Hept-EA) to afford the title compound as a colourless oil (6.49 g; 91% yield).

¹H NMR (CDCl₃) δ: 8.12-8.09 (m, 2H); 7.70-7.67 (m, 4H); 7.56 (m, 1H); 7.44-7.40 (m, 4H); 7.38-7.35 (m, 4H); 4.62 (m, 1H); 4.51 (ddd, J=1.1, 13.0, 23.8 Hz, 1H); 3.93 (ddd, J=1.5, 5.6, 11.0 Hz, 1H); 3.70 (ddd, J=1.1, 8.4, 10.9 Hz, 1H); 1.46 (m, 1H); 1.30 (m, 1H); 1.02 (s, 7H); 0.97 (m, 1H); 0.91-0.84 (m, 2H).

MS (ESI, m/z): 463.07 [M+H⁺] for C₂₈H₃₁O₃FSi; t_{R}=1.14 min.

### L.iii. ((1R*,2R*)-1-fluoro-2-(hydroxymethyl)cyclopropyl)methyl Benzoate

To a solution of intermediate L.ii (6.49 g; 14 mmol) in THF (26 mL) was added TBAF (1M in THF, 17 mL). The reaction mixture was stirred at rt for 45 min. The reaction mixture was concentrated in vacuo and the residue was purified by CC (DCM-MeOH) to afford the title compound (2.81 g; 89% yield) as a yellow oil.

¹H NMR (CDCl₃) δ: 8.10-8.08 (m, 2H); 7.58 (m, 1H); 7.48-7.45 (m, 2H); 4.64 (n, 1H); 4.55 (m, 1H); 3.97 (ddd, J=1.5, 5.8, 11.8 Hz, 1H); 3.68 (ddd, J=1.4, 8.7, 11.8 Hz, 1H); 1.52 (m, 1H); 1.12-1.04 (m, 2H).

### L.iv. ((1R,2R)-2-(2,2-dibromovinyl)-1-fluorocyclopropyl)methyl Benzoate

Starting from intermediate L.iii (2.77 g; 12.4 mmol) and proceeding successively in analogy to Preparation J, step J.i (84% yield) and Preparation H, step H.i (adding 2 eq. TEA, 77% yield), a mixture of enantiomers (2.71 g) was obtained. After separation by semi-preparative chiral HPLC Method C (Hept-EtOH 3-7; flow rate: 16 mL/min, UV detection at 224 nm), the title enantiomer (first-eluting enantiomer) was obtained as a white solid (1.25 g). The retention time on analytical chiral HPLC (Hept-EtOH 3-7; flow rate: 0.8 mL/min) was 5.3 min.

¹H NMR (d6-DMSO) δ: 8.01-7.99 (m, 2H); 7.69 (m, 1H); 7.58-7.54 (m, 2H); 6.38 (dd, J=1.4, 8.9 Hz, 1H); 4.75-4.57 (m, 2H); 2.09 (m, 1H); 1.55-1.48 (m, 2H).

### L.v. ((1R,2R)-2-(bromoethynyl)-1-fluorocyclopropyl)methyl Benzoate

To a solution of intermediate L.iv (2.05 g, 5.42 mmol) in THF (20 mL) was added TBAF (1M in THF, 22 mL; 21.7 mmol). The mixture was stirred overnight. The reaction mixture was diluted with EA (50 mL) and water (30 mL). The two layers were separated and the org. layer was extracted with EA (3×50 mL). The evaporation residue was purified by CC (Hept-EA) to afford the title compound (1.1 g; 68% yield) as a yellowish oil.

¹H NMR (d6-DMSO) δ: 8.03-7.99 (m, 2H); 7.70 (m, 1H); 7.60-7.55 (m, 2H); 4.67-4.51 (m, 2H); 2.09-2.04 (m, 1H); 1.49-1.37 (m, 2H).

### Preparation M: 1-(bromoethynyl)-N-methylcyclopropan-1-amine Hydrochloride

### Mi. Tert-butyl (1-(((tert-butyldiphenylsilyl)oxy)methyl)cyclopropyl)carbamate

To a solution of tert-butyl (1-(hydroxymethyl)cyclopropyl)carbamate (3.5 g; 18.7 mmol) and imidazole (2.54 g; 37.4 mmol) in DCM (40 mL) was added TBDPSCl (4.11 mL; 18.7 mmol). The reaction mixture was stirred for 4 h. Water (50 mL) and DCM (20 mL) were added. The two layers were separated and the aq. phase was extracted twice with DCM (2×25 mL). The evaporation residue was purified by CC (EA-Hept) to afford the title compound (8.85 g; >95% yield) as a colorless oil.

¹H NMR (d6-DMSO) δ: 7.64-7.60 (m, 4H); 7.49-7.40 (m, 6H); 7.20 (s, 1H); 3.66 (s, 2H); 1.36 (br s, 9H); 1.00 (s, 9H); 0.71-0.65 (m, 2H); 0.64-0.60 (m, 2H).

MS (ESI, m/z): 426.1 [M+H⁺] for C₂₅H₃₅NO₃Si; t_{R}=1.11 min.

### M.ii. Tert-butyl (1-(((tert-butyldiphenylsilyl)oxy)methyl)cyclopropyl)(methyl)carbamate

A suspension of NaH (60% in oil dispersion, 1.33 g; 33.2 mmol) in dry DMF (21 mL) was added dropwise to an ice-chilled solution of intermediate M.i (7.85 g; 18.4 mmol) in dry DMF (13 mL). The reaction mixture was stirred for 30 min then MeI (1.38 mL; 22.1 mmol) was added dropwise. After 3 h stirring at rt, water (200 mL) was added carefully and the resulting suspension was extracted with EA (2×100 mL). The evaporation residue was purified by CC (Hept-EA) to afford the title compound (5.78 g, 71% yield) as a white solid.

MS (ESI, m/z): 440.1 [M+H⁺] for C₂₆H₃₇NO₃Si; t_{R}=1.15 min.

### M iii. 1-(bromoethynyl)-N-methylcyclopropan-1-amine Hydrochloride

Starting from the intermediate M.ii (6.57 g; 14.9 mmol), and proceeding successively in analogy to Preparation L, step L.iii (97% yield), Preparation J, step J.i (91% yield), Preparation H, step H.i (91% yield) and Preparation J, steps J.iii (98% yield) and J.iv (98% yield), the title compound (2.4 g) was obtained, after final trituration in Et20, as a white solid.

¹H NMR (d6-DMSO) δ: 9.73 (s, 2H); 2.65 (s, 3H); 1.46-1.42 (m, 2H); 1.29-1.24 (m, 2H).

MS (ESI, m/z): 173.99 [M+H⁺] for C₆HsNBr; t_{R}=0.35 min.

Preparation N: 3-bromoprop-2-yn-1-yl 3-hydroxyazetidine-1-carboxylate:

### N.i. 3-bromoprop-2-yn-1-yl (2,5-dioxopyrrolidin-1-yl)carbonate

To a solution of 3-bromoprop-2-yn-1-ol (1 g; 7.41 mmol) in MeCN (85 mL) was added TEA (2.1 mL; 14.8 mmol) and DSC (6.0 g; 22.2 mmol). The reaction mixture was stirred for 30 min. The reaction mixture was diluted with EA (100 mL) and washed with 5% aq. citric acid aq. (3×50 mL). The evaporation residue was purified by CC (Hept-EA) to afford the title product (1.38 g; 67% yield) as a beige solid.

¹H NMR (d6-DMSO) δ: 5.13 (s, 2H); 2.83 (s, 4H).

### N.ii. 3-bromoprop-2-yn-1-yl 3-hydroxyazetidine-1-carboxylate

To a solution of intermediate N.i (1.38 g; 5 mmol) in DCM (65 mL) were added 3-hydroxyazetidine hydrochloride (0.559 g, 5 mmol) and TEA (1.39 mL; 10 mmol). After 45 min stirring, the reaction mixture was diluted in DCM (200 mL) and washed with sat. NaHCO₃ (3×200 mL). The evaporation residue was purified by CC (Hept-EA) to afford the title compound (0.875 g) as a white solid.

¹H NMR (d6-DMSO) δ: 5.35 (m, 1H); 4.26-4.22 (m, 2H); 3.88 (d, J=8.4 Hz, 2H); 2.82 (s, 4H); 1.39 (m, 9H).

### Preparation O: (2R)-4-(5-ethynyl-1-oxoisoindolin-2-yl)-2-methyl-2-(methylsulfonyl)-N-(((2RS)-tetrahydro-2H-pyran-2-yl)oxy)butanamide

### O.i. tert-butyl (2R)-4-(5-bromo-1-oxoisoindolin-2-yl)-2-methyl-2-(methylsulfonyl)butanoate

NaBH(OAc)₃ (1.48 g; 7 mmol) was added to a solution of the compound of Preparation B (1.6 g; 6.37 mmol), methyl 4-bromo-2-formylbenzoate (1.53 g, 6.23 mmol) and AcOH (0.365 mL, 6.37 mmol) in DCM (30 mL).The reaction mixture was stirred for 5 h. DCM (50 mL) and sat. NaHCO₃ (100 mL) were added. The two layers were separated. The evaporation residue was purified by CC (Hept-EA) to afford the title compound (1.47 g; 52% yield) as a white solid.

¹H NMR (d6-DMSO) δ: 7.90 (d, J=1.0 Hz, 1H); 7.68 (dd, J=1.7, 8.1 Hz, 1H); 7.61 (m, 1H); 4.49 (m, 2H); 3.71-3.65 (m, 1H); 3.55 (m, 1H); 3.12 (s, 3H); 2.51 (overlapped m, 1H); 2.06 (m, 1H); 1.54 (s, 3H); 1.34 (s, 9H).

MS (ESI, m/z): 447.92 [M+H⁺] for C₁₈H₂₄NO₅BrS; t_{R}=0.85 min.

### O.ii. tert-butyl (2R)-4-(5-iodo-1-oxoisoindolin-2-yl)-2-methyl-2-(methylsulfonyl)butanoate

To a solution of intermediate O.i (1.37 g; 3.07 mmol) in dioxane (7 mL) were added trans-N,N-dimethylcyclohexane-1,2-diamine (0.194 mL; 1.23 mmol), NaI (0.920 g; 6.14 mmol) and CuI (0.117 g; 0.614 mmol). The reaction mixture was then heated for 3 h at 125° C. After cooling. the solvent was evaporated and the residue was taken up in water (200 mL) and EA (250 mL). The aq. layer was extracted with EA (2×200 mL). The evaporation residue was purified by CC (Hept-EA) to afford the title product (1.17 g; 74% yield) as a yellow solid.

¹H NMR (d6-DMSO) δ: 8.07 (s, 1H); 7.85 (dd, J=1.2, 7.9 Hz, 1H); 7.46 (d, J=7.9 Hz, 1H); 4.46 (m, 2H); 3.70-3.64 (m, 1H); 3.54 (m, 1H); 3.12 (s, 3H); 2.51 (overlapped m, 1H); 2.05 (m, 1H); 1.53 (s, 3H); 1.34 (s, 9H).

MS (ESI, m/z): 493.91 [M+H⁺] for C₁₈H₂₄NO₅IS; t_{R}=0.87 min.

### O.iii. (2R)-4-(5-iodo-1-oxoisoindolin-2-yl)-2-methyl-2-(methylsulfonyl)butanoic Acid

To an ice-chilled solution of intermediate O.ii (1.11 g; 2.26 mmol) in DCM (6.5 mL) was added Et₃SiH (0.4 mL, 2.5 mmol) and TFA (5 mL; 65.3 mmol). After stirring for 4 h, the reaction mixture was cooled to 0° C. and Et₂O (12 mL) was added dropwise. The solid was filtered off and scarcely washed with Et₂O to afford, after drying to a constant weight, the title compound (0.93 g; 94% yield) as a yellow solid.

¹H NMR (d6-DMSO) δ: 13.71 (m, 1H); 8.05 (s, 1H); 7.85 (dd, J=1.1, 7.9 Hz, 1H); 7.45 (d, J=7.9 Hz, 1H); 4.45 (m, 2H); 3.73-3.68 (m, 1H); 3.56 (m, 1H); 3.12 (s, 3H); 2.57-2.53 (m, 1H); 2.04 (m, 1H); 1.55 (s, 3H).

MS (ESI, m/z): 437.87 [M+H⁺] for C₁₄H₁₆NO₅IS; t_{R}=0.68 min.

### O.iv. (2R)-4-(5-ethynyl-1-oxoisoindolin-2-yl)-2-methyl-2-(methylsulfonyl)-N-(((2RS)-tetrahydro-2H-pyran-2-yl)oxy)butanamide

Starting from the intermediate O.iii (0.83 g; 1.91 mmol), and proceeding successively in analogy to Preparation D, step D.v (98% yield), Procedure C (94% yield), and Preparation F, step F.ii (79% yield), the title compound (0.587 g) was obtained, after purification by CC (Hept-EA), as a beige foam.

MS (ESI, m/z): 435.01 [M+H⁺] for C₂₁H₂₆N₂O₆S; t_{R}=0.73 min.

### Preparation P: 3-(bromoethynyl)azetidine Hydrochloride

### P.i. Tert-butyl 3-(bromoethynyl)azetidine-1-carboxylate

To a solution of tert-butyl 3-ethynylazetidine-1-carboxylate (3 g; 16.6 mmol, prepared as described in WO 2014/165075) and NBS (3.55 g; 19.9 mmol) in acetone (66 mL) was added AgNO3 (0.3 g; 1.77 mmol). The mixture was stirred for 2 h. After filtration over Celite and evaporation of solvent under reduced pressure, the residue was purified by CC (Hex-TBME) to afford the title compound (4 g, 93% yield) as a yellow oil.

¹H NMR (CDCl₃) δ: 4.14 (m, 2H); 3.96 (dd, J=6.3 Hz, 8.4 Hz, 2H); 3.34 (m, 1H); 1.46 (s, 9H).

### P.ii. 3-(bromoethynyl)azetidine Hydrochloride

Starting from the intermediate P.i (0.670 g, 2.7 mmol) and proceeding in analogy to Preparation J, step J.iv, the title compound was obtained, after trituration in Et₂O, as an off-white solid (0.49 g; 97% yield).

¹H NMR (CDCl₃) δ: 9.44-9.10 (m, 2H); 4.15-4.06 (m, 2H); 3.96-3.87 (m, 2H); 3.74 (m, 1H).

MS (ESI, m/z): 162.0 [M+H⁺] for C₅H₆NBr; t_{R}=0.23 min.

### Preparation Q: (2R)-4-(5-bromo-1-oxoisoindolin-2-yl)-2-methyl-2-(methylsulfonyl)-N-(((2RS)-tetrahydro-2H-pyran-2-yl)oxy)butanamide

Starting from the intermediate O.i (0.98 g; 2.2 mmol) and proceeding in analogy to Preparation O, step O.iii (92% yield) and Preparation D, step D.v (57% yield), the title compound (0.54 g) was obtained after purification by CC (Hept-EA) as a yellowish foam.

MS (ESI, m/z): 489.15 [M+H⁺] for C₁₉H₂₅N₂O₆BrS; t_{R}=0.75 min.

### Preparation R: (1-(bromoethynyl)cyclopropyl)methanol

Starting from the compound of Preparation G (3 g; 7.26 mmol) and proceeding in analogy to Preparation L, step L.iii (88% yield), the title compound (1.12 g) was obtained after purification by CC (Hept-EA) as a colorless oil.

¹H NMR (d6-DMSO) δ: 4.90 (t, J=6.0 Hz, 1H); 3.32 (d, J=6.0 Hz, 2H); 0.80-0.77 (m, 2H); 0.76-0.72 (m, 2H).

### Preparation S: 3-fluoro-1-(4-iodobenzyl)azetidine

A mixture of 4-iodobenzyl bromide (0.250 g, 0.842 mmol), 3-fluoroazetidine hydrochloride (0.297 g; 2.53 mmol) and K₂CO₃ (0.465 g; 3.37 mmol) in DMF (7.5 mL) was stirred for 7 h. The reaction mixture was poured into ice water (5 mL) and extracted with DCM (2×10 mL). The evaporation residue afforded the title compound (0.20 g; 83% yield) as a yellow liquid.

¹H NMR (CDCl₃) δ: 7.66-7.63 (m, 2H); 7.04-7.02 (m, 2H); 5.13 (m, 1H); 3.69-3.62 (m, 2H); 3.61 (s, 2H); 3.23-3.14 (m, 2H).

MS (ESI, m/z): 291.89 [M+H⁺] for C₁₀H₁₁NFI; t_{R}=0.54 min.

### Preparation T: ((1R,2R)-2-(bromoethynyl)-1-fluorocyclopropyl)methyl Di-Tert-Butyl Phosphate

### T.i. ((1R,2R)-2-(bromoethynyl)-1-fluorocyclopropyl)methanol

To a solution of the compound of Preparation L (1.400 g; 4.71 mmol) in MeOH (23 mL) was added K₂CO₃ (1.302 g; 9.42 mmol). The suspension was stirred for 45 min. DCM (230 mL) was added and the mixture was washed with 10% NaHSO₄ solution (60 mL). The aq. layer was extracted with DCM-MeOH (9-1, 2×200 mL). The evaporation residue was purified by CC (Hept-EA) to afford the title compound (0.625 g, 69% yield) as a yellowish oil.

¹H NMR (d6-DMSO) δ: 5.17 (t, J=6.0 Hz, 1H); 3.68-3.52 (m, 2H); 1.72 (m, 1H); 1.28-1.15 (m, 2H).

### T.ii. ((1R,2R)-2-(bromoethynyl)-1-fluorocyclopropyl)methyl Di-Tert-Butyl Phosphate

To a solution of intermediate T.i (0.258 g; 1.34 mmol) in THF (2 mL) cooled to 0° C. was added portionwise NaH (60% in mineral oil, 0.0802 g; 2.01 mmol). The reaction mixture was stirred at 0° C. for 30 min and di-tert-butyl phosphorochloridate (0.428 g, 1.87 mmol) was added dropwise, keeping the IT below 8° C. The reaction proceeded overnight. EA (20 mL) and H₂O (20 mL) were added. The layers were separated and the aq. layer was extracted with EA (20 mL). The evaporation residue was purified by CC (Hept-EA) to afford the title compound (0.386 g; 75% yield) as a colorless oil.

¹H NMR (CDCl₃) δ: 4.18-3.99 (m, 2H); 1.92 (m, 1H); 1.41 (s, 18H); 1.39-1.30 (m, 2H).

MS (ESI, m/z): 385.00 [M+H⁺] for C₁₄H₂₃O₄BrFP; t_{R}=0.91 min.

### Preparation U: 3-(bromoethynyl)-1-(2-((tert-butyldimethylsilyl)oxy)ethyl)azetidine

To a solution of the compound of Preparation P (1.5 g; 7.63 mmol) in DCM (76 mL) were added (tert-butyldimethylsilyloxy)acetaldehyde (4.44 mL; 21 mmol) and NaBH(OAc)₃ (9.5 g; 45 mmol). The reaction mixture was stirred for 3 h. Sat. aq. NaHCO₃ (80 mL) and DCM (10 mL) were added. The aq. layer was extracted with DCM (2×70 mL). The evaporation residue was purified by CC (Hept-EA) to afford the title compound as a yellow oil (2.29 g; 94% yield).

¹H NMR (d6-DMSO) δ: 3.52 (t, J=5.7 Hz, 2H); 3.46 (t, J=7.3 Hz, 2H); 3.23-3.16 (m, 1H); 2.98 (t, J=7.8 Hz, 2H); 2.44 (t, J=5.7 Hz, 2H); 0.86 (s, 9H); 0.03 (s, 6H).

MS (ESI, m/z): 317.99 [M+H⁺] for C₁₃H₂₄NOBrSi; t_{R}=0.74 min.

### Preparation V: (3aR,5S,6aS)-5-(bromoethynyl)-2,2-dimethyltetrahydro-4H-cyclopenta[d][1,3]dioxole

### V.i. (3aR,5s,6aS)-5-(bromoethynyl)-2,2-dimethyltetrahydro-4H-cyclopenta[d][1,3]dioxole

Starting from (3aR,5S,6aS)-5-(2,2-dibromovinyl)-2,2-dimethyltetrahydro-4H-cyclopenta[d][1,3]dioxole (2.43 g; 6.32 mmol; prepared as described in WO 2013/170030) and proceeding in analogy to Preparation J, step J.iii, the title compound was obtained as a yellow oil (1.80 g; 99% yield).

¹H NMR (CDCl₃) δ: 4.63-4.60 (m, 2H); 2.93-2.85 (m, 1H); 2.17-2.12 (m, 2H); 1.60-1.51 (overlapped m, 2H); 1.41 (s, 3H); 1.26 (s, 3H).

### Vii. (3aR,5S,6aS)-5-(bromoethynyl)-2,2-dimethyltetrahydro-4H-cyclopenta[d][1,3]dioxole

A solution of intermediate V.i (1.01 g; 4.13 mmol) in 1M HCl (20 mL) and THF (20 mL) was stirred at 50° C. for 1 h. After cooling, EA (50 mL) was added and the two layers were separated. The aq. layer was saturated with solid NaCl and extracted with EA (2×25 mL). The evaporation residue was purified by CC (Hept-EA-MeOH) to afford the title compound (0.658 g, 78% yield) as a white solid.

¹H NMR (d6-DMSO) δ: 4.49-4.47 (m, 2H); 3.94-3.89 (m, 2H); 2.97 (m, 1H); 1.88-1.83 (m, 2H); 1.71-1.66 (m, 2H).

### Preparation W: (2R)-1-((1S,2S)-2-(bromoethynyl)cyclopropyl)ethane-1,2-diol

### W.i. ((1S,2S)-2-((4R)-2,2-dimethyl-1,3-dioxolan-4-yl)cyclopropyl)methanol

To a mixture of trimethylsulfonium iodide (1.32 g; 6.0 mmol) and NaH (60% dispersion in oil; 0.24 g; 6.0 mmol) was added DMSO (6 mL) dropwise. The reaction mixture was stirred 1 h, and a solution of tert-butyl (R,E)-3-(2,2-dimethyl-1,3-dioxolan-4-yl)acrylate (as prepared in Sugano et al., Chemistry—A European Journal (2012), 18(31), 9682-9690; 1.14 g; 5.0 mmol) in THF (6 mL) was added dropwise. The reaction mixture was stirred at rt overnight. Brine (30 mL) was added dropwise and the resulting mixture was extracted with Et₂O (3×30 mL). The combined extracts were washed with brine (4×10 mL), dried over Na₂SO₄, filtered and carefully evaporated to dryness. The crude (1.2 g) was taken up in THF (20 mL) and the solution was cooled to 0° C. LiAlH₄ (0.38 g; 10 mmol) was added. The reaction proceeded at 0° C. for 1 h. Water (0.3 mL), 1M NaOH (0.3 mL) and water (1 mL) were added. The resulting mixture was filtered through a pad of celite and washed with THF (50 mL). The filtrate was concentrated to dryness and the residue was purified by CC (Hept-EA) to afford the title compound as a yellowish oil (0.78 g; 75% yield).

¹H NMR (CDCl₃) δ: 4.09 (dd, J=6.0, 8.0 Hz, 1H); 3.69 (m, 1H); 3.61 (td, J=6.0, 7.6 Hz, 1H); 3.53-3.45 (m, 2H); 1.43 (s, 3H); 1.34 (s, 3H); 1.05 (m, 1H); 0.89 (m, 1H); 0.68 (dt, J=5.0, 8.5 Hz, 1H); 0.58 (dt, J=5.1, 8.4 Hz, 1H).

### W.ii. (1S,2S)-2-((4R)-2,2-dimethyl-1,3-dioxolan-4-yl)cyclopropane-1-carbaldehyde

Starting from intermediate W.i (2.52 g; 14.6 mmol) and proceeding as described in Preparation J, step J.i, the title compound was obtained, after purification by CC (EA-Hept), as a yellowish oil (1.78 g; 71% yield).

¹H NMR (CDCl₃) δ: 9.12 (d, J=5.1 Hz, 1H); 4.11 (dd, J=6.1, 8.2 Hz, 1H); 3.81 (q, J=6.6 Hz, 1H); 3.70 (dd, J=6.8, 8.2 Hz, 1H); 1.84-1.90 (m, 1H); 1.67-1.73 (m, 1H); 1.43 (s, 3H); 1.34 (s, 3H); 1.21-1.27 (m, 2H).

### W.iii. (4R)-4-((1S,2S)-2-ethynylcyclopropyl)-2,2-dimethyl-1,3-dioxolane

A suspension of intermediate W.ii (2.32 g; 13.6 mmol) and K₂CO₃ (3.767 g: 27.3 mmol) in MeOH (12.5 mL) was treated dropwise with dimethyl (1-diazo-2-oxopropyl)phosphonate (2.880 g; 15 mmol). The reaction mixture was stirred at rt for 2 h. The solvent was evaporated and the residue was dissolved in DCM (20 mL) and water (15 mL). The aq. layer was extracted once with DCM (15 mL). The evaporation residue afford the title compound as a yellow oil (1.74 g; 77% yield).

¹H NMR (CDCl₃) δ: 4.13 (dd, J=6.0, 8.1 Hz, 1H); 3.77 (m, 1H); 3.68 (m, 1H); 1.83 (d, J=2.1 Hz, 1H); 1.44 (s, 3H); 1.34 (m, 1H); 1.35 (s, 3H); 1.27-1.22 (m, 1H); 1.02-0.92 (m, 2H).

### W.iv. (4R)-4-((1S,2S)-2-(bromoethynyl)cyclopropyl)-2,2-dimethyl-1,3-dioxolane

Starting from intermediate W.iii (1.74 g; 10.5 mmol) and proceeding in analogy to Preparation P, step P.i, the title compound was obtained, after purification by CC (DCM-Hept), as a yellowish oil (0.7 g; 27% yield).

¹H NMR (CDCl₃) δ: 4.13 (dd, J=6.0, 8.1 Hz, 1H); 3.78 (dd, J=7.0, 8.1 Hz, 1H); 3.68 (m, 1H); 1.83 (d, J=2.1 Hz, 1H); 1.44 (s, 3H); 1.35 (s, 3H); 1.34 (overlapped m, 1H); 1.25 (m, 1H); 1.00 (m, 1H); 0.95 (m, 1H).

### W.v. (2R)-1-((1S,2S)-2-(bromoethynyl)cyclopropyl)ethane-1,2-diol

A solution of intermediate W.iv (0.663 g; 2.7 mmol) in 1M aq. HCl (3.26 mL) and THF (0.652 mL) was stirred at 50° C. EA (15 mL) was added to the mixture and the two layers were separated. The aq. layer was saturated with NaCl and extracted again with EA (2×15 mL). The evaporation residue to afford a yellow oil (0.492 g; 90% yield).

¹H NMR (CDCl₃) δ: 3.80 (dd, J=3.2, 11.2 Hz, 1H); 3.63 (dd, J=7.4, 11.1 Hz, 1H); 3.30 (td, J=3.2, 7.0 Hz, 1H); 1.33-1.26 (m, 3H); 0.94-0.88 (m, 2H).

### Preparation X: 3-(bromoethynyl)-1-(2-fluoroethyl)azetidine

To a suspension of the compound of Preparation P (0.6 g, 1.19 mmol) in MeOH (11 mL) were added sequentially TEA (0.48 mL; 3.46 mmol) and 1-iodo-2-fluoroethane (0.46 mL; 5.25 mmol). The reaction mixture was stirred at 60° C. overnight. The reaction mixture was cooled at rt and concentrated under reduced pressure. The residue was purified by CC (Hex-EA) to afford the title product as a colourless oil (0.24 g, 98% yield).

¹H NMR (CDCl₃) δ: 4.41 (m, 1H); 4.32 (m, 1H); 3.52-3.47 (m, 2H); 3.23 (quint, J=7.4 Hz, 1H); 3.05-3.01 (m, 2H); 2.67 (m, 1H); 2.61 (m, 1H).

MS (ESI, m/z): 206.0 [M+H⁺] for C₁₇H₉NBrF; t_{R}=0.27 min.

### Preparation Y: 3-(bromoethynyl)-1-isopropylazetidine

Starting from the compound of Preparation P (0.65 g; 3.31 mmol) and acetone (0.067 mL; 9.1 mmol) and proceeding in analogy to Preparation U, the title compound was obtained, after purification by CC (DCM-MeOH gradient), as a colourless oil (0.67 g; >95% yield).

¹H NMR (CDCl₃) δ: 3.41-3.37 (m, 2H); 3.10 (quint, J=7.4 Hz, 1H); 2.91-2.85 (m, 2H); 2.22 (hep, J=6.1 Hz, 1H); 0.81 (d, J=6.2 Hz, 6H).

MS (ESI, m/z): 204.0 [M+H⁺] for C₈H₁₂NBr; t_{R}=0.37 min.

### Preparation Z: 1-(3-(bromoethynyl)azetidin-1-yl)-2-methylpropan-2-ol

To a solution of the compound of Preparation P (0.73 g; 3.72 mmol) in EtOH (58 mL) was added 1,2-epoxy-2-methylpropane (0.51 mL; 5.57 mmol) and TEA (1.55 mL; 11.1 mmol) at rt. The mixture was heated at 60° C. for 3 h. The reaction mixture was evaporated to dryness and the residue was purified by CC (DCM-MeOH gradient) to afford the title compound as a brown oil (0.57 g; 67% yield).

¹H NMR (d6-DMSO) δ: 4.04 (s, 1H); 3.55-3.50 (m, 2H); 3.21 (quint, J=7.6 Hz, 1H); 3.02-2.99 (m, 2H); 2.26 (s, 2H); 1.01 (s, 6H).

### Preparation AA: 3-(4-iodophenyl)-1-(oxetan-3-yl)azetidine

### AA.i. 3-(4-iodophenyl)azetidine Hydrochloride

Starting from tert-butyl 3-(4-bromophenyl)azetidine-1-carboxylate (1.32 g; 4.23 mmol) and proceeding successively in analogy to Preparation O. step O.ii (70% yield) and Preparation J, step J.iv (96% yield), the title compound was obtained, after trituration in Et₂O, as an off-white solid (0.27 g).

MS (ESI, m/z): 259.9 [M+H⁺] for C₉H₁₀NI; t_{R}=0.54 min.

### AA. ii. 3-(4-iodophenyl)-1-(oxetan-3-yl)azetidine

Starting from intermediate AA.i (0.54 g; 1.83 mmol) and 3-oxetanone (0.198 g; 2.74 mmol) proceeding in analogy to Preparation U, the title compound was obtained after purification by CC (DCM-MeOH), as an off-white solid (0.369 g; 64% yield).

¹H NMR (d6-DMSO) δ: 7.68 (d, J=8.4 Hz, 2H); 7.20 (d, J=8.3 Hz, 2H); 4.55-4.58 (m, 2H); 4.38 (dd, J=5.4 Hz, 6.2 Hz, 2H); 3.74 (m, 1H); 3.56-3.66 (m, 3H); 3.13-3.16 (m, 2H).

MS (ESI, m/z): 259.9 [M+H⁺] for C₁₂H₁₄NOI; t_{R}=0.56 min.

### Preparation AB: 3-(bromoethynyl)-1-(tetrahydro-2H-pyran-4-yl)azetidine

Starting from the compound of Preparation P (0.60 g; 3.0 mmol) and tetrahydro-4H-pyran-4-one (0.067 mL; 9.1 mmol) and proceeding in analogy to Preparation U, the title compound was obtained, without purification, as a beige solid (0.67 g; >95% yield).

¹H NMR (d6-DMSO) δ: 3.80-3.75 (m, 2H); 3.45-3.40 (m, 2H); 3.29-3.23 (m, 2H); 3.17 (m, 1H); 2.95-2.89 (m, 2H); 2.19 (m, 1H); 1.58-1.51 (m, 2H); 1.12-1.04 (m, 2H).

MS (ESI, m/z): 245.99 [M+H⁺] for C₁₀H₁₄NOBr; t_{R}=0.35 min.

### Preparation AC: 3-(bromoethynyl)-1-(oxetan-3-ylmethyl)azetidine

Starting from the compound of Preparation P (0.5 g; 2.57 mmol) and oxetane-3-carbaldehyde (0.264 g; 2.91 mmol) and proceeding in analogy to Preparation U, the title compound was obtained, without purification, as a yellow oil (0.60 g; >95% yield).

¹H NMR (d6-DMSO) δ: 4.56 (dd, J=5.9, 7.8 Hz, 2H); 4.22-4.19 (m, 2H); 3.42-3.39 (m, 2H); 3.18 (quint, J=7.4 Hz, 1H); 2.94-2.91 (m, 2H); 2.87 (m, 1H); 2.61 (d, J=7.5 Hz, 2H).

### Preparation AD: 3-(4-iodophenyl)-1-(oxetan-3-ylmethyl)azetidine

Starting from intermediate AA.i (0.536 g; 1.82 mmol) and oxetane-3-carbaldehyde (0.191 g; 2.11 mmol) and proceeding in analogy to Preparation U, the title compound was obtained, after purification by CC (DCM-MeOH), as a colourless oil (0.469 g; 79% yield).

¹H NMR (d6-DMSO) δ: 7.68-7.64 (m, 2H); 7.16-7.13 (m, 2H); 4.59 (dd, J=5.8, 7.8 Hz, 2H); 4.26 (t, J=6.0 Hz, 2H); 3.58-3.51 (m, 3H); 3.07-3.00 (m, 2H); 2.94 (m, 1H); 2.73-2.68 (m, 2H).

MS (ESI, m/z): 329.9 [M+CH₃CN+H⁺] for C₁₃H₁₆NOI; t_{R}=0.56 min.

### Preparation AE: 1-(((1S,2S)-2-(bromoethynyl)cyclopropyl)methyl)-3-fluoroazetidine

### AE.i. ((1S,2S)-2-(bromoethynyl)cyclopropyl)methanol

Starting from the (S,S)-configurated intermediate H.ii (1.09 g; 5.0 mmol) and proceeding in analogy to Preparation T, step T.i, the title compound was obtained without purification as a yellowish oil (0.95 g; >95% yield).

¹H NMR (CDCl₃) δ: 4.56 (dd, J=5.9, 7.8 Hz, 2H); 4.22-4.19 (m, 2H); 3.42-3.39 (m, 2H); 3.18 (quint, J=7.4 Hz, 1H); 2.94-2.91 (m, 2H); 2.87 (m, 1H); 2.61 (d, J=7.5 Hz, 2H).

### AE.ii. ((1S,2S)-2-(bromoethynyl)cyclopropyl)methyl 4-methylbenzenesulfonate

To a solution of intermediate AE.i (0.950 g; 5.0 mmol) in DCM (9 mL), cooled at 0° C., were added TEA (1.4 mL; 10 mmol) and TsCl (1.09 g; 5.64 mmol). The solution was stirred overnight at rt. The reaction mixture was diluted with DCM (10 mL) and washed with sat. aq. NaHCO₃ (10 mL). The aq. layer was extracted with DCM (10 mL). The evaporation residue was purified by CC (Hept-EA) to afford the title compound as a colourless oil (1.43 g; 86% yield).

¹H NMR (CDCl₃) δ: 7.80-7.78 (m, 2H); 7.36-7.34 (m, 2H); 3.94 (dd, J=6.8, 10.9 Hz, 1H); 3.85 (dd, J=7.5, 10.9 Hz, 1H); 2.46 (s, 3H); 1.46 (m, 1H); 1.20 (ddd, J=4.4, 5.4, 8.9 Hz, 1H); 0.97 (dt, J=5.2, 8.6 Hz, 1H); 0.74 (dt, J=5.5, 8.8 Hz, 1H).

### AE.iii. 1-(((1S,2S)-2-(bromoethynyl)cyclopropyl)methyl)-3-fluoroazetidine

Starting from intermediate AE.ii (1.43 g; 4.35 mmol) and proceeding in analogy to Preparation S, the title compound was obtained, after purification by CC (DCM-MeOH), as a yellowish oil (0.7 g; 70% yield).

¹H NMR (CDCl₃) δ: 5.14 (m, 1H); 3.80-3.72 (m, 2H); 3.17 (ddd, J=5.2, 8.0, 23.5 Hz, 2H); 2.46-2.36 (m, 2H); 1.19 (m, 1H); 1.09 (m, 1H); 0.91 (dt, J=4.9, 8.6 Hz, 1H); 0.65 (m, 1H).

MS (ESI, m/z): 232.0 [M+H⁺] for C₉H₁₁NBrF; t_{R}=0.43 min.

### Preparation AF: (1r,3r)-3-(3-(bromoethynyl)azetidin-1-yl)cyclobutyl Acetate and (1s,3s)-3-(3-(bromoethynyl)azetidin-1-yl)cyclobutyl Acetate

Starting from the compound of Preparation P (0.5 g; 2.56 mmol) and 3-oxocyclobutyl acetate (0.366 g, 2.85 mmol) and proceeding in analogy to Preparation U, the title compounds were obtained respectively after CC (Hept-EA). The first eluting isomer, the (1r,3r)-isomer (trans) was obtained as a yellowish oil (0.217 g; 31% yield). The second eluting isomer, the (1s,3s)-isomer (cis) was obtained as a yellowish oil that crystallized on standing (0.21 g; 30% yield).

(1r,3r)-isomer:

¹H NMR (CDCl₃) δ: 5.09 (m, 1H); 3.66-3.57 (m, 2H); 3.24 (m, 1H); 3.19 (m, 1H); 3.09-3.01 (m, 2H); 2.29-2.22 (m, 2H); 2.14-2.06 (m, 2H); 2.02 (s, 3H).

MS (ESI, m/z): 271.9 [M+H⁺] for C₁₁H₁₄NO₂Br; t_{R}=0.45 min.

(1s,3s)-isomer:

¹H NMR (CDCl₃) δ: 5.09 (m, 1H); 4.71 (m, 1H); 3.62-3.54 (m, 2H); 3.27 (m, 1H); 3.19-3.13 (m, 2H); 2.88 (m, 1H); 2.53-2.48 (m, 2H); 2.02 (s, 3H); 1.99-1.93 (m, 2H).

MS (ESI, m/z): 271.9 [M+H⁺] for C₁₁H₁₄NO₂Br; t_{R}=0.44 min.

### Preparation AG: 3-((tert-butyldimethylsilyl)oxy)propyl 3-(bromoethynyl)azetidine-1-carboxylate

### AG.i. 3-((tert-butyldimethylsilyl)oxy)propyl (2,5-dioxopyrrolidin-1-yl)carbonate

Starting 3-((tert-butyldimethylsilyl)oxy)propan-1-ol (commercial, 0.4 g; 2.04 mmol) and proceeding as described in Preparation N, step N.i, the title compound was obtained, after purification by CC (Hept-EA), as a colourless oil (0.637 g; 94% yield).

¹H NMR (d6-DMSO) δ: 4.41 (t, J=6.3 Hz, 2H); 3.68 (t, J=6.0 Hz, 2H); 2.81 (s, 4H); 1.88 (quint, J=6.2 Hz, 2H); 0.87 (s, 9H); 0.05 (s, 6H).

### AG.ii. 3-((tert-butyldimethylsilyl)oxy)propyl 3-(bromoethynyl)azetidine-1-carboxylate

Starting from intermediate AG.i (0.635 g; 2 mmol) and the compound of Preparation P (0.393 g; 2 mmol) and proceeding in analogy to Preparation N, step N.ii, the title compound was obtained, after purification by CC (Hept-EA), as a colourless oil (0.580 g; 77% yield).

¹H NMR (d6-DMSO) δ: 4.13 (m, 2H); 4.02 (t, J=6.1 Hz, 2H); 3.77-3.81 (m, 2H); 3.64 (t, J=6.1 Hz, 2H); 3.51 (m, 1H); 1.72 (quint, J=6.2 Hz, 2H); 0.87 (s, 9H); 0.04 (s, 6H).

MS (ESI, m/z): 377.9 [M+H⁺] for C₁₅H₂₆NO₃BrSi; t_{R}=1.02 min.

### Preparation AH: ((2R,3R)-3-(bromoethynyl)-1-methylazetidin-2-yl)methanol

### AH.i. Tert-butyl (2R)—N-allyl-N-(3-(benzyloxy)-2-hydroxypropyl)glycinate

A flask was charged with (R)-benzyl glycidyl ether (40.0 g; 244 mmol) and allylamine (183 mL; 2436 mmol). Water (1 mL) was added to the mixture and the reaction was warmed to 55° C. and stirred overnight. After removal of the solvent, the crude product (54 g; 100% yield) was obtained as a yellowish oil. The latter (54.0 g; 244 mmol) was taken up in THF (500 mL) and tert-butyl bromoacetate (54 mL; 366 mmol) and TEA (68 mL; 488 mmol) were added. The mixture was allowed to stir at rt for 1 h. The reaction mixture was partitioned between water (500 mL) and Et₂O (500 mL). The two phases were separated and the aq. phase was extracted twice with Et₂O (500 mL). The evaporation residue was purified by CC (Hept-EA) to give the title compound as a colourless oil (68 g, 83% yield).

¹H NMR (CDCl₃) δ: 7.38-7.29 (m, 5H); 5.89-5.78 (m, 1H); 5.23-5.14 (m, 2H); 4.61-4.57 (m, 2H); 3.91-3.84 (m, 1H); 3.73 (s, 1H); 3.51 (m, 2H); 3.40-3.33 (m, 1H); 3.29-3.22 (m, 3H); 2.84-2.79 (m, 1H); 2.65-2.56 (m, 1H); 1.51-1.46 (m, 9H).

MS (ESI, m/z): 336.1 [M+H⁺] for C₁₉H₃₀NO₄; t_{R}=0.71 min.

### AH.ii. Tert-butyl (2R)—N-allyl-N-(3-(benzyloxy)-2-chloropropyl)glycinate

To a solution of AH.i. (68.0 g; 203 mmol) in DCM (500 mL) was added thionyl chloride (30.3 mL; 416 mmol) and the mixture was heated to reflux for 1 h. The mixture was partitioned between DCM (100 mL) and sat. NaHCO₃ (500 mL). The two phases were separated and the aq. phase was extracted with DCM (500 mL). The evaporation residue was taken up in DMF (500 mL) and the mixture was heated to 65° C. for 2 days. The mixture was diluted with water (500 mL) and Et₂O (500 mL) and the phases were separated. The aq. phase was extracted twice with Et₂O (500 mL). The evaporation residue was purified by CC (Hept-EA) to give the title compound as a colourless oil (60 g; 84% yield).

¹H NMR (CDCl₃) δ: 7.30-7.41 (m, 5H); 5.73-5.89 (m, 1H); 5.11-5.26 (m, 2H); 4.57-4.68 (m, 2H); 4.10 (m, 1H); 3.77-3.82 (m, 1H); 3.72 (m, 1H); 3.34-3.40 (m, 4H); 3.09-3.17 (m, 1H); 2.90-3.04 (m, 1H); 1.47-1.51 (m, 9H).

MS (ESI, m/z): 353.9 [M+H⁺] for C₁₉H₂₉NO₃Cl; t_{R}=0.84 min.

### AH.iii. Tert-butyl (2S,3R)-1-allyl-3-((benzyloxy)methyl)azetidine-2-carboxylate and Tert-Butyl (2R,3R)-1-allyl-3-((benzyloxy)methyl)azetidine-2-carboxylate

A solution of intermediate AH.ii (58.7 g, 166 mmol) in THF (600 mL)/HMPA (60 mL) was cooled to −78° C. and a solution of LiHMDS (1M in THF, 250 mL, 250 mmol) was added slowly. The mixture was allowed to warm to 0° C. over 3 h. The reaction was quenched by addition of sat. NH₄Cl. The aq. phase was extracted twice with EA (500 mL). The evaporation residue was purified by CC (Hept-EA) to afford both title diastereomers as colourless oils ((2S,3R): 35.3 g, 67% yield; (2R,3R): 7.8 g, 15% yield).

(2S,3R)-isomer:

¹H NMR (CDCl₃) δ: 7.39-7.29 (m, 5H); 5.92-5.77 (m, 1H); 5.23-5.15 (m, 1H); 5.14-5.06 (m, 1H); 4.55-4.51 (m, 2H); 3.85-3.79 (m, 1H); 3.76-3.69 (m, 1H); 3.67-3.62 (m, 1H); 3.29-3.25 (m, 1H); 3.18-3.12 (m, 2H); 2.97 (t, J=7.4 Hz, 1H); 2.89-2.82 (m, 1H); 1.46-1.41 (m, 9H).

MS (ESI, m/z): 318.1 [M+H⁺] for C₁₉H₂₈NO₃; t_{R}=0.72 min.

(2R,3R)-isomer:

¹H NMR (CDCl₃) δ: 7.44-7.34 (m, 5H); 5.92-5.79 (m, 1H); 5.26-5.17 (m, 1H); 5.15-5.07 (m, 1H); 4.60-4.54 (m, 2H); 3.62-3.51 (m, 2H); 3.50-3.43 (m, 2H); 3.35-3.26 (m, 1H); 3.08 (m, 1H); 2.90-2.82 (m, 2H); 1.52-1.44 (m, 9H).

MS (ESI, m/z): 318.1 [M+H+] for C₁₉H₂₈NO₃; t_{R}=0.72 min.

### AH.iv. ((2R,3R)-1-allyl-3-((benzyloxy)methyl)azetidin-2-yl)methanol

A solution of (2R,3R)-configurated intermediate AH.iii (7.8 g; 24.6 mmol) in THF (50 mL) was cooled to 0° C. and a solution of LiA1H₄ (2M in THF, 25 mL; 50 mmol) was slowly added. The mixture was stirred at 0° C. for 1 h and then warmed to rt. After 2 h the reaction was quenched by careful addition of 1M aq. NaOH (20 mL) and the resulting slurry was stirred for 1 h. The solid was filtered off and the filtrate was concentrated to dryness. The crude product (6 g; >95% yield) was used without further purification in the following step.

¹H NMR (CDCl₃) δ: 7.42-7.29 (m, 5H); 5.84-5.72 (m, 1H); 5.24-5.18 (m, 1H); 5.15-5.08 (m, 1H); 4.59-4.50 (m, 2H); 3.62-3.56 (m, 1H); 3.55-3.40 (m, 4H); 3.24-3.12 (m, 2H); 3.12-3.03 (m, 1H); 3.03-2.91 (m, 1H); 2.79-2.66 (m, 2H).

MS (ESI, m/z): 248.1 [M+H⁺] for C₁₅H₂₂NO₂; t_{R}=0.57 min.

### AH.v. (2R,3R)-1-allyl-3-((benzyloxy)methyl)-2-(((tert-butyldipheny-lsilyl)oxy)methyl) azetidine

Starting from intermediate AH.iv (6.0 g; 24.3 mmol) and proceeding in analogy to Preparation M, step M.i, the title compound was obtained, after purification by CC (Hept-EA), as a colourless oil (11.7 g; >95% yield).

¹H NMR (CDCl₃) δ: 7.73-7.67 (m, 5H); 7.49-7.29 (m, 10H); 5.85-5.70 (m, 1H); 5.21-5.12 (m, 1H); 5.10-5.01 (m, 1H); 4.58-4.44 (m, 2H); 3.86-3.77 (m, 1H); 3.74-3.67 (m, 1H); 3.60-3.44 (m, 3H); 3.39-3.27 (m, 1H); 3.19-3.11 (m, 1H); 3.06-2.94 (m, 1H); 2.78-2.65 (m, 1H); 2.61-2.47 (m, 1H); 1.12-1.03 (m, 9H).

MS (ESI, m/z): 486.2 [M+H⁺] for C₃₁H₃₉NO₂Si; t_{R}=0.94 min.

### AH.vi. Tert-butyl (2R,3R)-3-((benzyloxy)methyl)-2-(((tert-butyldiphenylsilyl)oxy)methyl)azetidine-1-carboxylate

To a solution of intermediate AH.v. (11.7 g; 24.1 mmol) in DCM-EtOH mixture (1:2; 200 mL) was added N-methyl barbituric acid (5.64 g; 36.1 mmol) and Pd(PPh₃)₄ (1.39 g; 1.2 mmol). The reaction mixture was stirred at rt for 30 min. The solvent was removed in vacuo and the residue was dissolved in DCM (200 mL) and Boc₂O (7.88 g; 36.1 mmol) was added and the mixture was stirred for 18 h. The solvent was removed in vacuo and the evaporation residue was directly subjected to CC (Hept-EA) to afford the title compound as a colourless oil (13.5 g; >95% yield).

¹H NMR (CDCl₃) δ: 7.65-7.73 (m, 4H); 7.31-7.48 (m, 11H); 4.55 (s, 2H); 3.96-4.06 (m, 2H); 3.72-3.81 (m, 1H); 3.59-3.69 (m, 3H); 2.86-2.97 (m, 1H); 2.72-2.78 (m, 1H); 1.40 (s, 9H); 1.08 (s, 9H).

MS (ESI, m/z): 546.1 [M+H⁺] for C₃₃H₄₄NO₄Si; t_{R}=1.16 min.

### AH.vii. Tert-butyl (2R,3R)-2-(((tert-butyldiphenylsilyl)oxy)methyl)-3-(hydroxymethyl)azetidine-1-carboxylate

To a solution of intermediate AH.vi (14 g, 25.7 mmol) in MeOH (200 mL) was added Pd/C (10 wt %; 2 g). The mixture was stirred under a hydrogen atmosphere. After 5 days, the suspension was filtered and the filtrate was concentrated. Purification by CC (Hept-EA) provided the title compound as a colourless oil (4.45 g; 38% yield) along with reisolated starting material.

¹H NMR (CDCl₃) δ: 7.69 (m, 4H); 7.50-7.36 (m, 6H); 4.11-4.00 (m, 1H); 3.97-3.90 (m, 2H); 3.88-3.84 (m, 1H); 3.84-3.76 (m, 2H); 3.67-3.57 (m, 1H); 2.82-2.69 (m, 1H); 1.39 (s, 9H); 1.14-1.06 (m, 9H).

MS (ESI, m/z): 456.14 [M+H⁺] for C₂₆H₃₇NO₄Si; t_{R}=1.04 min.

### AH.viii. Tert-butyl (2R,3R)-2-(((tert-butyldiphenylsilyl)oxy)methyl)-3-formylazetidine-1-carboxylate

Starting from intermediate AH.vii (1.2 g; 2.63 mmol) and proceeding in analogy to Preparation J, step J.i, the title compound was obtained, after purification by CC (Hept-EA), as a colourless oil (0.96 g; 81% yield).

¹H NMR (CDCl₃) δ: 9.77 (s, 1H); 7.67-7.60 (m, 4H); 7.50-7.41 (m, 6H); 4.36 (m, 1H); 4.08-3.82 (m, 3H); 3.75 (m, 1H); 3.50 (m, 1H); 1.28 (m, 9H) 1.02 (s, 9H).

MS (ESI, m/z): 454.15 [M+H⁺] for C₂₆H₃₅NO₄Si; t_{R}=1.07 min.

### AH.ix. Tert-butyl (2R,3R)-3-(bromoethynyl)-2-(hydroxymethyl)azetidine-1-carboxylate

Starting from intermediate AH.viii (1.62 g; 3.57 mmol) and proceeding successively in analogy to Preparation H, steps H.i (77% yield) and H.ii (88% yield), the title compound was obtained, after purification by CC (Hept-EA), as a colourless oil (0.701 g).

¹H NMR (d6-DMSO) δ: 4.95 (t, J=5.6 Hz, 1H); 4.04 (m, 1H); 3.88 (m, 1H); 3.65-3.59 (m, 2H); 3.52 (m, 1H); 3.26 (m, 1H); 1.38 (s, 9H).

### AH.x. ((2R,3R)-3-(bromoethynyl)azetidin-2-yl)methanol

To a solution of intermediate AH.ix (0.264 g; 0.91 mmol) in MeCN (1.1 mL) was added H₂SO₄ (0.27 mL, 4.89 mmol) in H₂O (2.4 mL). The reaction was stirred at 60° C. for 1 h. The solution was cooled to rt, then 15% aq. NaOH was added until pH=7. The mixture was concentrated to dryness. The residue was triturated in a DCM-MeOH mixture (9-1; 50 mL) for 40 min. After filtration, the residue was dissolved in DCM-MeOH (9-1; 10 mL), dried over Na₂SO₄, filtered and evaporated to afford the title compound as a white solid (0.121 g; 70% yield).

¹H NMR (d6-DMSO) δ: 4.01 (m, 1H); 3.67-3.56 (m, 2H); 3.50 (d, J=4.7 Hz, 2H); 3.44-3.24 (overlapped m, 3H).

MS (ESI, m/z): 190.02 [M+H⁺] for C₆H₈NOSBr; t_{R}=0.22 min.

### AH.xi. ((2R,3R)-3-(bromoethynyl)-1-methylazetidin-2-yl)methanol

To a suspension of intermediate AH.x (0.119 g; 0.63 mmol) in DCM (8 mL) were added 37% aq. formaldehyde (0.054 mL; 1.88 mmol) and NaBH(OAc)₃ (0.698 g; 3.19 mmol). The reaction mixture was stirred at rt for 40 min. Sat. aq. NaHCO₃ (5 mL) and DCM (10 mL) were slowly added. The aq. layer was extracted with DCM (2×15 mL). The evaporation residue afforded the title compound as a yellowish oil (0.098 g; 77% yield).

MS (ESI, m/z): 206.03 [M+H⁺] for C₇H₁₀NOSBr; t_{R}=0.24 min.

### Preparation AI: ((2R,4RS)-4-(bromoethynyl)-1-methylpyrrolidin-2-yl)methanol

### AI.i. Tert-butyl (2R,4R)-2-(((tert-butyldiphenylsilyl)oxy)methyl)-4-((methylsulfonyl)oxy)pyrrolidine-1-carboxylate

To a stirred solution of tert-butyl (2R,4R)-2-(((tert-butyldiphenylsilyl)oxy)methyl)-4-hydroxypyrrolidine-1-carboxylate (prepared as described in WO 2014/078609; 2 g; 4.39 mmol) and TEA (1.22 mL; 8.78 mmol) in DCM (22 mL) at 0° C. was added MsCl (0.35 mL; 4.52 mmol). The reaction mixture was allowed to reach rt over 30 min. Sat. aq. NaHCO₃ (15 mL) was added and the phases were separated. The aq. layer was extracted once with DCM (10 mL). The evaporation residue afforded the crude title compound as a yellow gum (2.37 g; >95% yield).

MS (ESI, m/z): 534.2.0 [M+H⁺] for C₂₇H₃₉NO₆SSi; t_{R}=1.08 min.

### AI.ii. Tert-butyl (2R,4RS)-2-(((tert-butyldiphenylsilyl)oxy)methyl)-4-iodopyrrolidine-1-carboxylate

To a solution of intermediate AI.i (2.37 g, 4.39 mmol) in 2-butanone (17 mL) was added NaI (2 g, 13.4 mmol). The reaction mixture was stirred at 80° C. for 26 h. The reaction mixture was cooled to rt, diluted with water (30 mL) and EA (20 mL). The aq. layer was extracted once with EA (20 mL). The evaporation residue was purified by CC (Hept-EA) to afford the title compound as a colorless oil (2.04 g; 81% yield).

MS (ESI, m/z): 566.1 [M+H⁺] for C₂₆H₃₆NO₃IS; t_{R}=1.16 min.

### AI.iii. Tert-butyl (2R,4RS)-2-(((tert-butyldiphenylsil)oxy)methyl)-4-((trimethylsilyl)ethynyl)pyrrolidine-1-carboxylate

EtMgBr (1M in THF; 2.65 mL; 2.65 mmol) was added dropwise to a solution of TMS-acetylene (0.38 mL; 2.65 mmol) dissolved in THF (2.7 mL). The mixture was stirred 15 min at rt then 1 h at 50° C. In a separated flask, FeBr₂ (0.06 g, 0.27 mmol) and intermediate AI.ii (1 g; 1.77 mmol) were dissolved in THF (4.5 mL) and NMP (2 mL). The previous warmed Grignard reagent solution was added drop wise over 8 min. The resulting dark mixture was stirred at rt for 3 h. EA (20 mL) and water (15 mL). The two layers were separated. The evaporation residue was purified by CC (Hept-EA) to afford the title compound as an orange gum (0.79 g; 84% yield).

MS (ESI, m/z): 536.2 [M+H⁺] for C₃₁H₄₅NO₃Si₂; t_{R}=1.20 min.

### AI.iv. Tert-butyl (2R,4RS)-2-(((tert-butyldiphenylsilyl)oxy)methyl)-4-ethynylpyrrolidine-1-carboxylate

A solution of intermediate AI.iii (0.71 g; 1.32 mmol) in MeOH (4.5 mL) was treated by K₂CO₃ (0.24 g, 1.72 mmol). The mixture was stirred at rt for 1 h. The reaction was diluted in DCM (50 mL) and water (15 mL). The two layers were separated then the aq. layer was extracted with DCM-MeOH mixture (9-1; 20 mL). The evaporation residue afforded the crude title compound as a yellow oil (0.56 g; 91% yield).

MS (ESI, m/z): 464.2 [M+H⁺] for C₂₈H₃₇NO₃Si; t_{R}=1.13 min.

### AI.v. ((2R,4RS)-4-(bromoethynyl)-1-methylpyrrolidin-2-yl)methanol

Starting from intermediate AI.iv (0.5 g; 1.08 mmol), and proceeding successively in analogy to Preparation P. step P.i (81% yield) and then to Preparation J, step J.iv and Preparation AH, step AH.xi (75% yield over the 2 steps), the title compound was obtained, after purification by CC (DCM-MeOH), as a yellowish oil (0.125 g).

¹H NMR (d6-DMSO) δ: 4.44 (m, 1H); 3.36 (m, 1H); 3.22 (m, 1H); 3.12 (dd, J=6.9, 8.3 Hz, 1H); 2.85 (m, 1H); 2.36 (m, 1H); 2.26 (s, 3H); 2.16 (dd, J=8.6, 10.0 Hz, 1H); 1.91 (m, 1H); 1.83 (m, 1H).

MS (ESI, m/z): 218.0 [M+H⁺] for CsH₁₂NOBr; t_{R}=0.31 min.

### Preparation AJ: (R)-3-(bromoethynyl)-1-(tetrahydrofuran-3-yl)azetidine AND (S)-3-(bromoethynyl)-1-(tetrahydrofuran-3-yl)azetidine

### AJ.i. (RS)-3-(bromoethynyl)-1-(tetrahydrofuran-3-yl)azetidine

Starting from the compound of Preparation P (1.5 g; 7.63 mmol) and dihydrofuran-3(2H)-one (0.738 g; 8.4 mmol) and proceeding in analogy to Preparation U, the title compound was obtained, after purification by CC (Hept-EA), as a white solid (0.98 g; 56% yield).

¹H NMR (d6-DMSO) δ: 3.68-3.60 (m, 2H); 3.50 (dd, J=5.2, 8.9 Hz, 1H); 3.43-3.40 (m, 2H); 3.37 (dd, J=2.5, 8.9 Hz, 1H); 3.17 (quint, J=7.3 Hz, 1H); 2.97-2.90 (m, 3H); 1.73 (m, 1H); 1.55 (m, 1H).

MS (ESI, m/z): 231.9 [M+H⁺] for C₉H₁₂NOBr; t_{R}=0.27 min.

### AJ.ii. (R)-3-(bromoethynyl)-1-(tetrahydrofuran-3-yl)azetidine and (S)-3-(bromoethynyl)-1-(tetrahydrofuran-3-yl)azetidine

Intermediate AJ.i (1.22 g) was separated by semi-preparative chiral HPLC Method D (CO₂-EtOH 9-1+0.1% DEA; flow rate: 160 mL/min; UV detection at 213 nm); the respective retention times were 1.6 and 1.9 min. The first-eluting compound, “intermediate AJ.1”, was obtained as a beige solid (0.448 g). The second-eluting compound, “intermediate AJ.2”., was obtained as a beige solid (0.553 g). The absolute stereochemistry of intermediates AJ.1 and AJ.2 has not been assigned.

First-Eluting Compound (“Intermediate AJ 1”):

¹H NMR (d6-DMSO) δ: 3.68-3.60 (m, 2H); 3.50 (dd, J=5.2, 8.9 Hz, 1H); 3.43-3.40 (m, 2H); 3.37 (dd, J=2.5, 8.9 Hz, 1H); 3.17 (quint, J=7.3 Hz, 1H); 2.97-2.90 (m, 3H); 1.73 (m, 1H); 1.55 (m, 1H).

MS (ESI, m/z): 231.9 [M+H⁺] for C₉H₁₂NOBr; t_{R}=0.27 min.

Analytical chiral HPLC Method E (Hept-EtOH+0.05% DEA, flow rate 0.8 mL/min, detection at 222 nm): t_{R}=5.82 min (e.e. >99%).

Second-Eluting Compound (“Intermediate AJ.2”)

¹H NMR (d6-DMSO) δ: 3.68-3.60 (m, 2H); 3.50 (dd, J=5.2, 8.9 Hz, 1H); 3.43-3.40 (m, 2H); 3.37 (dd, J=2.5, 8.9 Hz, 1H); 3.17 (quint, J=7.3 Hz, 1H); 2.97-2.90 (m, 3H); 1.73 (m, 1H); 1.55 (m, 1H).

MS (ESI, m/z): 231.9 [M+H⁺] for C₉H₁₂NOBr; t_{R}=0.27 min.

Analytical chiral HPLC Method E (Hept-EtOH+0.05% DEA, flow rate 0.8 mL/min, detection at 222 nm): t_{R}=7.36 min (e.e. =90%).

### Preparation AK: 3-((3-(bromoethynyl)azetidin-1-yl)methyl)oxetan-3-ol

### AK.i. 3-hydroxyoxetane-3-carbaldehyde

Into a solution of 3-vinyloxetan-3-ol (0.6 g; 5.99 mmol) in DCM (6.5 mL) and MeOH (53.5 mL), cooled to −78° C., was bubbled an ozone stream. After 1 h, polymer-bound PPh₃ (3 mmol/g; 6 g) was added and the reaction mixture was allowed to warm to rt. The mixture was vigorously stirred overnight. The resine was filtered off and the filtrate was concentrated to dryness to afford the title compound as a colourless oil (0.64 g; >95% yield).

¹H NMR (CDCl₃) δ: 10.11 (s, 1H); 4.91-4.94 (m. 4H).

### AK.ii. 3-((3-(bromoethynyl)azetidin-1-yl)methyl)oxetan-3-ol

Starting from the compound of Preparation P (0.55 g; 2.8 mmol) and dihydrofuran-3(2H)-one (0.372 g; 3.64 mmol) and proceeding in analogy to Preparation U, the title compound was obtained, after purification by CC (DCM-MeOH containing 1% aq. NH₄OH), as a white solid (0.37 g; 55% yield).

¹H NMR (d6-DMSO) δ: 5.50 (s, 1H); 4.31-4.33 (m, 2H); 4.27-4.29 (m, 2H); 3.51-3.55 (m, 2H); 3.23 (m, 1H); 3.05-3.10 (m, 2H); 2.62 (s, 2H).

MS (ESI, m/z): 245.9 [M+H⁺] for C₉H₁₂NO₂Br; t_{R}=0.27 min.

### Preparation AL: ((2R,4R)-4-(bromoethynyl)-1-methylazetidin-2-yl)methanol

### AL.i. Tert-butyl (2R*,4R*)-2-((benzoyloxy)methyl)-4-(hydroxymethyl)azetidine-1-carboxylate

To an ice-chilled solution of tert-butyl (2R*,4R*)-2,4-bis(hydroxymethyl)azetidine-1-carboxylate (prepared as described in Evans et al., J. Med. Chem. (2008), 51, 948-956; 4.7 g; 21.8 mmol) in DCM (150 mL) were added DMAP (0.13 g; 1.09 mmol) and TEA (9.1 mL; 65.3 mmol). Benzoyl chloride (2.3 mL; 19.6 mmol) was added dropwise and the reaction was stirred at 0° C. After 40 min., the reaction mixture was partitioned between water (200 mL) and DCM (200 mL). The two layers were separated and the aq. layer was extracted with DCM (3×200 mL). The evaporation residue was purified by CC (Hept-AcOEt-MeOH) to afford the title compound as a yellow oil (3.3 g; 48% yield).

¹H NMR (d6-DMSO) δ: 8.02-8.00 (m, 2H); 7.69 (t, J=7.4 Hz, 1H); 7.57 (t, J=7.7 Hz, 2H); 4.80 (m, 1H); 4.69 (m, 1H); 4.39-4.27 (m, 2H); 4.18 (m, 1H); 3.68 (m, 1H); 3.52 (m, 1H); 2.27 (m, 1H); 2.16 (m, 1H); 1.31 (m, 9H).

MS (ESI, m/z): 322.1 [M+H⁺] for C₁₇H₂₃NO₅; t_{R}=0.80 min.

### AL.ii. Tert-butyl (2R,4R)-2-((benzoyloxy)methyl)-4-(hydroxymethyl)azetidine-1-carboxylate and Tert-butyl (2S,4S)-2-((benzoyloxy)methyl)-4-(hydroxymethyl)azetidine-1-carboxylate

Intermediate AL.i (5.7 g) was separated by semi-preparative chiral HPLC Method F (CO₂-iPrOH 85-15; flow rate: 160 mL/min; UV detection at 230 nm); the respective retention times were 3.3 and 4.2 min. The first-eluting compound, the (2R,4R)-isomer, was obtained as a beige solid (2.45 g). The second-eluting compound, the (2S,4S)-isomer, was obtained as a beige solid (2.53 g). The absolute stereochemistry of the (2S,4S)-isomer was unambiguously assigned by its transformation into (2S,4S)-2,4-bis(((tert-butyldiphenylsilyl)oxy)methyl)azetidine proceeding successively in analogy to Preparation T, step T.i, Preparation M, step M.i and Preparation D, step D.iv. The specific rotation of the obtained compound, [α]_{D}=+3.1 (c 1.02, CHCl₃), matched the one reported in Shi et al., Tetrahedron: Asymmetry (1999), 10, 1673-1679.

First-Eluting Compound: (2R,4R)-Isomer:

¹H NMR (d6-DMSO) δ: 8.02-8.00 (m, 2H); 7.69 (t, J=7.4 Hz, 1H); 7.57 (t, J=7.7 Hz, 2H); 4.80 (m, 1H); 4.69 (m, 1H); 4.39-4.27 (m, 2H); 4.18 (m, 1H); 3.68 (m, 1H); 3.52 (m, 1H); 2.27 (m, 1H); 2.16 (m, 1H); 1.31 (m, 9H).

Analytical chiral HPLC Method E (Hept-iPrOH 85-15, flow rate 4 mL/min, detection at 210 nm): t_{R}=2.56 min (e.e. >99%).

Second-Eluting Compound: (2S,4S)-Isomer:

¹H NMR (d6-DMSO) δ: 8.02-8.00 (m, 2H); 7.69 (t, J=7.4 Hz, 1H); 7.57 (t, J=7.7 Hz, 2H); 4.80 (m, 1H); 4.69 (m, 1H); 4.39-4.27 (m, 2H); 4.18 (m, 1H); 3.68 (m, 1H); 3.52 (m, 1H); 2.27 (m, 1H); 2.16 (m, 1H); 1.31 (m, 9H).

MS (ESI, m/z): 231.9 [M+H⁺] for C₉H₁₂NOBr; t_{R}=0.27 min.

Analytical chiral HPLC Method E (Hept-iPrOH 85-15, flow rate 0.8 mL/min, detection at 210 nm): t_{R}=3.09 min (e.e. >99%).

### AL.iii. ((2R,4R)-4-(bromoethynyl)-1-methylazetidin-2-yl)methanol

Starting from the (2R,4R)-isomer of intermediate AL.ii (0.55 g; 2.8 mmol) and proceeding successively in analogy to Preparation J, step J.i (>95% yield), Preparation J, steps J.i and J.ii (46 and 87% yield respectively), Preparation T, step T.i (96% yield), Preparation AH, steps AH.x and AH.xi (>95 and 48% yield respectively), the title compound was obtained, after purification by CC (DCM-MeOH), as a yellowish oil (0.1 g).

¹H NMR (d6-DMSO) δ: 4.52 (m, 1H); 4.11 (m, 1H); 3.40-3.36 (m, 2H); 3.22 (m, 1H); 2.22 (s, 3H); 2.14 (m, 1H); 1.92 (m, 1H).

MS (ESI, m/z): 205.9 [M+H⁺] for C₇H₁₀NOBr; t_{R}=0.64 min.

### Preparation AM: (2S,3S)-3-(bromoethynyl)-2-(fluoromethyl)-1-methylazetidine

### AM.i. Tert-butyl (2S,3S)-2-(((tert-butyldiphenylsilyl)oxy)methyl)-3-formylazetidine-1-carboxylate

Starting from (S)-benzyl glycidyl ether (40 g; 244 mmol) and proceeding successively in analogy to Preparation AH, steps AH.i to AH.viii, the title compound was obtained as a colourless oil (2.71 g).

¹H NMR (CDCl₃) δ: 7.73-7.65 (m, 4H); 7.48-7.31 (m, 11H); 4.55 (s, 2H); 4.06-3.96 (m, 2H); 3.77 (m, 1H); 3.64 (m, 3H); 2.91 (m, 1H); 2.75 (m, 1H); 1.40 (s, 9H); 1.08 (s, 9H).

MS (ESI, m/z): 546.1 [M+H⁺] for C₃₃H₄₄NO₄Si; t_{R}=1.16 min.

### AM.ii. Tert-butyl (2S,3S)-3-(bromoethynyl)-2-(((methylsulfonyl)oxy)methyl)azetidine-1-carboxylate

Starting from intermediate AM.i (2.61 g; 5.75 mmol) and proceeding successively in analogy to Preparation W, step W.iii, Preparation P, step P.i, Preparation L, step L.iii and Preparation AI, step AI.i, the title compound was obtained, after purification by CC (Hept-EA), as a colourless oil (1.32 g).

¹H NMR (CDCl₃) δ: 4.43-4.34 (m, 3H); 3.93 (t, J=8.2 Hz, 1H); 3.68 (t, J=6.9 Hz, 1H); 3.36 (overlapped m, 1H); 3.24 (s, 3H); 1.39 (s, 9H).

MS (ESI, m/z): 369.9 [M+H⁺] for C₁₂H₁₈NO₅BrS; t_{R}=0.85 min.

### AM.iii. Tert-butyl (2S,3S)-3-ethynyl-2-(fluoromethyl)azetidine-1-carboxylate

A solution of intermediate AM.iii (1.28 g; 3.48 mmol) in a 1M solution of TBAF in THF (24 mL; 24 mmol) was refluxed for 3 h. After cooling, the solution was partitioned between EA (100 mL) and water (30 mL). The org. layer was further washed with brine. The evaporation residue was purified by CC (Hept-EA) to afford the title compound as a colourless oil (0.34 g, 47% yield).

MS (ESI, m/z): 310.0 [M+H⁺] for C₁₇H₂₄NO₃F; t_{R}=0.92 min.

### AM.iv. (2S,3S)-3-(bromoethynyl)-2-(fluoromethyl)-1-methylazetidine

Starting from intermediate AM.iii (0.285 g; 1.34 mmol) and proceeding successively in analogy to Preparation P, step P.i (94% yield) and Preparation AH, steps AH.x and AH.xi (58% yield over 2 steps), the title compound was obtained as a brown oil (0.154 g).

¹H NMR (d6-DMSO) δ: 4.45 (d, J=4.4 Hz, 1H); 4.36 (d, J=4.4 Hz, 1H); 4.13-4.01 (m, 2H); 3.51-3.46 (m, 2H); 3.30 (overlapped m, 1H).

### REFERENCE EXAMPLES

### Reference Example 1: (2RS)—N-hydroxy-4-(2-((4-(1-hydroxy-2-methylpropan-2-yl)phenyl)ethynyl)-6-oxo-4,6-dihydro-5H-thieno[2,3-c]pyrrol-5-yl)-2-methyl-2-(methylsulfonyl)butanamide

Starting from the compound of Preparation D (0.073 g; 0.148 mmol) and the compound of Preparation E (0.29 g; 0.166 mmol) and proceeding successively in analogy to Procedure A (49% yield) and Procedure B (42% yield), the title compound was obtained, after precipitation in water and trituration in DCM, as an off-white solid (0.015 g).

¹H NMR (d6-DMSO) δ:10.94 (s, 1H); 9.15 (s, 1H); 7.53-7.50 (m, 3H); 7.46-7.43 (m, 2H); 4.73 (t, J=5.3 Hz, 1H); 4.51-4.41 (m, 2H); 3.56 (m, 1H); 3.48-3.40 (overlapped m, 1H); 3.43 (d, J=5.4 Hz, 2H); 3.07 (s, 3H); 2.58 (m, 1H); 1.96 (m, 1H); 1.54 (s, 3H); 1.23 (s, 6H).

MS (ESI, m/z): 505.00 [M+H⁺] for C₂₄H₂₈N₂O₆S₂; t_{R}=0.73 min.

### Reference Example 2: (2RS)—N-hydroxy-4-(2-((1-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)butanamide

### RE2.i. (2RS)-4-(2-((1-(((tert-butyldiphenylsilyl)oxy)methyl)cyclopropyl)buta-1,3-diyn-1-yl)-6-oxo-4,6-dihydro-5H-thieno[2,3-c]pyrrol-5-yl)-2-methyl-2-(methylsulfonyl)-N-((2RS)-(tetrahydro-2H-pyran-2-yl)oxy)butanamide

Starting from the compound of Preparation F (0.096 g; 0.23 mmol) and the compound of Preparation G (0.103 g; 0.25 mmol), and proceeding in analogy to Procedure D, the title compound (0.082 g, 47% yield) was obtained, after purification by CC (Hept-EA-MeOH) as a yellow gum.

MS (ESI, m/z): 773.90 [M+H⁺] for C₄₁H₄₈N₂O₇S₂Si; t_{R}=1.12 min.

### RE2.ii. (2RS)-4-(2-((1-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)-N-((2RS)-(tetrahydro-2H-pyran-2-yl)oxy)butanamide

To a solution of intermediate RE2.i (0.082 g; 0.11 mmol) in THF (0.4 mL) was added TBAF (1M in THF, 0.28 mL). The reaction mixture was stirred for 4 h. The reaction mixture was concentrated in vacuo and the residue was purified by CC (DCM-MeOH gradient) to afford the title compound (0.072 g, quant.) as a yellow gum.

MS (ESI, m/z): 535.0 [M+H⁺] for C₂₅H₃₀N₂O₇S₂; t_{R}=0.76 min.

### RE2.iii. (2RS)—N-hydroxy-4-(2-((1-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)butanamide

Starting from intermediate RE2.ii (0.056 g; 0.106 mmol, crude) and proceeding in analogy to Procedure B, the title compound (0.011 g; 24% yield) was obtained after purification by CC (DCM-MeOH gradient), as an off-white solid.

¹H NMR (d6-DMSO) δ: 10.92 (s, 1H); 9.15 (s, 1H); 7.57 (s, 1H); 5.06 (t, J=6.1 Hz, 1H); 4.43 (s, 2H); 3.54 (m, 1H); 3.47-3.37 (overlapped m, 1H); 3.39 (d, J=6.1 Hz, 2H); 3.05 (s, 3H); 2.61-2.47 (overlapped m, 1H); 1.94 (m, 1H); 1.52 (s, 3H); 0.99-0.95 (m, 2H); 0.94-0.90 (m, 2H).

MS (ESI, m/z): 450.91 [M+H⁺] for C₂₀H₂₂N₂O₆S₂; t_{R}=0.65 min.

### EXAMPLES OF COMPOUNDS ACCORDING TO THE INVENTION

### Example 1: (2R)—N-hydroxy-4-(2-(((1R,2R)-2-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)butanamide

Starting from the compound of Preparation F (0.18 g; 0.41 mmol) and the (1R,2R)-configurated compound of Preparation H (0.11 g; 0.5 mmol) and proceeding in analogy to Procedure D (37% yield) and Procedure B (76% yield), the intermediate racemate (0.051 g) was obtained as a white solid. The latter was separated by semi-preparative chiral HPLC Method B (Hept-(EtOH+1% TFA) 1-9; flow rate: 20 mL/min, UV detection at 327 nm), the respective retention times (flow rate: 1 mL/min) were 5.1 and 9.8 min. The title enantiomer was identified as the second-eluting enantiomer and was obtained (0.019 g) as a brownish solid.

¹H NMR (d6-DMSO) δ: 10.92 (br. s, 1H); 9.12 (br. s, 1H); 7.56 (s, 1H); 4.72 (t, J=5.6 Hz, 1H); 4.43 (s, 2H); 3.54 (m, 1H); 3.39-3.47 (m, 2H); 3.26 (m, 1H); 3.05 (s, 3H); 2.60-2.44 (overlapped m, 1H); 1.94 (m, 1H); 1.59-1.43 (overlapped m, 2H); 1.52 (s, 3H); 0.97 (m, 1H); 0.92 (m, 1H).

MS (ESI, m/z): 450.91 [M+H⁺] for C₂₀H₂₂N₂O₆S₂; t_{R}=0.65 min.

### Example 2: (2R)-4-(2-(2-fluoro-4-methoxyphenyl)-6-oxo-4,6-dihydro-5H-thieno[2,3-c]pyrrol-5-yl)-N-hydroxy-2-methyl-2-(methylsulfonyl)butanamide

Starting from the compound of Preparation I (0.085 g; 0.17 mmol) and 2-fluoro-4-methoxyphenylboronic acid (0.031 g; 0.18 mmol) and proceeding in analogy to Procedure E (94% yield) and Procedure B (20% yield), the title compound (0.015 g) was obtained after purification by CC (DCM-MeOH gradient) as a yellowish solid.

¹H NMR (d6-DMSO) δ: 10.95 (s, 1H); 9.17 (s, 1H); 7.79-7.73 (m, 1H); 7.57 (s, 1H); 7.03 (dd, J=2.5, 13.4 Hz, 1H); 6.91 (dd, J=2.5, 8.8 Hz, 1H); 4.51-4.42 (m, 2H); 3.83 (s, 3H); 3.61-3.54 (m, 1H); 3.46-3.39 (m, 1H); 3.07 (s, 3H); 2.66-2.46 (overlapped m, 1H); 2.00-1.93 (m, 1H); 1.54 (s, 3H).

MS (ESI, m/z): 456.95 [M+H⁺] for C₁₉H₂₁N₂O₆FS₂; t_{R}=0.72 min.

### Example 3: (2R)-4-(2-((1-aminocyclopropyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide

Starting from the compound of Preparation K (0.092 g; 0.2 mmol) and the compound of Preparation J (0.047 g; 0.24 mmol) and proceeding in analogy to Procedure D (74% yield) and Procedure B (42% yield), the title compound (0.028 g) was obtained after purification by CC (DCM-MeOH) as a yellowish solid.

¹H NMR (d6-DMSO) δ: 10.92 (s, 1H); 9.15 (s, 1H); 7.57 (s, 1H); 4.43 (s, 2H); 3.54 (m, 1H); 3.43 (m, 1H); 3.05 (s, 3H); 2.57 (ddd, J=6.6, 9.5, 13.2 Hz, 1H); 1.94 (m, 1H); 1.52 (s, 3H); 1.01-0.98 (m, 2H); 0.91-0.87 (m, 2H).

MS (ESI, m/z): 476.99 [M+H⁺] for C₁₉H₂₁N₃O₅S₂; t_{R}=0.50 min.

### Example 4: (2R)-4-(2-(((1R,2R)-2-fluoro-2-(hydroxymethyl)cyclopropyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide

Starting from the compound of Preparation K (0.25 g; 0.57 mmol) and the compound of Preparation L (0.139 g; 0.72 mmol) and proceeding in analogy to Procedure D (96% yield) and Procedure B (53% yield), the title compound (0.135 g) was obtained after purification by CC (DCM-MeOH) as a yellowish solid.

¹H NMR (d6-DMSO) δ: 10.92 (s, 1H); 9.15 (s, 1H); 7.61 (s, 1H); 5.26 (t, J=6.1 Hz, 1H); 4.43 (s, 2H); 3.74-3.50 (m, 3H); 3.44 (m, 1H); 3.05 (s, 3H); 2.61-2.53 (overlapped m, 1H); 2.03 (m, 1H); 1.94 (m, 1H); 1.53 (s, 3H); 1.47-1.37 (m, 2H).

MS (ESI, m/z): 469.00 [M+H⁺] for C₂₀H₂₁N₂O₆FS₂; t_{R}=0.64 min.

### Example 5: (2R)—N-hydroxy-2-methyl-4-(2-((1-(methylamino)cyclopropyl)buta-1,3-diyn-1-yl)-6-oxo-4,6-dihydro-5H-thieno[2,3-c]pyrrol-5-yl)-2-(methylsulfonyl)butanamide

Starting from the compound of Preparation K (0.105 g; 0.24 mmol) and the compound of Preparation M (0.058 g; 0.27 mmol) and proceeding in analogy to Procedure D (70% yield) and Procedure B (42% yield), the title compound (0.010 g) was obtained after purification by CC (DCM-MeOH) as a yellowish solid.

¹H NMR (d6-DMSO) δ: 10.92 (s, 1H); 9.15 (s, 1H); 7.59 (s, 1H); 4.43 (s, 2H); 3.54 (m, 1H); 3.43 (m, 1H); 3.05 (s, 3H); 2.82 (br s, 1H); 2.61-2.47 (overlapped m, 1H); 2.33 (s, 3H); 1.94 (m, 1H); 1.53 (s, 3H); 1.01-0.98 (m, 2H); 0.91-0.88 (m, 2H).

MS (ESI, m/z): 450.03 [M+H⁺] for C₂₀H₂₃N₃O₅S₂; t_{R}=0.51 min.

### Example 6: (3R)-5-(5-(4-(hydroxyamino)-3-methyl-3-(methylsulfonyl)-4-oxobutyl)-6-oxo-5,6-dihydro-4H-thieno[2,3-c]pyrrol-2-yl)penta-2,4-diyn-1-yl 3-hydroxyazetidine-1-carboxylate

Starting from the compound of Preparation K (0.094 g; 0.22 mmol) and the compound of Preparation N (0.059 g; 0.26 mmol) and proceeding in analogy to Procedure D (46% yield) and Procedure B (38% yield), the title compound (0.019 g) was obtained after purification by CC (DCM-MeOH) as a yellowish solid.

¹H NMR (d6-DMSO) δ: 10.93 (s, 1H); 9.14 (s, 1H); 7.68 (s, 1H); 5.74 (d, J=6.5 Hz, 1H); 4.88 (s, 2H); 4.45 (s, 2H); 4.50-4.41 (overlapped m, 1H); 4.18-4.05 (m, 2H); 3.72-3.69 (m, 2H); 3.55 (m, 1H); 3.45 (m, 1H); 3.06 (s, 3H); 2.61-2.47 (overlapped m, 1H); 1.95 (m, 1H); 1.53 (s, 3H).

MS (ESI, m/z): 510.0 [M+H⁺] for C₂₁H₂₃N₃O₈S₂; t_{R}=0.62 min.

### Example 7: (2R)—N-hydroxy-2-methyl-4-(5-((1-(methylamino)cyclopropyl)buta-1,3-diyn-1-yl)-1-oxoisoindolin-2-yl)-2-(methylsulfonyl)butanamide

Starting from the compound of Preparation O (0.087 g; 0.2 mmol) and the compound of Preparation M (0.058 g; 0.27 mmol) and proceeding in analogy to Procedure D (72% yield) and Procedure F (40% yield), the title compound (0.025 g) was obtained after purification by prep-HPLC (Method 1), as a beige solid.

¹H NMR (d6-DMSO) δ: 11.29-10.11 (m, 1H); 9.31-8.99 (m, 1H); 7.79 (s, 1H); 7.66 (m, 1H); 7.63-7.61 (m, 1H); 4.50 (s, 2H); 3.61 (m, 1H); 3.48 (m, 1H); 3.07 (s, 3H); 2.84-2.80 (m, 1H); 2.59 (m, 1H); 2.35 (s, 3H); 1.97 (m, 1H); 1.55 (s, 3H); 1.00-0.98 (m, 2H); 0.89 (m, 2H).

MS (ESI, m/z): 444.16 [M+H⁺] for C₂₂H₂₅N₃O₅S; t_{R}=0.52 min.

### Example 8: (2R)—N-hydroxy-2-methyl-4-(5-((1-methylazetidin-3-yl)buta-1,3-diyn-1-yl)-1-oxoisoindolin-2-yl)-2-(methylsulfonyl)butanamide

### 8.i. (2R)-4-(5-(azetidin-3-ylbuta-1,3-diyn-1-yl)-1-oxoisoindolin-2-yl)-2-methyl-2-(methylsulfonyl)-N-((2RS)-(tetrahydro-2H-pyran-2-yl)oxy)butanamide

Starting from the compound of Preparation O (0.2 g; 0.45 mmol) and the compound of Preparation P (0.117 g; 0.6 mmol) and proceeding in analogy to Procedure D (36% yield), the title compound (0.084 g) was obtained, after purification by CC (DCM-MeOH), as a brown solid.

MS (ESI, m/z): 513.95 [M+H⁺] for C₂₆H₃₁N₃O₆S; t_{R}=0.59 min.

### 8.ii. (2R)-2-methyl-4-(5-((1-methylazetidin-3-yl)buta-1,3-diyn-1-yl)-1-oxoisoindolin-2-yl)-2-(methylsulfonyl)-N-((2RS)-(tetrahydro-2H-pyran-2-yl)oxy)butanamide

To a solution of intermediate 8.i (0.084 g; 0.3 mmol) in DCM (3 mL) were added formaldehyde (37% aq., 0.04 mL, 0.53 mmol) and NaBH(OAc)₃ (0.22 g, 0.98 mmol). The reaction mixture was stirred at rt for 1 h. Sat. aq. NaHCO₃ (5 mL) was added. The two layers were separated and the aq. layer was extracted with DCM-MeOH (9-1, 3×5 mL). The evaporation residue was purified by CC (Hept-EA-MeOH) to afford the title product (0.034 g; 39% yield) as a brown solid.

MS (ESI, m/z): 569.17 [M+MeCN+H⁺] for C₂₇H₃₃N₃O₆S; t_{R}=0.59 min.

### 8.iii. (2R)—N-hydroxy-2-methyl-4-(5-((1-methylazetidin-3-yl)buta-1,3-diyn-1-yl)-1-oxoisoindolin-2-yl)-2-(methylsulfonyl)butanamide

Starting from intermediate 8.ii (0.034 g; 0.18 mmol) and proceeding in analogy to Procedure F (31% yield), the title compound (0.009 g) was obtained, after purification by prep-HPLC (Method 1), as a beige solid.

¹H NMR (d6-DMSO) δ: 10.98-10.92 (m, 1H); 9.19-9.13 (m, 1H); 7.81 (s, 1H); 7.67 (m, 1H); 7.64 (m, 1H); 4.50 (s, 2H); 3.64-3.58 (m, 1H); 3.55-3.45 (m, 3H); 3.39 (m, 1H); 3.07 (s, 3H); 3.03 (t, J=6.6 Hz, 2H); 2.61-2.58 (m, 1H); 2.20 (s, 3H); 2.03-1.94 (m, 1H); 1.55 (s, 3H).

MS (ESI, m/z): 485.0 [M+MeCN+H+] for C₂₂H₂₅N₃O₅S; t_{R}=0.50 min.

### Example 9: (3R)-5-(2-(4-(hydroxyamino)-3-methyl-3-(methylsulfonyl)-4-oxobutyl)-1-oxoisoindolin-5-yl)penta-2,4-diyn-1-yl 3-hydroxyazetidine-1-carboxylate

Starting from the compound of Preparation O (0.08 g; 0.18 mmol) and the compound of Preparation N (0.059 g; 0.26 mmol) and proceeding in analogy to Procedure D (65% yield) and Procedure F (62% yield), the title compound (0.036 g) was obtained after purification by prep-HPLC (Method 1), as a beige solid.

¹H NMR (d6-DMSO) δ: 10.92-10.58 (m, 1H); 9.15 (m, 1H); 7.85 (s, 1H); 7.68 (s, 2H); 5.75 (d, J=6.5 Hz, 1H); 4.87 (s, 2H); 4.51 (s, 2H); 4.46 (m, 1H); 4.21-4.06 (m, 2H); 3.71-3.70 (m, 2H); 3.61 (m, 1H); 3.52-3.47 (m, 1H); 3.07 (s, 3H); 2.65-2.58 (m, 1H); 2.02-1.95 (m, 1H); 1.55 (s, 3H).

MS (ESI, m/z): 504.0 [M+H⁺] for C₂₃H₂₅N₃O₈S; t_{R}=0.63 min.

### Example 10: (2R)-4-(5-(2-fluoro-4-methoxyphenyl)-1-oxoisoindolin-2-yl)-N-hydroxy-2-methyl-2-(methylsulfonyl)butanamide

Starting from the compound of Preparation Q (0.13 g; 0.266 mmol) and 2-fluoro-4-methoxyphenylboronic acid (0.050 g; 0.29 mmol) and proceeding in analogy to Procedure E (71% yield) and Procedure B (15% yield), the title compound (0.012 g) was obtained after trituration in Et₂O, as a brownish oil.

MS (ESI, m/z): 451.1 [M+H⁺] for C₂₁H₂₃N₂O₆FS; t_{R}=0.72 min.

### Example 11: (2R)—N-hydroxy-2-methyl-4-(2-((1-methylazetidin-3-yl)buta-1,3-diyn-1-yl)-6-oxo-4,6-dihydro-5H-thieno[2,3-c]pyrrol-5-yl)-2-(methylsulfonyl)butanamide

### 11.i. (2R)-4-(2-(azetidin-3-ylbuta-1,3-diyn-1-yl)-6-oxo-4,6-dihydro-5H-thieno[2,3-c]pyrrol-5-yl)-2-methyl-2-(methylsulfonyl)-N-((2RS)-(tetrahydro-2H-pyran-2-yl)oxy)butanamide

Starting from the compound of Preparation K (0.334 g; 0.759 mmol) and the compound of Preparation P (0.171 g; 0.87 mmol) and proceeding in analogy to Procedure D (28% yield), the title compound (0.111 g) was obtained, after purification by CC (DCM-MeOH-aq. NH₄OH), as a brownish solid.

MS (ESI, m/z): 520.07 [M+H⁺] for C₂₄H₂₉N₃O₆S₂; t_{R}=0.58 min.

### 11.ii. (2R)—N-hydroxy-2-methyl-4-(2-((1-methylazetidin-3-yl)buta-1,3-diyn-1-yl)-6-oxo-4,6-dihydro-5H-thieno[2,3-c]pyrrol-5-yl)-2-(methylsulfonyl)butanamide

Starting from intermediate 11.i (0.056 g; 0.11 mmol) and proceeding successively in analogy to Example 8, step 8.ii (49% yield) and Procedure F (28% yield), the title compound (0.006 g) was obtained, after purification by prep-HPLC (Method 1), as a yellowish solid.

¹H NMR (d6-DMSO) δ: 10.92 (br. s, 1H); 9.15 (br. s, 1H); 7.61 (s, 1H); 4.44 (s, 2H); 3.58-3.38 (m, 5H); 3.06 (s, 3H); 3.03-2.99 (m, 2H); 2.61-2.47 (overlapped m, 1H); 2.18 (s, 3H); 1.94 (m, 1H); 1.52 (s, 3H).

MS (ESI, m/z): 491.0 [M+MeCN+H⁺] for C₂₀H₂₃N₃O₅S₂; t_{R}=0.49 min.

### Example 12: (2R)—N-hydroxy-4-(5-((1-(hydroxymethyl)cyclopropyl)buta-1,3-diyn-1-yl)-1-oxoisoindolin-2-yl)-2-methyl-2-(methylsulfonyl)butanamide

Starting from the compound of Preparation O (0.08 g; 0.18 mmol) and the compound of Preparation R (0.045 g; 0.26 mmol) and proceeding in analogy to Procedure D (>95% yield) and Procedure F (67% yield), the title compound (0.043 g) was obtained after purification by prep-HPLC (Method 1), as a beige solid.

¹H NMR (d6-DMSO) δ: 10.95 (s, 1H); 9.15 (s, 1H); 7.78 (s, 1H); 7.65 (m, 1H); 7.62-7.60 (m, 1H); 5.06 (t, J=6.1 Hz, 1H); 4.50 (s, 2H); 3.60 (m, 1H); 3.48 (m, 1H); 3.41 (d, J=6.0 Hz, 2H); 3.07 (s, 3H); 2.62-2.58 (m, 1H); 1.97 (m, 1H); 1.55 (s, 3H); 0.98-0.96 (m, 2H); 0.92-0.90 (m, 2H).

MS (ESI, m/z): 445.0.0 [M+H⁺] for C₂₂H₂₄N₂O₆S; t_{R}=0.66 min.

### Example 13: (2R)-4-(5-((1-aminocyclopropyl)buta-1,3-diyn-1-yl)-1-oxoisoindolin-2-yl)-N-hydroxy-2-methyl-2-(methylsulfonyl)butanamide

Starting from the compound of Preparation O (0.1 g; 0.23 mmol) and the compound of Preparation J (0.055 g; 0.28 mmol) and proceeding in analogy to Procedure D (81% yield) and Procedure F (57% yield), the title compound (0.043 g) was obtained after purification by prep-HPLC (Method 1), as a beige solid.

¹H NMR (d6-DMSO) δ: 11.05-10.83 (m, 1H); 9.15 (m, 1H); 7.78 (s, 1H); 7.66 (m, 1H); 7.61 (m, 1H); 4.50 (s, 2H); 3.60 (m, 1H); 3.51-3.43 (m, 1H); 3.07 (s, 3H); 2.60 (m, 1H); 2.00-1.92 (m, 1H); 1.55 (s, 3H); 0.99 (m, 2H); 0.88 (m, 2H).

MS (ESI, m/z): 471.04 [M+H⁺] for C₂₁H₂₃N₃O₅S; t_{R}=0.50 min.

### Example 14: (2R)-4-(2-((4-((3-fluoroazetidin-1-yl)methyl)phenyl)ethynyl)-6-oxo-4,6-dihydro-5H-thieno[2,3-c]pyrrol-5-yl)-N-hydroxy-2-methyl-2-(methylsulfonyl)butanamide

Starting from the compound of Preparation K (0.15 g; 0.34 mmol) and the compound of Preparation S (0.17 g; 0.58 mmol) and proceeding in analogy to Procedure G (43% yield) and Procedure B (16% yield), the title compound (0.012 g) was obtained after purification by CC (DCM-MeOH), as a beige solid.

¹H NMR (d6-DMSO) δ: 10.98-10.90 (br. s, 1H); 9.17 (s, 1H); 7.56-7.53 (m, 3H); 7.36 (d, J=8.2 Hz, 2H); 5.19 (m, 1H); 4.51-4.43 (m, 2H); 3.67 (s, 2H); 3.61-3.51 (m, 3H); 3.45 (m, 1H); 3.16 (m, 2H); 3.08 (s, 3H); 2.60 (m, 1H); 1.97 (m, 1H); 1.55 (s, 3H).

MS (ESI, m/z): 519.98 [M+H⁺] for C₂₄H₂₆N₃O₅FS₂; t_{R}=0.55 min.

### Example 15: (2R)—N-hydroxy-2-methyl-2-(methylsulfonyl)-4-(2-((1-(oxetan-3-yl)azetidin-3-yl)buta-1,3-diyn-1-yl)-6-oxo-4,6-dihydro-5H-thieno[2,3-c]pyrrol-5-yl)butanamide

Starting from intermediate 11.i (0.055 g; 0.11 mmol) and oxetanone (0.025 g; 0.33 mmol) and proceeding in analogy to Example 8, step 8.ii (45% yield) and Procedure B (15% yield), the title compound (0.003 g) was obtained after purification by prep-HPLC (Method 1), as a beige solid.

¹H NMR (d6-DMSO) δ: 7.59 (s, 1H); 4.53 (t, J=6.6 Hz, 2H); 4.43 (s, 2H); 4.33-4.29 (m, 2H); 3.67 (m, 1H); 3.54 (m, 1H); 3.30 (m, 1H); 3.18-3.12 (m, 2H); 3.04 (s, 3H); 3.04-2.99 (overlapped m, 1H); 2.61-2.54 (overlapped m, 1H); 1.94 (m, 1H); 1.52 (s, 3H).

MS (ESI, m/z): 492.0 [M+H⁺] for C₂₂H₂₅N₃O₆S₂; t_{R}=0.49 min.

### Example 16: ((1R,2R)-1-fluoro-2-((5-((3R)-4-(hydroxyamino)-3-methyl-3-(methylsulfonyl)-4-oxobutyl)-6-oxo-5,6-dihydro-4H-thieno[2,3-c]pyrrol-2-yl)buta-1,3-diyn-1-yl)cyclopropyl)methyl dihydrogen phosphate

Starting from the compound of Preparation K (0.103 g; 0.23 mmol) and the compound of Preparation T (0.113 g; 0.29 mmol) and proceeding successively in analogy to Procedure D (69% yield) and Procedure F (25% yield), the title compound (0.021 g) was obtained, after purification by prep-HPLC (Method 2), as a yellowish solid.

¹H NMR (d6-DMSO) δ: 10.92 (s, 1H); 9.15 (s, 1H); 7.62 (s, 1H); 4.43 (s, 2H); 4.17-4.02 (m, 2H); 3.54 (m, 1H); 3.43 (m, 1H); 3.05 (s, 3H); 2.61-2.47 (overlapped m, 1H); 2.20 (m, 1H); 1.94 (m, 1H); 1.57-1.51 (overlapped m, 2H); 1.53 (s, 3H).

MS (ESI, m/z): 548.90 [M+H⁺] for C₂₀H₂₂N₂O₉FPS₂; t_{R}=0.53 min.

### Example 17: (2R)—N-hydroxy-4-(2-((1-(2-hydroxyethyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide

Starting from the compound of Preparation K (0.09 g; 0.20 mmol) and the compound of Preparation U (0.075 g; 0.24 mmol) and proceeding successively in analogy to Procedure D (87% yield), Reference Example 2, step RE2.ii (41% yield) and Procedure F (29% yield), the title compound (0.010 g) was obtained, after purification by prep-HPLC (Method 1), as a beige solid.

¹H NMR (d6-DMSO) δ: 10.9 (br. s, 1H); 9.15 (br. s, 1H); 7.61 (s, 1H); 4.44 (s, 2H); 4.42 (t, J=5.5 Hz, 1H); 3.58-3.50 (m, 3H); 3.47-3.40 (m, 2H); 3.35-3.31 (overlapped m, 2H); 3.09-3.04 (overlapped m, 2H); 3.06 (s, 3H); 2.60-2.46 (overlapped m, 1H); 2.43 (t, J=6.0 Hz, 2H); 1.94 (m, 1H); 1.52 (s, 3H).

MS (ESI, m/z): 480.0 [M+H⁺] for C₂₁H₂₅N₃O₆S₂; t_{R}=0.48 min.

### Example 18: (2R)-4-(5-(((1R,2R)-2-fluoro-2-(hydroxymethyl)cyclopropyl)buta-1,3-diyn-1-yl)-1-oxoisoindolin-2-yl)-N-hydroxy-2-methyl-2-(methylsulfonyl)butanamide

Starting from the compound of Preparation O (0.1 g; 0.23 mmol) and the compound of Preparation L (0.063 g; 0.33 mmol) and proceeding in analogy to Procedure D (81% yield) and Procedure F (14% yield), the title compound (0.012 g) was obtained after purification by prep-HPLC (Method 1), as a beige solid.

¹H NMR (d6-DMSO) δ: 10.82 (br. s, 1H); 9.13 (br. s, 1H); 7.81 (s, 1H); 7.65 (m, 2H); 5.26 (t, J=6.1 Hz, 1H); 4.50 (s, 2H); 3.75-3.56 (m, 3H); 3.51-3.42 (m, 1H); 3.06 (s, 3H); 2.64-2.54 (overlapped m, 1H); 2.03-1.92 (m, 2H); 1.55 (s, 3H); 1.47-1.34 (m, 2H).

MS (ESI, m/z): 463.01 [M+H⁺] for C₂₂H₂₃N₂O₆FS; t_{R}=0.65 min.

### Example 19: (2R)—N-hydroxy-4-(2-(((1S,2S)-2-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)butanamide

### 19.i. (2R)-4-(2-(((1S,2S)-2-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)-N-(((2RS)-tetrahydro-2H-pyran-2-yl)oxy)butanamide

Starting from the compound of Preparation K (0.3 g; 0.68 mmol) and the (1S,2S)-configurated compound of Preparation H (0.156 g; 0.72 mmol) and proceeding in analogy to Procedure D (79% yield), the title compound (0.286 g) was obtained, after purification by CC (DCM-MeOH), as a yellowish foam.

¹H NMR (d6-DMSO) δ: (mixture of diastereomers) 11.34 (s, 0.5H); 11.32 (s, 0.5H); 7.57 (s, 0.5H); 7.56 (s, 0.5H); 4.85 (m, 0.5H); 4.72 (t, J=5.7 Hz, 1H); 4.61 (m, 0.5H); 4.47-4.37 (m, 2H); 3.99 (m, 1H); 3.60-3.51 (m, 2H); 3.46-3.41 (m, 2H); 3.26 (m, 1H); 3.05 (s, 1.5H); 3.03 (s, 1.5H); 2.65-2.47 (overlapped m, 1H); 1.96 (m, 1H); 1.67-1.61 (m, 2H); 1.55 (s, 1.5H); 1.54 (s, 1.5H); 1.55-1.45 (overlapped m, 6H); 0.97 (m, 1H); 0.92 (m, 1H).

MS (ESI, m/z): 535.0 [M+H⁺] for C₂₅H₃₀N₂O₇S₂; t_{R}=0.77 min.

### 19.ii. (2R)—N-hydroxy-4-(2-(((1S,2S)-2-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)butanamide

Starting from intermediate 19.i (0.093 g; 0.17 mmol) and proceeding in analogy to Procedure B (56% yield), the title compound (0.043 g) was obtained, after purification by CC (DCM-MeOH), as a yellowish solid.

¹H NMR (d6-DMSO) δ: 10.92 (s, 1H); 9.14 (s, 1H); 7.56 (s, 1H); 4.72 (t, J=5.7 Hz, 1H); 4.43 (s, 2H); 3.54 (m, 1H); 3.39-3.46 (m, 2H); 3.26 (m, 1H); 3.05 (s, 3H); 2.60-2.47 (overlapped m, 1H); 1.94 (m, 1H); 1.56-1.43 (overlapped m, 2H); 1.52 (s, 3H); 0.97 (m, 1H); 0.92 (m, 1H).

MS (ESI, m/z): 451.00 [M+H⁺] for C₂₀H₂₂N₂O₆S₂; t_{R}=0.66 min.

### Example 20: ((1S,2S)-2-((5-((3R)-4-(hydroxyamino)-3-methyl-3-(methylsulfonyl)-4-oxobutyl)-6-oxo-5,6-dihydro-4H-thieno[2,3-c]pyrrol-2-yl)buta-1,3-diyn-1-yl)cyclopropyl)methyl dimethylglycinate

### 20.i. ((1S,2S)-2-((5-((3R)-3-methyl-3-(methylsulfonyl)-4-oxo-4-(((tetrahydro-2H-pyran-2-yl)oxy)amino)butyl)-6-oxo-5,6-dihydro-4H-thieno[2,3-c]pyrrol-2-yl)buta-1,3-diyn-1-yl)cyclopropyl)methyl dimethylglycinate

To a solution of intermediate 19.i (0.180 g; 0.34 mmol) in DMF (2.6 mL) were added N,N-dimethylglycine (0.036 g; 0.34 mmol), EDC (0.072 g; 0.38 mmol), HOBT (0.060 g; 0.39 mmol) and TEA (0.08 mL, 0.58 mmol). The reaction mixture was stirred 2 days. The reaction mixture was diluted with DCM (30 mL), washed with 15% aq. NaHSO₄ (10 mL) and sat. aq. NaHCO₃ (10 mL). The evaporation residue was purified by CC (DCM-MeOH) to afford the title product (0.158 g, 76% yield) as a yellow gum.

¹H NMR (d6-DMSO) δ (mixture of diastereomers): 11.34 (s, 0.5H); 11.32 (s, 0.5H); 7.58 (s, 0.5H); 7.58 (s, 0.5H); 4.85 (m, 0.5H); 4.61 (m, 0.5H); 4.48-4.36 (m, 2H); 4.05-3.94 (m, 2H); 3.87 (m, 1H); 3.55 (m, 2H); 3.43 (m, 2H); 3.19 (s, 2H); 3.05 (s, 1.5H); 3.03 (s, 1.5H); 2.58-2.43 (overlapped m, 1H); 2.25 (s, 6H); 1.97 (m, 1H); 1.71 (m, 1H); 1.66-1.60 (m, 2H); 1.59-1.45 (overlapped m, 4H); 1.55 (s, 1.5H); 1.54 (s, 1.5H); 1.10 (m, 1H); 1.02 (m, 1H).

MS (ESI, m/z): 620.09 [M+H⁺] for C₂₉H₃₇N₃O₈S₂; t_{R}=0.67 min.

### 20.ii. ((1S,2S)-2-((5-((3R)-4-(hydroxyamino)-3-methyl-3-(methylsulfonyl)-4-oxobutyl)-6-oxo-5,6-dihydro-4H-thieno[2,3-c]pyrrol-2-yl)buta-1,3-diyn-1-yl)cyclopropyl)methyl dimethylglycinate

Starting from intermediate 20.i (0.158 g; 0.26 mmol) and proceeding in analogy to Procedure H (34% yield), the title compound (0.049 g) was obtained, after purification by prep-HPLC (Method 3), as a yellowish solid.

¹H NMR (d6-DMSO) δ: 10.92 (s, 1H); 9.15 (s, 1H); 7.58 (s, 1H); 4.43 (s, 2H); 4.03 (m, 1H); 3.87 (dd, J=7.7, 11.7 Hz, 1H); 3.53 (dd, J=4.6, 9.3 Hz, 1H); 3.43 (m, 1H); 3.19 (s, 2H); 3.05 (s, 3H); 2.58-2.44 (overlapped m, 1H); 2.25 (s, 6H); 1.94 (n, 1H); 1.71 (m, 1H); 1.63 (m, 1H); 1.52 (s, 3H); 1.10 (m, 1H); 1.02 (m, 1H).

MS (ESI, m/z): 536.05 [M+H⁺] for C₂₅H₂₉N₃O₇S₂; t_{R}=0.59 min.

### Example 21: (2R)-4-(2-(((1S,3R,4S)-3,4-dihydroxycyclopentyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide

Starting from the compound of Preparation K (0.15 g; 0.34 mmol) and the compound of Preparation V (0.073 g; 0.38 mmol) and proceeding successively in analogy to Procedure D (54% yield) and Procedure F (38% yield), the title compound (0.033 g) was obtained, after purification by CC (DCM-MeOH), as a yellowish solid.

¹H NMR (d6-DMSO) δ: 10.92 (br. s, 1H); 9.13 (br. s, 1H); 7.57 (s, 1H); 4.57 (d, J=4.3 Hz, 2H); 4.43 (s, 2H); 3.97-3.92 (m, 2H); 3.54 (m, 1H); 3.43 (m, 1H); 3.20 (m, 1H); 3.05 (s, 3H); 2.60-2.45 (overlapped m, 1H); 1.98-1.90 (m, 3H); 1.82-1.75 (m, 2H); 1.52 (s, 3H).

MS (ESI, m/z): 481.00 [M+H⁺] for C₂₁H₂₄N₂O₇S₂; t_{R}=0.60 min.

### Example 22: (2R)-4-(2-(((1S,2S)-2-((2R)-1,2-dihydroxyethyl)cyclopropyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide

Starting from the compound of Preparation K (0.122 g; 0.27 mmol) and the compound of Preparation W (0.058 g; 0.28 mmol) and proceeding successively in analogy to Procedure D (76% yield) and Procedure B (37% yield), the title compound was obtained, after purification by CC (DCM-MeOH), as a yellowish solid (0.037 g).

¹H NMR (d6-DMSO) δ: 10.91 (br. s, 1H); 9.14 (s, 1H); 7.56 (s, 1H); 4.72 (d, J=4.9 Hz, 1H); 4.62 (t, J=5.3 Hz, 1H); 4.42 (s, 2H); 3.54 (m, 1H); 3.43 (m, 1H); 3.38-3.28 (overlapped m, 3H); 3.05 (s, 3H); 2.59-2.44 (overlapped m, 1H); 1.94 (m, 1H); 1.57-1.51 (overlapped m, 1H); 1.52 (s, 3H); 1.42 (m, 1H); 0.99 (m, 1H); 0.88 (m, 1H).

MS (ESI, m/z): 481.00 [M+H⁺] for C₂₁H₂₄N₂O₇S₂; t_{R}=0.59 min.

### Example 23: (2R)-4-(2-((1-(2-fluoroethyl)azetidin-3-yl)buta-1,3-diyn-1-yl)-6-oxo-4,6-dihydro-5H-thieno[2,3-c]pyrrol-5-yl)-N-hydroxy-2-methyl-2-(methylsulfonyl)butanamide

Starting from the compound of Preparation K (0.11 g; 0.25 mmol) and the compound of Preparation X (0.063 g; 0.32 mmol) and proceeding successively in analogy to Procedure D (77% yield) and Procedure F (59% yield), the title compound was obtained, after purification by prep-HPLC (Method 1), as a beige solid (0.055 g).

¹H NMR (d6-DMSO) δ: 10.50 (m, 1H); 9.15 (m, 1H); 7.62 (s, 1H); 4.42-4.46 (overlapped m, 1H); 4.45 (s, 2H); 4.34 (m, 1H); 3.41-3.60 (m, 5H); 3.15-3.11 (m, 2H); 3.06 (s, 3H); 2.70 (m, 1H); 2.64 (m, 1H); 2.57 (m, 1H); 1.95 (m, 1H); 1.53 (s, 3H).

MS (ESI, m/z): 481.9 [M+H⁺] for C₂₁H₂₄N₃O₅FS₂; t_{R}=0.51 min.

### Example 24: (R)—N-hydroxy-4-(2-((1-isopropylazetidin-3-yl)buta-1,3-diyn-1-yl)-6-oxo-4,6-dihydro-5H-thieno[2,3-c]pyrrol-5-yl)-2-methyl-2-(methylsulfonyl)butanamide

Starting from the compound of Preparation K (0.11 g; 0.27 mmol) and the compound of Preparation Y (0.077 g; 0.37 mmol) and proceeding successively in analogy to Procedure D (96% yield) and Procedure F (59% yield), the title compound was obtained, after purification by prep-HPLC (Method 1), as a beige solid (0.065 g).

¹H NMR (d6-DMSO) δ: 10.28 (m, 1H); 9.20 (m, 1H); 7.62 (s, 1H); 4.45 (s, 2H); 3.55 (m, 1H); 3.40-3.48 (m, 3H); 3.31-3.39 (overlapped m, 1H); 3.06 (s, 3H); 2.97-3.01 (m, 2H); 2.58 (m, 1H); 2.25 (m, 1H); 1.95 (m, 1H); 1.53 (s, 3H); 0.83 (d, J=6.2 Hz, 6H).

MS (ESI, m/z): 478.0 [M+H⁺] for C₂₂H₂₇N₃O₅S₂; t_{R}=0.53 min.

### Example 25: (2R)—N-hydroxy-4-(2-((1-(2-hydroxy-2-methylpropyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide

Starting from the compound of Preparation K (0.15 g; 0.34 mmol) and the compound of Preparation Z (0.090 g; 0.39 mmol) and proceeding successively in analogy to Procedure D (93% yield) and Procedure F (59% yield), the title compound was obtained, after purification by prep-HPLC (Method 1), as a beige solid (0.107 g).

¹H NMR (d6-DMSO) δ: 10.91 (m, 1H); 9.14 (m, 1H); 7.61 (s, 1H); 4.44 (s, 2H); 4.07 (s, 1H); 3.60-3.52 (m, 3H); 3.49-3.40 (m, 2H); 3.12-3.08 (m, 2H); 3.06 (s, 3H); 2.57 (ddd, J=6.6, 9.6, 13.2 Hz, 1H); 2.28 (s, 2H); 1.95 (m, 1H); 1.52 (s, 3H); 1.02 (s, 6H).

MS (ESI, m/z): 508.1 [M+H⁺] for C₂₃H₂₉N₃O₆S₂; t_{R}=0.51 min.

### Example 26: (2R)—N-hydroxy-2-methyl-2-(methylsulfonyl)-4-(2-((4-(1-(oxetan-3-yl)azetidin-3-yl)phenyl)ethynyl)-6-oxo-4,6-dihydro-H-thieno[2,3-c]pyrrol-5-yl)butanamide

Starting from the compound of Preparation K (0.09 g; 0.2 mmol) and the compound of Preparation AA (0.090 g; 0.29 mmol) and proceeding successively in analogy to Procedure A (87% yield) and Procedure F (27% yield), the title compound was obtained, after purification by prep-HPLC (Method 1), as an off-white solid (0.025 g).

¹H NMR (d6-DMSO) δ: 10.90 (m, 1H); 9.17 (m, 1H); 7.57 (d, J=8.1 Hz, 2H); 7.54 (s, 1H); 7.47 (d, J=8.3 Hz, 2H); 4.56-4.60 (m, 2H); 4.47 (d, J=1.1 Hz, 2H); 4.38-4.41 (m, 2H); 3.76 (m, 1H); 3.64-3.72 (m, 3H); 3.57 (m, 1H); 3.44 (m, 1H); 3.19-3.21 (m, 2H); 3.08 (s, 3H); 2.59 (m, 1H); 1.97 (m, 1H); 1.55 (s, 3H).

MS (ESI, m/z): 544.2 [M+H⁺] for C₂₆H₂₉N₃O₆S₂; t_{R}=0.55 min.

### Example 27: (2R)—N-hydroxy-2-methyl-2-(methylsulfonyl)-4-(6-oxo-2-((1-(tetrahydro-2H-pyran-4-yl)azetidin-3-yl)buta-1,3-diyn-1-yl)-4,6-dihydro-5H-thieno[2,3-c]pyrrol-5-yl)butanamide

Starting from the compound of Preparation K (0.15 g; 0.34 mmol) and the compound of Preparation AB (0.096 g; 0.39 mmol) and proceeding successively in analogy to Procedure D (59% yield) and Procedure F (27% yield), the title compound was obtained, after purification by prep-HPLC (Method 1), as an off-white solid (0.025 g).

¹H NMR (d6-DMSO) δ: 10.79 (br.s, 1H); 9.08 (br. s, 1H); 7.61 (s, 1H); 4.44 (s, 2H); 3.78 (dt, J=3.9, 11.5 Hz, 2H); 3.55 (m, 1H); 3.50-3.46 (m, 2H); 3.45-3.40 (m, 2H); 3.29-3.23 (m, 2H); 3.05 (s, 3H); 3.04-3.00 (m, 2H); 2.56 (m, 1H); 2.21 (m, 1H); 1.94 (m, 1H); 1.58-1.53 (m, 2H); 1.52 (s, 3H); 1.13-1.07 (m, 2H).

MS (ESI, m/z): 520.2 [M+H⁺] for C₂₄H₂₉N₃O₆S₂; t_{R}=0.51 min.

### Example 28: (2R)—N-hydroxy-2-methyl-2-(methylsulfonyl)-4-(2-((1-(oxetan-3-ylmethyl)azetidin-3-yl)buta-1,3-diyn-1-yl)-6-oxo-4,6-dihydro-5H-thieno[2,3-c]pyrrol-5-yl)butanamide

Starting from the compound of Preparation K (0.15 g; 0.34 mmol) and the compound of Preparation AC (0.095 g; 0.41 mmol) and proceeding successively in analogy to Procedure D (73% yield) and Procedure B (41% yield), the title compound was obtained, after purification by CC (DCM-MeOH), as a yellowish foam (0.025 g).

¹H NMR (d6-DMSO) δ: 10.94 (br. s, 1H); 9.16 (br. s, 1H); 7.62 (s, 1H); 4.58 (dd, J=5.9, 7.8 Hz, 2H); 4.44 (s, 2H); 4.23 (t, J=6.0 Hz, 2H); 3.58-3.41 (m, 5H); 3.06 (s, 3H); 3.06-3.03 (overlapped m, 2H); 2.91 (m, 1H); 2.69-2.64 (m, 2H); 2.58 (m, 1H); 1.95 (m, 1H); 1.53 (s, 3H).

MS (ESI, m/z): 506.05 [M+H⁺] for C₂₃H₂₇N₃O₆S₂; t_{R}=0.50 min.

### Example 29: (2R)—N-hydroxy-2-methyl-2-(methylsulfonyl)-4-(2-((4-(1-(oxetan-3-yl)azetidin-3-yl)phenyl)ethynyl)-6-oxo-4,6-dihydro-H-thieno[2,3-c]pyrrol-5-yl)butanamide

Starting from the compound of Preparation K (0.153 g; 0.35 mmol) and the compound of Preparation AD (0.128 g; 0.39 mmol) and proceeding successively in analogy to Procedure A (75% yield) and Procedure B (27% yield), the title compound was obtained, after purification by CC (DCM-MeOH), as a yellow solid (0.040 g).

¹H NMR (d6-DMSO) δ: 10.94 (br. s, 1H); 9.18 (br. s, 1H); 7.55-7.53 (m, 3H); 7.44-7.38 (m, 2H); 4.60 (dd, J=5.8, 7.8 Hz, 2H); 4.46 (s, 2H); 4.27 (t, J=6.0 Hz, 2H); 3.66-3.53 (m, 4H); 3.44 (m, 1H); 3.11-3.07 (overlapped m, 2H); 3.07 (s, 3H); 2.96 (m, 1H); 2.74-2.70 (m, 2H); 2.59 (m, 1H); 1.96 (m, 1H); 1.54 (s, 3H).

MS (ESI, m/z): 558.13 [M+H⁺] for C₂₇H₃₁N₃O₆S₂; t_{R}=0.56 min.

### Example 30: (2R)-4-(2-(((1S,2S)-2-((3-fluoroazetidin-1-yl)methyl)cyclopropyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide

Starting from the compound of Preparation K (0.148 g; 0.34 mmol) and the compound of Preparation AE (0.094 g; 0.40 mmol) and proceeding successively in analogy to Procedure D (52% yield) and Procedure B (32% yield), the title compound was obtained, after purification prep-HPLC (Method 1), as an off-white solid (0.028 g).

¹H NMR (d6-DMSO) δ: 10.88 (br. s, 1H); 9.03 (br. s, 1H); 7.56 (s, 1H); 5.21-5.06 (m, 1H); 4.46-4.39 (m, 2H); 3.59-3.51 (m, 3H); 3.42 (m, 1H); 3.14-3.05 (overlapped m, 2H); 3.05 (s, 3H); 2.59-2.44 (overlapped m, 2H); 2.29 (dd, J=7.2, 12.3 Hz, 1H); 1.92 (m, 1H); 1.52-1.49 (overlapped m, 1H); 1.50 (s, 3H); 1.27 (m, 1H); 0.98 (m, 1H); 0.85 (m, 1H).

MS (ESI, m/z): 508.11 [M+H⁺] for C₂₃H₂₆N₃O₅FS₂; t_{R}=0.55 min.

### Example 31: (2R)—N-hydroxy-4-(2-((1-((1s,3s)-3-hydroxycyclobutyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide

Starting from the compound of Preparation K (0.15 g; 0.34 mmol) and the (1s,3s)-isomer compound of Preparation AF (0.107 g; 0.4 mmol) and proceeding successively in analogy to Procedure D (46% yield) and Procedure F (55% yield), the title compound was obtained, after purification by prep-HPLC (Method 1), as a yellowish foam (0.041 g).

¹H NMR (d6-DMSO) δ: 10.93 (br. s, 1H); 9.15 (br. s, 1H); 7.61 (s, 1H); 4.95 (d, J=6.8 Hz, 1H); 4.44 (s, 2H); 3.73 (m, 1H); 3.55 (m, 1H); 3.46-3.36 (m, 4H); 3.08-2.99 (overlapped m, 2H); 3.06 (s, 3H); 2.61-2.44 (overlapped m, 2H); 2.22-2.15 (m, 2H); 1.95 (m, 1H); 1.60-1.53 (overlapped m, 2H); 1.53 (s, 3H).

MS (ESI, m/z): 506.05 [M+H⁺] for C₂₃H₂₇N₃O₆S₂; t_{R}=0.49 min.

### Example 32: (2R)—N-hydroxy-4-(2-((1-((1r,3r)-3-hydroxycyclobutyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide

Starting from the compound of Preparation K (0.15 g; 0.34 mmol) and the (1r,3r)-isomer compound of Preparation AF (0.107 g; 0.4 mmol) and proceeding successively in analogy to Procedure D (75% yield) and Procedure F (56% yield), the title compound was obtained, after purification by prep-HPLC (Method 1), as a yellowish foam (0.071 g).

¹H NMR (d6-DMSO) δ: 10.91 (br. s, 1H); 9.56 (br. s, 1H); 7.61 (s, 1H); 4.95 (d, J=6.4 Hz, 1H); 4.44 (s, 2H); 4.16 (hex, J=6.7 Hz, 1H); 3.54 (m, 1H); 3.47-3.37 (m, 4H); 3.05 (s, 3H); 2.95-2.90 (m, 2H); 2.87 (m, 1H); 2.58 (m, 1H); 1.97-1.90 (m, 3H); 1.78-1.73 (m, 2H); 1.52 (s, 3H).

MS (ESI, m/z): 506.1 [M+H⁺] for C₂₃H₂₇N₃O₆S₂; t_{R}=0.49 min.

### Example 33: (2R)—N-hydroxy-4-(5-((4-(2-hydroxyethyl)phenyl)ethynyl)-1-oxoisoindolin-2-yl)-2-methyl-2-(methylsulfonyl)butanamide

Starting from the compound of Preparation O (0.05 g; 0.115 mmol) and 2-(4-iodophenyl)ethan-1-ol (0.037 g; 0.138 mmol) and proceeding successively in analogy to Procedure G (65% yield) and Procedure F (47% yield), the title compound was obtained, after purification by prep-HPLC (Method 2), as a white solid (0.012 g).

¹H NMR (d6-DMSO) δ: 10.96 (s, 1H); 9.17 (s, 1H); 7.79 (s, 1H); 7.69 (m, 1H); 7.63 (m, 1H); 7.51 (d, J=8.1 Hz, 2H); 7.31 (d, J=8.1 Hz, 2H); 4.69 (t, J=5.2 Hz, 1H); 4.52 (d, J=1.9 Hz, 2H); 3.63 (m, 3H); 3.45-3.51 (m, 1H); 3.08 (s, 3H); 2.77 (t, J=6.8 Hz, 2H); 2.62-2.58 (m, 1H); 2.01-1.96 (m, 1H); 1.56 (s, 3H).

MS (ESI, m/z): 471.1 [M+H⁺] for C₂₄H₂₆N₂O₆S; t_{R}=0.67 min.

### Example 34: 3-hydroxypropyl (2R)-3-((5-(4-(hydroxyamino)-3-methyl-3-(methylsulfonyl)-4-oxobutyl)-6-oxo-5,6-dihydro-4H-thieno[2,3-c]pyrrol-2-yl)buta-1,3-diyn-1-yl)azetidine-1-carboxylate

Starting from the compound of Preparation K (0.132 g; 0.3 mmol) and the compound of Preparation AG, (0.138 g; 0.36 mmol) and proceeding successively in analogy to Procedure D (88% yield), Reference Example 2, step RE2.ii (15% yield) and Procedure B (53% yield), the title compound was obtained, after purification by CC (DCM-MeOH), as a yellowish solid (0.011 g).

¹H NMR (d6-DMSO) δ: 10.93 (s, 1H); 9.15 (s, 1H); 7.63 (s, 1H); 4.50 (t, J=5.2 Hz, 1H); 4.44 (s, 2H); 4.24-4.15 (m, 2H); 4.02 (t, J=6.5 Hz, 2H); 3.93-3.90 (m, 2H); 3.77 (m, 1H); 3.55 (m, 1H); 3.44 (m, 3H); 3.06 (s, 3H); 2.62-2.47 (overlapped m, 1H); 1.95 (m, 1H); 1.68 (quint, J=6.4 Hz, 2H); 1.53 (s, 3H).

MS (ESI, m/z): 538.1 [M+MeCN+H⁺] for C₂₃H₂₇N₃O₈S₂; t_{R}=0.65 min.

### Example 35: (2R)—N-hydroxy-4-(2-(((2R,3R)-2-(hydroxymethyl)-1-methylazetidin-3-yl)buta-1,3-diyn-1-yl)-6-oxo-4,6-dihydro-5H-thieno[2,3-c]pyrrol-5-yl)-2-methyl-2-(methylsulfonyl)butanamide

Starting from the compound of Preparation K (0.131 g; 0.3 mmol) and the compound of Preparation AH, (0.1 g; 0.48 mmol) and proceeding successively in analogy to Procedure D (68% yield) and Procedure F (53% yield), the title compound was obtained, after purification by prep-HPLC (Method 1), as a yellowish solid (0.051 g).

¹H NMR (d6-DMSO) δ: 10.92 (s, 1H); 9.15 (s, 1H); 7.60 (s, 1H); 4.70 (t, J=5.7 Hz, 1H); 4.44 (s, 2H); 3.57-3.52 (m, 2H); 3.46-3.41 (m, 3H); 3.14 (q, J=8.3 Hz, 1H); 3.05 (s, 3H); 2.99 (m, 1H); 2.71 (dd, J=6.1, 8.9 Hz, 1H); 2.60-2.47 (overlapped m, 1H); 2.24 (s, 3H); 1.95 (m, 1H); 1.53 (s, 3H).

MS (ESI, m/z): 480.06 [M+H⁺] for C₂₁H₂₅N₃O₆S₂; t_{R}=0.48 min.

### Example 36: (2R)—N-hydroxy-4-(2-(((3R,5R)-5-(hydroxymethyl)-1-methylpyrrolidin-3-yl)buta-1,3-diyn-1-yl)-6-oxo-4,6-dihydro-5H-thieno[2,3-c]pyrrol-5-yl)-2-methyl-2-(methylsulfonyl)butanamide

Starting from the compound of Preparation K (0.131 g; 0.3 mmol) and the compound of Preparation AI, (0.085 g; 0.39 mmol) and proceeding successively in analogy to Procedure D (75% yield) and Procedure F (62% yield), the title compound was obtained, after purification by prep-HPLC (Method 1), as a yellowish solid (0.067 g).

¹H NMR (d6-DMSO) δ: 10.92 (br. s, 1H); 9.17 (br. s, 1H); 7.60 (s, 1H); 4.49 (t, J=5.5 Hz, 1H); 4.43 (s, 2H); 3.54 (m, 1H); 3.46-3.37 (m, 2H); 3.27-3.19 (m, 2H); 3.13-3.05 (overlapped m, 1H); 3.05 (s, 3H); 2.57 (m, 1H); 2.40 (m, 1H); 2.28 (s, 3H); 2.26-2.22 (m, 1H); 2.02-1.89 (m, 3H); 1.53 (s, 3H).

MS (ESI, m/z): 494.07 [M+H⁺] for C₂₂H₂₇N₃O₆S₂; t_{R}=0.50 min.

### Examples 37 and 38: (2R)—N-hydroxy-2-methyl-2-(methylsulfonyl)-4-(6-oxo-2-((1-((3R)-tetrahydrofuran-3-yl)azetidin-3-yl)buta-1,3-diyn-1-yl)-4,6-dihydro-5H-thieno[2,3-c]pyrrol-5-yl)butanamide and (2R)—N-hydroxy-2-methyl-2-(methylsulfonyl)-4-(6-oxo-2-((1-((3S)-tetrahydrofuran-3-yl)azetidin-3-yl)buta-1,3-diyn-1-yl)-4,6-dihydro-5H-thieno[2,3-c]pyrrol-5-yl)butanamide

Starting from the compound of Preparation K (0.131 g; 0.3 mmol) and intermediate AJ.1 (0.083 g; 0.36 mmol), and proceeding successively in analogy to Procedure D (65% yield) and Procedure F (51% yield), one of the title compounds (hereafter “the compound of Example 37”) was obtained, after purification by prep-HPLC (Method 1), as a beige solid (0.049 g).

¹H NMR (d6-DMSO) δ: 10.92 (s, 1H); 9.15 (s, 1H); 7.61 (s, 1H); 4.44 (s, 2H); 3.69-3.61 (m, 2H); 3.58-3.38 (m, 7H); 3.06 (s, 3H); 3.07-3.00 (overlapped m, 2H); 2.97 (m, 1H); 2.57 (m, 1H); 1.94 (m, 1H); 1.75 (m, 1H); 1.57 (m, 1H); 1.53 (s, 3H).

MS (ESI, m/z): 506.1 [M+H⁺] for C₂₃H₂₇N₃O₆S₂; t_{R}=0.50 min.

Starting from the compound of Preparation K (0.12 g; 0.27 mmol) and intermediate AJ.2 (0.075 g; 0.33 mmol), and proceeding successively in analogy to Procedure D (98% yield) and Procedure F (77% yield), the other title compound (hereafter “the compound of Example 38”) was obtained, after purification by prep-HPLC (Method 1), as a beige solid (0.102 g).

¹H NMR (d6-DMSO) δ: 10.92 (s, 1H); 9.15 (s, 1H); 7.62 (s, 1H); 4.44 (s, 2H); 3.73-3.60 (m, 2H); 3.59-3.40 (m, 7H); 3.06 (s, 3H); 3.07-3.00 (overlapped m, 3H); 2.56 (m, 1H); 1.95 (m, 1H); 1.79 (m, 1H); 1.61 (m, 1H); 1.53 (s, 3H).

MS (ESI, m/z): 506.2 [M+H⁺] for C₂₃H₂₇N₃O₆S₂; t_{R}=0.50 min.

The absolute stereochemistry of the C-3 of the tetrahydofuran ring of the compounds of Examples 37 and 38 has not been assigned.

### Example 39: (2R)—N-hydroxy-4-(2-((1-((3-hydroxyoxetan-3-yl)methyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide

Starting from the compound of Preparation K (0.086 g; 0.19 mmol) and the compound of Preparation AK (0.057 g; 0.23 mmol), and proceeding successively in analogy to Procedure D (72% yield) and Procedure B (23% yield), the title compound was obtained, after purification by prep-HPLC (Method 1), as a beige solid (0.016 g).

¹H NMR (d6-DMSO) δ: 10.88 (m, 1H); 9.11 (m, 1H); 7.61 (s, 1H); 5.55 (s, 1H); 4.44 (s, 2H); 4.32-4.34 (m, 2H); 4.28-4.30 (m, 2H); 3.29-3.61 (m, 5H); 3.14-3.19 (m, 2H); 3.06 (s, 3H); 2.63-2.67 (m, 2H); 2.57 (m, 1H); 1.95 (m, 1H): 1.53 (s, 3H).

MS (ESI, m/z): 522.3 [M+H⁺] for C₂₃H₂₇N₃O₇S₂; t_{R}=0.50 min.

### Example 40: (2R)—N-hydroxy-4-(2-(((2R,4R)-4-(hydroxymethyl)-1-methylazetidin-2-yl)buta-1,3-diyn-1-yl)-6-oxo-4,6-dihydro-5H-thieno[2,3-c]pyrrol-5-yl)-2-methyl-2-(methylsulfonyl)butanamide

Starting from the compound of Preparation K (0.078 g; 0.18 mmol) and the compound of Preparation AL (0.043 g; 0.21 mmol), and proceeding successively in analogy to Procedure D (57% yield) and Procedure B (61% yield), the title compound was obtained, after purification by prep-HPLC (Method 1), as a beige solid (0.028 g).

¹H NMR (d6-DMSO) δ: 10.92 (br. s, 1H); 9.16 (s, 1H); 7.66 (s, 1H); 4.57 (t, J=5.4 Hz, 1H); 4.50-4.42 (m, 2H); 4.31 (m, 1H); 3.56 (m, 1H); 3.45 (m, 1H); 3.42-3.39 (m, 2H); 3.28 (m, 1H); 3.07 (s, 3H); 2.58 (ddd, J=6.6, 9.6, 13.1 Hz, 1H); 2.28 (s, 3H); 2.24 (overlapped m, 1H); 2.00 (m, 1H); 1.95 (m, 1H); 1.54 (s, 3H).

MS (ESI, m/z): 480.1.3 [M+H⁺] for C₂₁H₂₅N₃O₆S₂; t_{R}=0.50 min.

### Example 41: (2R)-4-(2-(((2S,3S)-2-(fluoromethyl)-1-methylazetidin-3-yl)buta-1,3-diyn-1-yl)-6-oxo-4,6-dihydro-5H-thieno[2,3-c]pyrrol-5-yl)-N-hydroxy-2-methyl-2-(methylsulfonyl)butanamide

Starting from the compound of Preparation K (0.078 g; 0.18 mmol) and the compound of Preparation AM (0.073 g; 0.35 mmol), and proceeding successively in analogy to Procedure D (34% yield) and Procedure B (66% yield), the title compound was obtained, after purification by prep-HPLC (Method 1), as a beige solid (0.035 g).

¹H NMR (d6-DMSO) δ: 10.93 (br. s, 1H); 9.16 (s, 1H); 7.63 (s, 1H); 4.53-4.35 (m, 4H); 3.60 (dd, J=6.0, 7.2 Hz, 1H); 3.55 (m, 1H); 3.44 (m, 1H); 3.29 (overlapped m, 1H); 3.22 (m, 1H); 3.06 (s, 3H); 2.80 (m, 1H); 2.59 (m, 1H); 2.27 (s, 3H); 1.95 (m, 1H); 1.53 (s, 3H).

MS (ESI, m/z): 482.1 [M+H⁺] for C₂₁H₂₄N₃O₅FS₂; t_{R}=0.51 min.

### Example 42: (2R)—N-hydroxy-2-methyl-2-(methylsulfonyl)-4-(6-oxo-2-(phenylethynyl)-4,6-dihydro-5H-thieno[2,3-c]pyrrol-5-yl)butanamide

Starting from the compound of Preparation K (0.1 g; 0.22 mmol) and iodobenzene (0.055 g; 0.27 mmol), and proceeding successively in analogy to Procedure A (64% yield) and Procedure B (34% yield), the title compound was obtained, after purification by CC (DCM-MeOH), as a yellowish solid (0.049 g).

¹H NMR (d6-DMSO) δ: 10.95 (s, 1H); 9.17 (s, 1H); 7.63-7.59 (m, 2H); 7.56 (s, 1H); 7.49-7.45 (m, 3H); 4.47 (s, 2H); 3.57 (m, 1H); 3.45 (m, 1H); 3.08 (s, 3H); 2.59 (m, 1H); 1.97 (m, 1H); 1.55 (s, 3H).

MS (ESI, m/z): 433.10 [M+H⁺] for C₂₀H₂₀N₂O₅S₂; t_{R}=0.75 min.

The racemic mixtures of Reference Examples 1 to 2 can be separated into their enantiomers using, for example, chiral HPLC. Thus the following further invention compounds or salts thereof can be obtained:
(2R)—N-hydroxy-4-(2-((4-(1-hydroxy-2-methylpropan-2-yl)phenyl)ethynyl)-6-oxo-4,6-dihydro-5H-thieno[2,3-c]pyrrol-5-yl)-2-methyl-2-(methylsulfonyl)butanamide; and
(2R)—N-hydroxy-4-(2-((1-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)butanamide.

Pharmacological Properties of the Invention Compounds

In Vitro Assays

Bacterial Growth Minimal Inhibitory Concentrations:

Experimental Methods:

Minimal Inhibitory Concentrations (MICs; mg/L) were determined in cation-adjusted Mueller-Hinton Broth by a microdilution method following the description given in “Methods for Dilution Antimicrobial Susceptibility Tests for Bacteria that grow Aerobically”, Approved standard, 7^{th} ed., Clinical and Laboratory Standards Institute (CLSI) Document M7-A7, Wayne, Pa., USA (2006).

Results:

All Example compounds were tested against several Gram-positive and Gram-negative bacteria. Typical antibacterial test results are given in Table 1 hereafter (MICs in mg/L). K. pneumoniae A-651 is a multiply-resistant strain (in particular quinolone-resistant), while E. coli ATCC25922 and P. aeruginosa ATCC27853 are quinolone-sensitive strains.

TABLE 1

| | MIC for | MIC for | MIC for |
|---|---|---|---|
| Example | E. coli | P. aeruginosa | K. Pneumoniae |
| No. | ATCC25922 | ATCC27853 | A-651 |
| RE1 | 0.25 | 2 | 1 |
| RE2 | 1 | 2 | 2 |
| 1 | 0.125 | 0.5 | 0.25 |
| 2 | ≤0.063 | 2 | 0.25 |
| 3 | 0.25 | 1 | 0.5 |
| 4 | 0.25 | 0.5 | 0.5 |
| 5 | 0.125 | 1 | 0.25 |
| 6 | 1 | 2 | 1 |
| 7 | 0.25 | 2 | 1 |
| 8 | 1 | 1 | 2 |
| 9 | 1 | 2 | 1 |
| 10 | 0.125 | 8 | 0.063 |
| 11 | 0.25 | 0.5 | 1 |
| 12 | 0.031 | 2 | 0.25 |
| 13 | 0.5 | 1 | 1 |
| 14 | 0.031 | 2 | 0.25 |
| 15 | 0.5 | 4 | 4 |
| 17 | 0.5 | 1 | 2 |
| 18 | 0.5 | 1 | 2 |
| 19 | 0.125 | 0.5 | 0.25 |
| 20 | 0.125 | 1 | 0.25 |
| 21 | 0.5 | 1 | 2 |
| 22 | 0.5 | 1 | 1 |
| 23 | 0.063 | 1 | 0.25 |
| 24 | 0.25 | 1 | 0.5 |
| 25 | 1 | 1 | 1 |
| 26 | 0.25 | 2 | 0.25 |
| 27 | 0.125 | 1 | 0.25 |
| 28 | 0.25 | 2 | 0.5 |
| 29 | 0.25 | 2 | 1 |
| 30 | 0.125 | 2 | 1 |
| 31 | 0.5 | 1 | 0.5 |
| 32 | 0.25 | 1 | 0.5 |
| 33 | 0.125 | 2 | 0.5 |
| 34 | 0.25 | 1 | 0.5 |
| 35 | 0.5 | 1 | 2 |
| 36 | 0.5 | 1 | 1 |
| 37 | 0.5 | 1 | 0.5 |
| 38 | 0.25 | 1 | 0.25 |
| 39 | 1 | 2 | 2 |
| 40 | 0.25 | 1 | 2 |
| 41 | 0.125 | 1 | 0.25 |
| 42 | ≤0.031 | 2 | 0.125 |
| Cipro | ≤0.063 | 0.125 | >8 |

The compounds of Examples 4 and 16 were tested against wild-type E. coli A-1261 in the absence of alkaline phosphatase or esterase, in the presence of an alkaline phosphatase, and in the presence of an esterase. The corresponding antibacterial test results are given in Table 2 hereafter (MICs in mg/L).

TABLE 2

| | MIC for E. coli A-1261 | | |
|---|---|---|---|
| | In the absence | In the presence | |
| | of alkaline | of an alkaline | In the presence |
| Example | phosphatase | phosphatase | of an esterase |
| No. | or esterase | (2 i.U./mL) | (10 i.U./mL) |
| 4 | 0.125 | 0.25 | 0.25 |
| 16 | >8 | 0.25 | >8 |

1. A compound of formula I 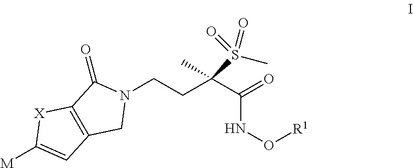
wherein
X represents sulphur or CH═CH;
R¹ represents H, PO₃H₂, SO₃H, phosphonooxymethyl or the group L represented below 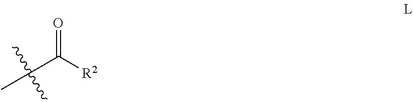
wherein
R² represents (C₁-C₄)alkylamino(C₁-C₄)alkyl, di(C₁-C₄)alkylamino(C₁-C₄)alkyl, phosphonooxy(C₁-C₄)alkyl, phosphonooxymethoxy, 2-(phosphonooxy-(C₁-C₄)alkyl)-phenyl, [2-(phosphonooxy-(C₁-C₄)alkyl)-phenyl]-(C₁-C₄)alkyl, or (2-(phosphonooxy)-phenyl)-(C₁-C₄)alkyl;
M is one of the groups M^{A} and M^{B} represented below 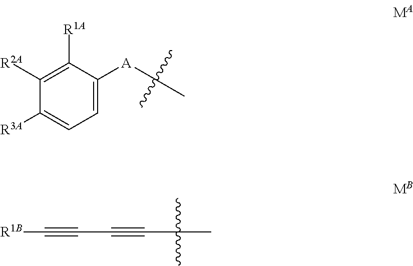
wherein A represents a bond or C≡C;
R^{1A} represents H or halogen;
R^{2A} represents H or halogen;
R^{3A} represents H, (C₁-C₃)alkoxy, hydroxy(C₂-C₄)alkoxy, hydroxy(C₁-C₄)alkyl, 1,2-dihydroxyethyl, (3-fluoroazetidin-1-yl)methyl, 3-fluoro-1-(oxetan-3-yl)azetidin-3-yl, 3-fluoro-1-methyl-azetidin-3-yl, (4-hydroxy-3-fluoropiperidin-1-yl)methyl, (4-hydroxy-3,3-difluoropiperidin-1-yl)methyl, (3-hydroxyazetidin-1-yl)methyl, 3-(ω-hydroxy(C₂-C₄)alkyl)-azetidin-1-yl, 1-(oxetan-3-yl)azetidin-3-yl, 1-(oxetan-3-ylmethyl)azetidin-3-yl or (4-hydroxypiperidin-1-yl)methyl; and
R^{1B} represents amino(C₁-C₃)alkyl, 1-amino-cycloprop-1-yl, trans-2-(2-dimethylaminoacetoxymethyl)-cycloprop-1-yl, 1-(2-dimethylaminoacetoxymethyl)-cycloprop-1-yl, 1-(3-hydroxyazetidine)-1-carbonyloxymethyl, 1-hydroxymethyl-cycloprop-1-yl, trans-2-hydroxymethyl-cycloprop-1-yl, 1-fluoro-2-hydroxymethyl-cycloprop-1-yl, 2-fluoro-2-hydroxymethyl-cycloprop-1-yl, 1-{[(2-(phosphonooxy-(C₁-C₄)alkyl)-phenyl)-(C₁-C₄)alkyl]carbonyloxymethyl}cycloprop-1-yl, 1-{[2-(phosphonooxy-(C₁-C₄)alkyl)-phenyl]carbonyloxymethyl}cycloprop-1-yl, 1-{[(2-phosphonooxy-phenyl)-(C₁-C₄)alkyl]carbonyloxymethyl}cycloprop-1-yl, trans-2-{[(2-(phosphonooxy-(C₁-C₄)alkyl)-phenyl)-(C₁-C₄)alkyl]carbonyloxymethyl}cycloprop-1-yl, trans-2-{[2-(phosphonooxy-(C₁-C₄)alkyl)-phenyl]carbonyloxymethyl}cycloprop-1-yl, trans-2-{[(2-phosphonooxy-phenyl)-(C₁-C₄)alkyl]carbonyloxymethyl}cycloprop-1-yl, 2-fluoro-2-{[(2-(phosphonooxy-(C₁-C₄)alkyl)-phenyl)-(C₁-C₄)alkyl]carbonyloxymethyl}cycloprop-1-yl, 2-fluoro-2-{[2-(phosphonooxy-(C₁-C₄)alkyl)-phenyl]carbonyloxymethyl}cycloprop-1-yl, 2-fluoro-2-{[(2-phosphonooxy-phenyl)-(C₁-C₄)alkyl]carbonyloxymethyl}cycloprop-1-yl, 2-fluoro-2-(phosphonooxymethyl)-cycloprop-1-yl, 1-methyl-2-hydroxymethyl-cycloprop-1-yl, 2-hydroxymethyl-2-methylcycloprop-1-yl, 1-(2-hydroxyacetyl)-azetidin-3-yl, trans-(cis-3,4-dihydroxy)-cyclopent-1-yl, 2-(1,2-dihydroxyethyl)cycloprop-1-yl, 1-(dimethylamino)cycloprop-1-yl, 2-(morpholinomethyl)cycloprop-1-yl, 2-((3-fluoroazetidin-1-yl)methyl)cyclopropyl, N—(C₁-C₄)alkyl-azetidin-3-yl, N—(C₃-C₆)cycloalkyl-azetidin-3-yl, 3-fluoro-1-methyl-azetidin-3-yl, 1-(methylamino)cycloprop-1-yl, N-(ω-hydroxy(C₂-C₄)alkyl)-azetidin-3-yl, 2-(hydroxymethyl)-1-methylazetidin-3-yl, N-(2-hydroxy-2-methylpropyl)azetidin-3-yl, N-(ω-halo(C₂-C₄)alkyl)-azetidin-3-yl, 1-(3-hydroxypropyloxycarbonyl)-azetidin-3-yl, 2-(fluoromethyl)-1-methylazetidin-3-yl, N-(3-hydroxycyclobutyl)azetidin-3-yl, N-(oxetan-3-ylmethyl)azetidin-3-yl, N-(3-hydroxyoxetan-3-ylmethyl)azetidin-3-yl, N-(tetrahydrofuran-3-yl)azetidin-3-yl, N-(tetrahydro-2H-pyran-4-yl)azetidin-3-yl, trans-2-hydroxymethyl-1-methylazetidin-4-yl, (5-hydroxymethyl)-1-methylpyrrolidin-3-yl, 4-fluoro-1-methylpiperidin-4-yl, or 1-(oxetan-3-yl)-azetidin-3-yl;
or a salt thereof.

2. A compound of formula I according to claim 1, which is also a compound of formula I_{P} 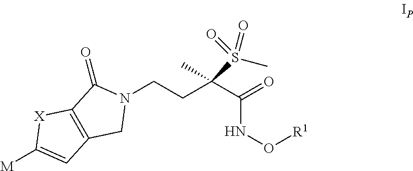
wherein
X represents sulphur or CH═CH;
R¹ represents H, PO₃H₂, SO₃H, phosphonooxymethyl or the group L represented below 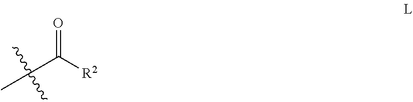
wherein
R² represents (C₁-C₄)alkylamino(C₁-C₄)alkyl, di(C₁-C₄)alkylamino(C₁-C₄)alkyl, phosphonooxy(C₁-C₄)alkyl, phosphonooxymethoxy, 2-(phosphonooxy-(C₁-C₄)alkyl)-phenyl, [2-(phosphonooxy-(C₁-C₄)alkyl)-phenyl]-(C₁-C₄)alkyl, or (2-(phosphonooxy)-phenyl)-(C₁-C₄)alkyl;
M is one of the groups M^{A} and M^{B} represented below 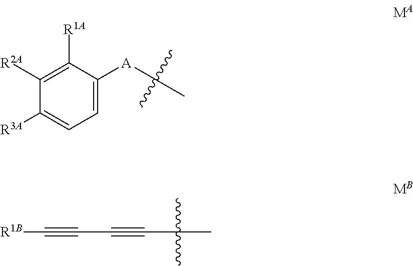
wherein A represents a bond or C≡C;
R^{1A} represents H or halogen;
R^{2A} represents H or halogen;
R^{3A} represents (C₁-C₃)alkoxy, hydroxy(C₂-C₄)alkoxy, hydroxy(C₁-C₄)alkyl, 1,2-dihydroxyethyl, (3-fluoroazetidin-1-yl)methyl, 3-fluoro-1-(oxetan-3-yl)azetidin-3-yl, 3-fluoro-1-methyl-azetidin-3-yl, (4-hydroxy-3-fluoropiperidin-1-yl)methyl, (4-hydroxy-3,3-difluoropiperidin-1-yl)methyl, (3-hydroxyazetidin-1-yl)methyl, 3-(ω-hydroxy(C₂-C₄)alkyl)-azetidin-1-yl, or (4-hydroxypiperidin-1-yl)methyl; and
R^{1B} represents amino(C₁-C₃)alkyl, 1-amino-cycloprop-1-yl, trans-2-(2-dimethylaminoacetoxymethyl)-cycloprop-1-yl, 1-(2-dimethylaminoacetoxymethyl)-cycloprop-1-yl, 1-(3-hydroxyazetidine)-1-carbonyloxymethyl, 1-hydroxymethyl-cycloprop-1-yl, trans-2-hydroxymethyl-cycloprop-1-yl, 1-fluoro-2-hydroxymethyl-cycloprop-1-yl, 2-fluoro-2-hydroxymethyl-cycloprop-1-yl, 1-{[(2-(phosphonooxy-(C₁-C₄)alkyl)-phenyl)-(C₁-C₄)alkyl]carbonyloxymethyl}cycloprop-1-yl, 1-{[2-(phosphonooxy-(C₁-C₄)alkyl)-phenyl]carbonyloxymethyl}cycloprop-1-yl, 1-{[(2-phosphonooxy-phenyl)-(C₁-C₄)alkyl]carbonyloxymethyl}cycloprop-1-yl, trans-2-{[(2-(phosphonooxy-(C₁-C₄)alkyl)-phenyl)-(C₁-C₄)alkyl]carbonyloxymethyl}cycloprop-1-yl, trans-2-{[2-(phosphonooxy-(C₁-C₄)alkyl)-phenyl]carbonyloxymethyl}cycloprop-1-yl, trans-2-{[(2-phosphonooxy-phenyl)-(C₁-C₄)alkyl]carbonyloxymethyl}cycloprop-1-yl, 2-fluoro-2-{[(2-(phosphonooxy-(C₁-C₄)alkyl)-phenyl)-(C₁-C₄)alkyl]carbonyloxymethyl}cycloprop-1-yl, 2-fluoro-2-{[2-(phosphonooxy-(C₁-C₄)alkyl)-phenyl]carbonyloxymethyl}cycloprop-1-yl, 2-fluoro-2-{[(2-phosphonooxy-phenyl)-(C₁-C₄)alkyl]carbonyloxymethyl}cycloprop-1-yl, 2-fluoro-2-(phosphonooxymethyl)-cycloprop-1-yl, 1-methyl-2-hydroxymethyl-cycloprop-1-yl, 2-hydroxymethyl-2-methylcycloprop-1-yl, 1-(2-hydroxyacetyl)-azetidin-3-yl, trans-(cis-3,4-dihydroxy)-cyclopent-1-yl, 2-(1,2-dihydroxyethyl)cycloprop-1-yl, 1-(dimethylamino)cycloprop-1-yl, 2-(morpholinomethyl)cycloprop-1-yl, N—(C₁-C₄)alkyl-azetidin-3-yl, N—(C₃-C₆)cycloalkyl-azetidin-3-yl, 3-fluoro-1-methyl-azetidin-3-yl, 1-(methylamino)cycloprop-1-yl, N-(ω-hydroxy(C₂-C₄)alkyl)-azetidin-3-yl, 4-fluoro-1-methylpiperidin-4-yl, or 1-(oxetan-3-yl)-azetidin-3-yl;
or a salt thereof.

3. A compound according to claim 1, wherein X represents sulphur, or a salt thereof.

4. A compound according to claim 1, wherein X represents CH═CH, or a salt thereof.

5. A compound according to claim 1, wherein R¹ represents H, or a salt thereof.

6. A compound according to claim 1, wherein M represents the group M^{A}, or a salt thereof.

7. A compound according to claim 6, wherein A represents a bond, or a salt thereof.

8. A compound according to claim 6, wherein A represents C≡C, or a salt thereof.

9. A compound according to claim 6, wherein R^{1A} represents H, or a salt thereof.

10. A compound according to claim 6, wherein R^{1A} represents halogen, or a salt thereof.

11. A compound according to claim 10, wherein R^{1A} represents fluorine, or a salt thereof.

12. A compound according to claim 6, wherein R^{2A} represents H, or a salt thereof.

13. A compound according to claim 4, wherein R^{3A} represents (C₁-C₃)alkoxy or (3-fluoroazetidin-1-yl)methyl, or a salt thereof.

14. A compound according to claim 4, wherein R^{3A} represents hydroxy(C₁-C₄)alkyl, or a salt thereof.

15. A compound according to claim 1, wherein M represents the group M^{B}, or a salt thereof.

16. A compound according to claim 15, wherein R^{1B} represents 1-amino-cycloprop-1-yl, trans-2-(2-dimethylaminoacetoxymethyl)-cycloprop-1-yl, cis-2-fluoro-2-hydroxymethyl-cycloprop-1-yl, cis-2-fluoro-2-(phosphonooxymethyl)-cycloprop-1-yl, 1-hydroxymethyl-cycloprop-1-yl, trans-2-hydroxymethyl-cycloprop-1-yl, or N—(C₁-C₄)alkyl-azetidin-3-yl, or a salt thereof.

17. A compound according to claim 15, wherein R^{1B} represents trans-(cis-3,4-dihydroxy)-cyclopent-1-yl, 1-(3-hydroxyazetidine)-1-carbonyloxymethyl, N-(ω-hydroxy(C₂-C₄)alkyl)-azetidin-3-yl, or 1-(methylamino)cycloprop-1-yl, or a salt thereof.

18. A compound according to claim 1, wherein
X represents sulphur or CH═CH;
R¹ represents H;
M represents M^{A} or M^{B},
wherein A represents a bond or C≡C,
R^{1A} represents H or halogen,
R^{2A} represents H, and
R^{3A} represents (C₁-C₃)alkoxy, (3-fluoroazetidin-1-yl)methyl or hydroxy(C₁-C₄)alkyl;
and wherein R^{1B} represents 1-amino-cycloprop-1-yl, trans-(cis-3,4-dihydroxy)-cyclopent-1-yl, trans-2-(2-dimethylaminoacetoxymethyl)-cycloprop-1-yl, cis-2-fluoro-2-hydroxymethyl-cycloprop-1-yl, cis-2-fluoro-2-(phosphonooxymethyl)-cycloprop-1-yl, 1-(3-hydroxyazetidine)-1-carbonyloxymethyl, N-(ω-hydroxy(C₂-C₄)alkyl)-azetidin-3-yl, 1-hydroxymethyl-cycloprop-1-yl, trans-2-hydroxymethyl-cycloprop-1-yl, 1-(methylamino)cycloprop-1-yl, N—(C₁-C₄)alkyl-azetidin-3-yl, or 1-(oxetan-3-yl)-azetidin-3-yl;
or a salt thereof.

19. A compound according to claim 1, which is selected from the group consisting of:
(2R)—N-hydroxy-4-(2-((4-(1-hydroxy-2-methylpropan-2-yl)phenyl)ethynyl)-6-oxo-4,6-dihydro-5H-thieno[2,3-c]pyrrol-5-yl)-2-methyl-2-(methylsulfonyl)butanamide;
(2R)—N-hydroxy-4-(2-((1-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-4-(2-(((1R,2R)-2-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)butanamide;
(2R)-4-(2-(2-fluoro-4-methoxyphenyl)-6-oxo-4,6-dihydro-5H-thieno[2,3-c]pyrrol-5-yl)-N-hydroxy-2-methyl-2-(methylsulfonyl)butanamide;
(2R)-4-(2-((1-aminocyclopropyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide;
(2R)-4-(2-(((1R,2R)-2-fluoro-2-(hydroxymethyl)cyclopropyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide;
(2R)—N-hydroxy-2-methyl-4-(2-((1-(methylamino)cyclopropyl)buta-1,3-diyn-1-yl)-6-oxo-4,6-dihydro-5H-thieno[2,3-c]pyrrol-5-yl)-2-(methylsulfonyl)butanamide;
(3R)-5-(5-(4-(hydroxyamino)-3-methyl-3-(methylsulfonyl)-4-oxobutyl)-6-oxo-5,6-dihydro-4H-thieno[2,3-c]pyrrol-2-yl)penta-2,4-diyn-1-yl 3-hydroxyazetidine-1-carboxylate;
(2R)—N-hydroxy-2-methyl-4-(5-((1-(methylamino)cyclopropyl)buta-1,3-diyn-1-yl)-1-oxoisoindolin-2-yl)-2-(methylsulfonyl)butanamide;
(2R)—N-hydroxy-2-methyl-4-(5-((1-methylazetidin-3-yl)buta-1,3-diyn-1-yl)-1-oxoisoindolin-2-yl)-2-(methylsulfonyl)butanamide;
(3R)-5-(2-(4-(hydroxyamino)-3-methyl-3-(methylsulfonyl)-4-oxobutyl)-1-oxoisoindolin-5-yl)penta-2,4-diyn-1-yl 3-hydroxyazetidine-1-carboxylate;
(2R)-4-(5-(2-fluoro-4-methoxyphenyl)-1-oxoisoindolin-2-yl)-N-hydroxy-2-methyl-2-(methylsulfonyl)butanamide;
(2R)—N-hydroxy-2-methyl-4-(2-((1-methylazetidin-3-yl)buta-1,3-diyn-1-yl)-6-oxo-4,6-dihydro-5H-thieno[2,3-c]pyrrol-5-yl)-2-(methylsulfonyl)butanamide;
(2R)—N-hydroxy-4-(5-((1-(hydroxymethyl)cyclopropyl)buta-1,3-diyn-1-yl)-1-oxoisoindolin-2-yl)-2-methyl-2-(methylsulfonyl)butanamide;
(2R)-4-(5-((1-aminocyclopropyl)buta-1,3-diyn-1-yl)-1-oxoisoindolin-2-yl)-N-hydroxy-2-methyl-2-(methylsulfonyl)butanamide;
(2R)-4-(2-((4-((3-fluoroazetidin-1-yl)methyl)phenyl)ethynyl)-6-oxo-4,6-dihydro-5H-thieno[2,3-c]pyrrol-5-yl)-N-hydroxy-2-methyl-2-(methylsulfonyl)butanamide;
(2R)—N-hydroxy-2-methyl-2-(methylsulfonyl)-4-(2-((1-(oxetan-3-yl)azetidin-3-yl)buta-1,3-diyn-1-yl)-6-oxo-4,6-dihydro-5H-thieno[2,3-c]pyrrol-5-yl)butanamide;
((1R,2R)-1-fluoro-2-((5-((3R)-4-(hydroxyamino)-3-methyl-3-(methylsulfonyl)-4-oxobutyl)-6-oxo-5,6-dihydro-4H-thieno[2,3-c]pyrrol-2-yl)buta-1,3-diyn-1-yl)cyclopropyl)methyl dihydrogen phosphate;
(2R)—N-hydroxy-4-(2-((1-(2-hydroxyethyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)-4-(5-(((1R,2R)-2-fluoro-2-(hydroxymethyl)cyclopropyl)buta-1,3-diyn-1-yl)-1-oxoisoindolin-2-yl)-N-hydroxy-2-methyl-2-(methylsulfonyl)butanamide;
(2R)—N-hydroxy-4-(2-(((1S,2S)-2-(hydroxymethyl)cyclopropyl)buta-1,3-diyn-1-yl)-6-oxo-4,6-dihydro-5H-thieno[2,3-c]pyrrol-5-yl)-2-methyl-2-(methylsulfonyl)butanamide;
((1S,2S)-2-((5-((3R)-4-(hydroxyamino)-3-methyl-3-(m ethylsulfonyl)-4-oxobutyl)-6-oxo-5,6-dihydro-4H-thieno[2,3-c]pyrrol-2-yl)buta-1,3-diyn-1-yl)cyclopropyl)methyl dimethylglycinate; and
(2R)-4-(2-(((1S,3R,4S)-3,4-dihydroxycyclopentyl)buta-1,3-diyn-1-yl)-6-oxo-4,6-dihydro-5H-th ieno[2,3-c]pyrrol-5-yl)-N-hydroxy-2-methyl-2-(methylsulfonyl)butanamide;
or a salt of such compound.

20. A compound according to claim 1, which is selected from the group consisting of:
(2R)-4-(2-(((1S,2S)-2-((2R)-1,2-dihydroxyethyl)cyclopropyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide;
(2R)-4-(2-((1-(2-fluoroethyl)azetidin-3-yl)buta-1,3-diyn-1-yl)-6-oxo-4,6-dihydro-5H-thieno[2,3-c]pyrrol-5-yl)-N-hydroxy-2-methyl-2-(methylsulfonyl)butanamide;
(R)—N-hydroxy-4-(2-((1-isopropylazetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-4-(2-((1-(2-hydroxy-2-methylpropyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-2-methyl-2-(methylsulfonyl)-4-(2-((4-(1-(oxetan-3-yl)azetidin-3-yl)phenyl)ethynyl)-6-oxo-4,6-dihydro-5H-thieno[2,3-c]pyrrol-5-yl)butanamide;
(2R)—N-hydroxy-2-methyl-2-(methylsulfonyl)-4-(6-oxo-2-((1-(tetrahydro-2H-pyran-4-yl)azetidin-3-yl)buta-1,3-diyn-1-yl)-4,6-dihydro-5H-thieno[2,3-c]pyrrol-5-yl)butanamide;
(2R)—N-hydroxy-2-methyl-2-(methylsulfonyl)-4-(2-((1-(oxetan-3-ylmethyl)azetidin-3-yl)buta-1,3-diyn-1-yl)-6-oxo-4,6-dihydro-5H-thieno[2,3-c]pyrrol-5-yl)butanamide;
(2R)—N-hydroxy-2-methyl-2-(methylsulfonyl)-4-(2-((4-(1-(oxetan-3-yl)azetidin-3-yl)phenyl)ethynyl)-6-oxo-4,6-dihydro-5H-thieno[2,3-c]pyrrol-5-yl)butanamide;
(2R)-4-(2-(((1S,2S)-2-((3-fluoroazetidin-1-yl)methyl)cyclopropyl)buta-1,3-diyn-1-yl)-6-oxo-4,6-dihydro-5H-thieno[2,3-c]pyrrol-5-yl)-N-hydroxy-2-methyl-2-(methylsulfonyl)butanamide;
(2R)—N-hydroxy-4-(2-((1-((1s,3s)-3-hydroxycyclobutyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-4-(2-((1-((1r,3r)-3-hydroxycyclobutyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-4-(5-((4-(2-hydroxyethyl)phenyl)ethynyl)-1-oxoisoindolin-2-yl)-2-methyl-2-(methylsulfonyl)butanamide;
3-hydroxypropyl (2R)-3-((5-(4-(hydroxyamino)-3-methyl-3-(methylsulfonyl)-4-oxobutyl)-6-oxo-5,6-dihydro-4H-thieno[2,3-c]pyrrol-2-yl)buta-1,3-diyn-1-yl)azetidine-1-carboxylate;
(2R)—N-hydroxy-4-(2-(((2R,3R)-2-(hydroxymethyl)-1-methylazetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-4-(2-(((3R,5R)-5-(hydroxymethyl)-1-methylpyrrolidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-2-methyl-2-(methylsulfonyl)-4-(6-oxo-2-((1-((3R)-tetrahydrofuran-3-yl)azetidin-3-yl)buta-1,3-diyn-1-yl)-4,6-dihydro-5H-thieno[2,3-c]pyrrol-5-yl)butanamide;
(2R)—N-hydroxy-2-methyl-2-(methylsulfonyl)-4-(6-oxo-2-((1-((3S)-tetrahydrofuran-3-yl)azetidin-3-yl)buta-1,3-diyn-1-yl)-4,6-dihydro-5H-thieno[2,3-c]pyrrol-5-yl)butanamide;
(2R)—N-hydroxy-4-(2-((1-((3-hydroxyoxetan-3-yl)methyl)azetidin-3-yl)buta-1,3-diyn-1-yl)-6-oxo-4,6-dihydro-5H-thieno[2,3-c]pyrrol-5-yl)-2-methyl-2-(methylsulfonyl)butanamide;
(2R)—N-hydroxy-4-(2-(((2R,4R)-4-(hydroxymethyl)-1-methylazetidin-2-yl)buta-1,3-diyn-1-yl)-6-oxo-4,6-dihydro-5H-thieno[2,3-c]pyrrol-5-yl)-2-methyl-2-(methylsulfonyl)butanamide;
(2R)-4-(2-(((2S,3S)-2-(fluoromethyl)-1-methylazetidin-3-yl)buta-1,3-diyn-1-yl)-6-oxo-4,6-dihydro-5H-thieno[2,3-c]pyrrol-5-yl)-N-hydroxy-2-methyl-2-(methylsulfonyl)butanamide; and
(2R)—N-hydroxy-2-methyl-2-(methylsulfonyl)-4-(6-oxo-2-(phenylethynyl)-4,6-dihydro-5H-thieno[2,3-c]pyrrol-5-yl)butanamide;
or a salt of such compound.

21. As a medicament, a compound according to claim 1, or a pharmaceutically acceptable salt thereof.

22. A pharmaceutical composition containing, as active ingredient, a compound according to claim 1, or a pharmaceutically acceptable salt thereof, and at least one therapeutically inert excipient.

23. A compound according to claim 1, or a pharmaceutically acceptable salt thereof, for the prevention or treatment of a bacterial infection.

24. A compound or pharmaceutically acceptable salt according to claim 23, which is for the prevention or treatment of a Gram-negative bacterial infection.
